(19) United States
(12) Patent Application Publication
(10) Pub. No.: US 2023/0118435 A1
(43) Pub. Date: Apr. 20, 2023

(54) COMPOSITION COMPRISING AN AVENANTHRAMIDE WITH IMPROVED SKIN PENETRATION

(71) Applicant: SYMRISE AG, Holzminden (DE)

(72) Inventors: Sabine Lange, Holzminden (DE); Martina Herrmann, Hameln (DE); Imke Meyer, Bodenwerder (DE); Sebastian Bruncke, Höxter (DE); Holger Joppe, Dassel (DE); Kalpana Palani, Holzminden (DE)

(21) Appl. No.: 17/909,340

(22) PCT Filed: Mar. 5, 2021
(86) PCT No.: PCT/EP2021/055627

§ 371 Date: Sep. 2, 2022

(30) Foreign Application Priority Data
Mar. 6, 2020 (EP) PCT/EP2020/056120

(51) Int. Cl. A61K 31/196; A61K 8/44; A61Q 19/10; A61Q 5/02

(52) U.S. Cl. CPC A61K 31/196; A61K 8/445; A61Q 19/10; A61Q 5/02

(57) ABSTRACT

The present invention relates generally to: a composition comprising or consisting of a specific avenanthramide and at least one penetration enhancer with an improved skin penetration; a method for preparing said composition; the use of such compositions as cosmetics or pharmaceuticals; and cosmetics or pharmaceuticals comprising said composition.

### TECHNICAL FIELD

The present invention relates generally to: a composition comprising or consisting of a specific avenanthramide and at least one penetration enhancer with an improved skin penetration; a method for preparing said composition; the use of such compositions as cosmetics or pharmaceuticals; and cosmetics or pharmaceuticals comprising said composition.

### BACKGROUND ART

Avenanthramides (in the following abbreviated as Avns or Avn for a single avenanthramide compound), which are low-molecular-weight phenolic amides containing anthranilic acid and hydroxycinnamic acid moieties with an amide bond, are a group of naturally occurring phenolic amides in oats, both A. sativa and A. nuda. They were originally identified as phytoalexins produced by the plant in response to exposure to pathogens, such as fungi. Oats contain a unique group of approximately 40 different types of Avns, which are present in both oat grains and leaves. The most abundant are Avn A (N-(4′-hydroxycinnamoyl)-5-hydroxyanthranilic acid), Avn B (N-(4′-hydroxy-3′-methoxycinnamoyl)-5-hydroxyanthranilic acid) and Avn C (N-(3′-4′-dihydroxycinnamoyl)-5-hydroxyanthranilic acid), which are amides of 5-hydroxyanthranilic acid with p-coumaric, ferulic and caffeic hydroxycinnamic acids, respectively. These Avns are constitutively expressed in the kernels, appearing in almost all milling fractions, but occur at their highest concentrations in the bran and outer layers of the kernel [Boz H, Czech Journal of Food Sciences 2015, 33(5): 399-404]. The total content of avenanthramides (Avns) in oat grain has been found to be about 2 to 700 mg/kg (0.0002 to 0.07%), depending on the cultivar and agronomic treatment [Maliarova M et al., Journal of the Brazilian Chemical Society 2015, 26(11), 2369-2378].

The extraction of Avns from oats is carried out using various solvent compositions such as pure or diluted ethanol and methanol. Extraction procedures were achieved over different times at room temperature or under controlled heating, such as naked oats, 50% aqueous ethanol [Tong L. et al., Journal of Integrative Agriculture 2014, 13, 1809].

Maliarova, M. et al., Journal of the Brazilian Chemical Society 2015, 26(11), 2369-2378 compared the efficiency of methanol, ethanol and isopropanol on the extraction of Avns from naked oat bran. The optimum conditions for the highest yield of Avns were a methanol concentration of 70%, an extraction temperature of 55° C. and an extraction time of 165 minutes.

The application of avenanthramides is a growing field in cosmetics and/or therapeutics since a number of studies have demonstrated that avenanthramides have excellent antioxidant activity both in vitro and in vivo, as well as anti-inflammatory, anti-irritant, anti-atherogenic and anti-proliferative activities which may prevent or limit cellular oxidative dysfunctions and the development of oxidative stress-related diseases, such as neurodegenerative and cardiovascular diseases, and provide additional protection against skin irritation, aging, CHD and cancer [Perrelli A et al., Oxidative Medicine and Cellular Longevity 2018, DOI: 10.1155/2018/6015351].

The antioxidant activity of Avns has been found to be 10 to 30 times higher than those of the typical cereal components ferulic acid, gentisic acid, phydroxybenzoic acid, protocagtechuic acid, syringic acid, vanillic acid and vanillin. The Avns differ in their antioxidant activity, Avn C having the highest activity, followed by Avn B and Avn A. Avns enriched oat extracts inhibit LDL oxidation in vitro. Both, animal studies and human clinical trials confirmed that oats antioxidants have the potential of reducing cardiovascular risks by lowering serum cholesterol, inhibiting LDL cholesterol oxidation and peroxidation. Another study has indicated that the consumption of oats and oats bran may reduce the risk of colon cancer not only because of their high fiber contents but also due to Avns. Furthermore, Avns enriched oat extracts have been shown to inhibit atherosclerosis and activation of the NF-kB transcription factor, which is the regulator of infection and inflammation [Hüseyin Boz, Phenolic Amides (Avenanthramides) in Oats—A Review, Czech J. Food Sci., 33, 2015 (5), 399-404].

Furthermore, avenanthramides are known to be beneficial in the treatment and prevention of itching or hearing loss or as 5-lipoxygenase inhbitors.

The anvenanthramide analogue dihydroavenanthramide D (CAS 697235-49-7, INCI name: hydroxyphenyl propamidobenzoic acid; the active ingredient in SymCalmin® provided by Symrise) is known for inhibiting mast cell degranulation and exhibiting anti-inflammatory effects through the interaction with the neurokinin-1 receptor.

Due to their potent beneficial biological activities, avenanthramides are valuable and highly interesting natural active ingredients for nutritional, cosmetic and health use for oral and/or topical applications for humans and animals.

On the other hand, there is an ongoing need in the cosmetics and pharmaceutical industry for the development of new substances or compositions, for use in skin and scalp protection and skin and scalp care and/or in the prevention and/or treatment of dermatoses, which have an improved cutaneous permeation of the active substances or compositions.

Cutaneous biological effects usually require the active compound to permeate into the skin.

The primary penetration barrier of the skin, the stratum corneum (SC), consists of corneocytes embedded in extracellular lipids.

In order to reach therapeutic drug concentrations, the stratum corneum has to be penetrated. By virtue of its structure and biochemical composition, the stratum corneum is selectively permeable. Generally speaking, small and moderately lipophilic molecules (having a molecular weight of less than 500 Da and a log P of 1 to 4) are likely to penetrate the skin well. Other drugs based on macromolecules which do not possess these physicochemical properties are primarily hindered by the skin's low permeability and usually require a suitable penetration enhancement strategy in order to penetrate the skin.

Skin penetration and permeation is a complex process with a variety of barriers to cross. Initially, the cosmetically or pharmaceutically active substance must dissolve in the customary preparation or formulation to enable it to diffuse inside the formulation to the SC interface. Then, the active substance diffuses passively out of the formulation and partitions into the barely permeable SC. It is known that penetration increases as log P increases. While lipophilic substances are favoured for penetrating the stratum corneum, diffusion through the stratum corneum will be the rate-limiting effect for most hydrophilic and amphiphilic substances. Because of the higher hydrophilicity of the deeper skin layers, an optimum hydrophilic-lipophilic balance is essential in order for active substances to penetrate dermally.

Numerous substances have been studied and are used for penetration enhancing activity. Many potential sites and modes of action have been identified for skin penetration enhancers.

Heuschkel S et al., European Journal of Pharmaceutics and Biopharmaceutics 72, 2009, 552-560, describes the influence of 1,2-alkanediol vehicles as penetration enhancer on the model drug dihydroavenenthramide D in a microemulsion.

Due to the complexity of skin penetration and permeation mechanisms on the one hand and the chemical and physicochemical properties of active substances or kind of formulation on the other hand does not allow an effective prediction of the effects of skin penetration enhancers on transdermal delivery. Additionally, the effect of one penetration enhancer compound in combination with one active substance cannot simply be generalised and applied to another, different active substance(s).

The aforementioned avenanthramides or preparations comprising an avenanthramide are hydrophilic substances and therefore exhibit poor permeation through the SC. Achieving therapeutic concentrations or topical bioavailability of the avenanthramide substances in the intended target is therefore highly challenging.

Accordingly, it is the object of the present invention to provide a composition or a customary preparation or formulation comprising an avenanthramide which exhibits enhanced skin penetration, in order to realize a high bioavailability of the active substance, i.e. avenanthramide or an analogue avenanthramide compound.

In particular, the aim of the present invention is to suggest a composition comprising one or more biodegradable, cosmetically or pharmaceutically well accepted, safe, easy-to-use and stable penetration enhancing substance(s) which is/are able to enhance the permeation of an avenanthramide or avenanthramides into the skin and which do not interfere with the beneficial biological activity of the avenanthramide(s) and the composition or the customary formulation properties as such.

Surprisingly, it turns out that the penetration of an avenanthramide through the skin can be significantly enhanced by adding a penetration enhancer.

### SUMMARY OF THE INVENTION

The aforementioned object is achieved in accordance with a first aspect of the present invention by providing a composition comprising or consisting of:
(i) at least one avenanthramide, selected from the group consisting of the avenanthramides A, B, C, D, E, F, G, H and L and mixtures of these avenanthramides; and
(ii) at least one penetration enhancer.

In a second aspect, the present invention relates to the use of said composition as a cosmetic, in particular for skin protectin and skin care, scalp protection and scalp care, hair care, nail care or in the prevention and/or treatment of skin conditions, intolerant or sensitive skin, skin irritation, skin reddening, wheals, pruritis (itching), skin aging, wrinkle formation, loss of skin volume, loss of skin elasticity, pigment spots, pigment abnormalities, or dry skin, i.e. for moisturising the skin.

In a third aspect, the present invention relates to the use of said composition as a medicament, in particular for use in the prevention and/or treatment of dermatological or keratological diseases, in particular dermatological diseases having a barrier related, inflammatory, immunoallergic, atherogenic, xerotic or hyperproliferative component or in the prevention and/or treatment of dermatological diseases accociated with increased ROS production.

In a fourth aspect, the present invention relates to the use of said composition for preparing cosmetic or pharmaceutical preparations/formulations.

Finally, the present invention relates to a method for preparing the composition according to the present invention.

The invention is specified in more detail in the appended claims. The invention itself, and its preferred variants, other objects and advantages, are however also apparent from the following detailed description in conjunction with the accompanying examples.

### DESCRIPTION OF FIGURES

FIG. 1 is a diagram showing the penetration behaviour of Avn A, Avn B, and Avn L in comparison to dihydroavenantramide D (DhAvn D) in the absence of a penetration enhancer.

### DETAILED DESCRIPTION OF THE INVENTION

The first main ingredient of the composition according to the first aspect of the present invention is at least one avenanthramide as active substance.

An active substance is the ingredient in a composition or preparation that is biologically active.

As used in this document, the phrase “at least one” means that the composition can comprise for example either one avenanthramide or more than one avenanthramide. Additionally, the phrase “at least one of”, when applied to a list, means anyone combination of the items specified in the list.

Within the context of the present invention, the general term “avenanthramide(s)” (anthranilic acid amides) is understood to mean inter alia a member of a group of phenolic alkaloids, i.e. naturally occurring avenanthramide(s), found mainly in oats (Avena sativa) but also present in white cabbage butterfly eggs (Pieris brassicae and P. rapae) and in fungus-infected carnations (Dianthus caryophyllus), as described in detail hereinafter, or non-naturally artificial produced avenanthramide analogue compound(s), as described in detail hereinafter.

The avenanthramides of the composition of the present invention are naturally found in and can be isolated and purified from oats. The two main species of oats are Avena sativa L. and Avena nuda L. (synonyms include Avena sativa subsp. nuda (L.) after Gillet & Magne, and Avena sativa var. nuda (L.) after Körn), wherein they appear to be most concentrated in the peripheral regions, husks, trichomes or straw. More than 50 distinct avenanthramides have been isolated from oat grains [Collins, Journal of Agricultural and Food Chemistry, 37 (1989), 60-66].

Avns can be represented by the following general Formula 1:

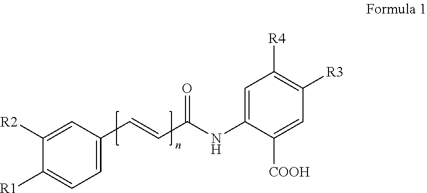

The following Table 1 shows examples of naturally occurring Avns based on general Formula 1.

TABLE 1

| Avenanthramide *) | CAS number | n | R1 | R2 | R3 | R4 |
|---|---|---|---|---|---|---|
| A | 108605-70-5 | 1 | OH | H | OH | H |
| B | 108605-69-2 | 1 | OH | OMe | OH | H |
| C | 116764-15-9 | 1 | OH | OH | OH | H |
| D | 115610-36-1 | 1 | OH | H | H | H |
| E | 93755-77-2 | 1 | OH | OMe | H | H |
| F | 116764-16-0 | 1 | OH | OH | H | H |
| G | 116764-17-1 | 1 | OH | H | H | OH |
| H | 116764-18-2 | 1 | OH | OMe | H | OH |
| K | 116764-19-3 | 1 | OH | OH | H | OH |
| X | 1158480-77-3 | 1 | OH | H | OH | OMe |
| Y (2 **) | 154992-25-3 | 1 | OH | OMe | OH | OMe |
| Z | 1158480-80-8 | 1 | OH | OH | OH | OMe |
| AA | 157799-28-5 | 1 | OH | H | OH | OH |
| BB | 2304718-64-5 | 1 | OH | OMe | OH | OH |
| CC | 1819995-77-1 | 1 | OH | OH | OH | OH |
| O (L **) | 172549-38-1 | 2 | OH | H | OH | H |
| P | 1358438-37-5 | 2 | OH | OMe | OH | H |
| Q | 2227208-43-5 | 2 | OH | OH | OH | H |
| L | 2301866-39-5 | 2 | OH | H | H | H |
| M | 101618-11-5 | 2 | OH | OMe | H | H |
| N | 101618-21-7 | 2 | OH | OH | H | H |
| R | 1191042-39-3 | 2 | OH | H | H | OH |
| S | 2301866-43-1 | 2 | OH | OMe | H | OH |
| T | 2301864-63-9 | 2 | OH | OH | H | OH |
| u | 2301864-86-6 | 2 | OH | H | OH | OMe |
| V | 2304718-63-4 | 2 | OH | OMe | OH | OMe |
| W | 2304718-62-3 | 2 | OH | OH | OH | OMe |
| 00 | 2301866-28-2 | 2 | OH | H | OH | OH |
| PP | 2301864-57-1 | 2 | OH | OMe | OH | OH |
| QQ | 2301864-89-9 | 2 | OH | OH | OH | OH |

*) Abbreviations Collins [de Bruijn et al., Food Chemistry (2018), doi: https://doi.Org/10.1016/j.foodchem.2018.11.013, supplementary information Table S1]
**) More commonly used, non-Collins abbreviations

A number of studies have demonstrated that avenanthramides have anti-inflammatory, anti-oxidant, anti-itch, anti-irritant and anti-atherogenic activities.

The naturally occurring single avenanthramides or mixtures of avenanthramides as described above, are obtained and isolated and/or fractionated from the plant of the genus Avena by extraction, in particular from any oat species, fresh or dried, or parts thereof, such as milled grains, non-milled grains, husks, trichomes or oat straw of the oat species Avena sativa or Avena nuda.

The extracting solvent (extractant) for favourably extracting the avenanthramide compound(s) as used according to the present invention is selected from the group consisting of mixtures of water and an organic solvent, wherein the organic solvent is preferably a solvent suitable for foodstuffs or cosmetic or pharmaceutical preparations. It goes without saying that such solvents need be suitable for and compatible with the preparation of foods, cosmetics or pharmaceutical preparations.

In a more preferred variant, the extracting solvent comprises a mixture of water and an alcohol or acetone. The alcohol is preferably selected from the group consisting of methanol, ethanol, n-propanol, isopropanol, n-butanol, isobutanol, t-butanol and mixtures, i. e. combinations, thereof. The most preferred extracting solvents (extractant) for the extraction step of the present invention are methanol, ethanol, n-propanol, isopropanol or acetone or any mixtures respective combinations of said solvents, each in mixture with water. The use of pure organic solvents is not advantageous, due to the co-extraction of triglycerides.

The mixing ratio of water to the organic solvent, preferably water to the alcohol or water to acetone, in the extracting solvent is in a range of 10:90 to 90:10 (v/v), preferably in a range of 20:80 to 80:20 (v/v) and most preferably in a range of 30:70 to 70:30 (v/v), based in each case on the resulting extracting solvent.

Particularly preferred extracting solvents (extractants) are: methanol/water (3:7), methanol/water (1:1), methanol/water (7:3), ethanol/water (3:7), ethanol/water (1:1), ethanol/water (1:4), ethanol/water (7:3), isopropanol/water (3:7), isopropanol/water (1:1), isopropanol/water (7:3), aceton/water (3:7), aceton/water (1:1), aceton/water (7:3).

In order to improve the extraction yield, the oat source is extracted at a temperature ranging from 30 to 80° C., preferably from 40 to 70° C. and more preferably from 50 to 60° C. The extraction yield for milled oat grains increases with increasing temperatures between 40 and 70° C.

Apart from avenanthramide compounds isolated and purified from natural sources, the naturally occurring single avenanthramides can be produced by organic synthesis. Methods of synthesis known in the art are illustrated for example in U.S. Pat. Nos. 6,096,770 and 6,127,392, Japanese Patent No. J60019 754 A and Hungarian Patent No. HU 200 996 B.

Said synthetic prepared avenanthramide substances are identical to the corresponding naturally occurring avenanthramide compounds as extracted and isolated and/or from oats.

According to the present invention, the definition of the avenanthramide compound(s) does not encompass the analogue compound dihydroavenanthramide D (DhAvn D) (2-(3-(4-hydroxyphenyl-)propanoyl-)amidobenzoic acid), represented by Formula 3 below,

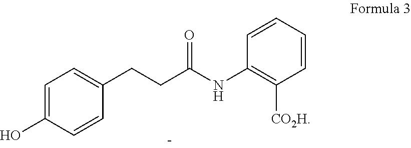 [SMILES: O=C(CCc1ccc(O)cc1)Nc1ccccc1C(=O)O]

Thus, dihydroavenanthramide D (DhAvn D) is excluded from the above avenanthramide definition.

The analogue avenanthramide compound dihydroavenanthramide D (2-(3-(4-hydroxyphenyl-)propanoyl-)amidobenzoic acid), represented by Formula 3, is less preferred.

Besides the above natural occurring avenanthramides, novel avenanthramide analogues have been produced in recombinant yeast, including N-(4′-hydroxycinnamoyl)-3-hydroxyanthranilic acid (YAvn I) and

N-(3′-4′-dihydroxycinnamoyl)-3-hydroxyanthranilic acid (YAvn II), which were generated by engineering a Saccharomyces cerevisiae strain with two plant genes (4cl-2 from tobacco and hct from globe artichoke) encoding key proteins involved in the biosynthesis of phenolic esters. Remarkably, YAvn I and YAvn II share structural similarities with Avn A and Avn C, respectively.

Generally, the consumers are aware of the difference between natural and artificial prepared compounds. In the context of the present invention naturally occurring avenanthramides obtained from naturally sources or naturally occurring avenanthramides produced synthetically are preferred and are used likewise. Nonetheless, non-naturally occurring avenanthramide can also be important.

The term “avenanthramide” is intended to also include their various isomers that exist, notably the naturally occurring trans-isomers as well as the cis-isomers, such as avenanthramides with cis-isomerized double bond (Formula 1 or 2 with n=1) or 1 or 2 cis-isomerized double bonds (Formula 1 or 2 with n=2) induced e.g. by photoisomerization due to light exposure.

In particular, within the context of the present invention, the avenanthramide is any one of the avenanthramide compounds represented by the general Formula 1 and defined in Table 1 or any isomer thereof as described above.

In a preferred variant of the present invention according to the first aspect, the composition comprises at least one avenanthramide selected from the group consisting of avenanthramides A, B, C, G, H, K, L, R and mixtures thereof. In another variant of the present invention, the composition comprises at least one of the avenanthramides selected from the group consisting of A, B, C, L and mixtures thereof is more preferred Most preferred is a composition which comprises at least one of the avenanthramides selected from the group consisting of A, B, L and mixtures thereof.

Specifically preferred is a composition which comprises the avenanthramide A, or a composition which comprises the avenanthramide B, or a composition which comprises the avenanthramide C, or a composition which comprises the avenanthramide G, or a composition which comprises the avenanthramide H, or a composition which comprises the avenanthramide K, or a composition which comprises the avenanthramide L, or a composition which comprises the avenanthramide R.

In another variant, the composition of the present invention comprises a mixture of two, three, four or even more different avenanthramides selected from the group consisting of avenanthramides A, B, C, G, H, K, L (non-Collins abbreviations; CAS number 172549-38-1) (also called O or 2pd) and R. The combinations or mixtures of avenanthramides can thus include any one of the following combinations of avenanthramides:

### A/B; A/C; A/G; A/H; A/K; A/L; A/R;

### B/C; B/G; B/H; B/K; B/L; B/R;

### C/G; C/H; C/K; C/L; C/R;

### G/H; G/K; G/L; G/R;

### H/K; H/L; H/R;

### K/L; K/R; and

### L/R;

### A/B/C; A/B/G; A/B/H; A/B/K; A/B/L; A/B/R; A/C/G; A/C/H; A/C/K; A/C/L; A/C/R; A/G/H;

### A/G/K; A/G/L; A/G/R; A/H/K; A/H/L; A/H/R; A/K/L; A/K/R; A/L/R; B/C/G; B/C/H; B/C/K;

### B/C/L; B/C/R; C/G/H; C/G/K, C/G/L; C/G/R; G/H/K; G/H/L; G/H/R; H/K/L; H/K/R; K/L/R;

### A/B/C/G; A/B/C/H; A/B/C/K; A/B/C/L; A/B/C/R; A/C/G/H; A/C/G/K; A/C/G/L; A/C/G/R;

### A/G/H/K; A/G/H/L; A/G/H/R; A/H/K/L; A/H/K/R; A/K/L/R; B/C/G/H; B/C/G/K; B/C/G/L;

### B/C/G/R; C/G/H/K; C/G/H/L; C/G/H/R; G/H/K/L; G/H/K/R and H/K/L/R.

The most preferred mixtures of avenanthramides according to the present invention are however A/B, A/C, A/L, B/C, B/L, A/B/C, A/B/L and A/C/L.

In addition to the above avenanthramide compounds or avenanthramide combinations, the composition can further comprise one or more avenanthramide(s) other than the avenanthramides A, B, C, G, H, K, L (non-Collins abbreviations; CAS number 172549-38-1) (also called O or 2pd) and R, such as avenanthramides D, E, F U, X, Y (also termed 2), AA, CC or OO or any of the remaining avenanthramide compounds specified in Table 1.

The at least one avenanthramide is added to the composition according to the first aspect of the present invention either as a single avenanthramide compound isolated and/or fractionated from natural sources, such as oat, or obtained by organic synthesis, as described above, i. e. as a single avenanthramide A, B, C, G, H, K, L or R compound, or as a mixture of two or more of said single avenanthramide compounds.

Alternatively, the at least one avenanthramide is added to the composition according to the first aspect of the present invention in the form of an oat extract fraction, comprising at least one avenanthramide A, B, C, G, H, K, L or R compound or a mixture thereof.

In a further alternative, the at least one anvenanthramide is added to the composition as a single avenanthramide compound as described before in combination with an oat extract fraction, comprising at least one avenanthramide A, B, C, G, H, K, L or R compound or a mixture thereof.

The concentration of the avenanthramide, i. e. the active substance or active ingredient, in the composition can vary a great deal and will depend on a variety of factors, including the disease or condition to be treated, the nature and activity of the active substance or active ingredient, the desired effect, and the ability and speed of the active substance or active ingredient to reach its intended target.

The at least one avenanthramide or mixture of avenanthramides, as described above, may be present in the composition at a concentration or total amount of 0.0001 to 5.0 wt %, based on the total weight of the composition. In a preferred variant, the composition comprises the at least one avenanthramide or mixture of avenanthramides at a concentration or total amount of 0.0005 to 2.0 wt %, still more preferred at a concentration or total amount of 0.001 to 1.0 wt %, based on the total weight of the composition.

The above specified avenanthramides are hydrophilic substances and therefore exhibit poor permeation through the SC. Avn A<Avn B<Avn L have a weaker penetration behaviour than dehydroavenanthramide D (DhAvn D). However, Avn C penetrates better than dehydroavenanthramide D (DhAvn D).

The second main ingredient of the composition according to the first aspect of the present invention is at least one penetration enhancer. “Penetration enhancer” means a substance used to modify (usually, increase or accelerate) the rate of permeation, into the skin (cutaneous delivery) or through the skin into another tissue (transdermal) and into the body, of one or more active substance(s) or active ingredient(s) in a composition, in order to achieve therapeutic concentrations or topical bioavailability of said substance(s) in the intended target.

Within the context of the present invention, the term “at least one” is intended to include either one penetration enhancer only or more than one penetration enhancer, i. e. two, three or even more different penetration enhancers.

“Cutaneous active delivery” refers to administering one or more than one active ingredient(s) or active substance(s) to the surface of an individual's skin such that the active ingredient(s) or active substance(s) penertrate(s) into the skin (epidermis and/or dermis) and delivers there its efficacy and benefit.

“Transdermal drug delivery” or “transdermal drug administration” refers to administering a drug to the surface of an individual's skin such that the drug passes through the skin tissue and into the individual's blood stream. The term “transdermal” is intended to include “transmucosal” drug administration, i. e. administering a drug to the mucosal (such as for example sublingual, buccal, vaginal or rectal) surface of an individual such that the drug passes through the mucosal tissue and into the individual's blood stream.

“Topical drug delivery” or “topical drug administration” are used here in their conventional sense to mean delivering a topical drug of a pharmacologically active agent to the skin or mucosa, as for example in the treatment of various skin disorders. Topical drug administration, as opposed to transdermal administration, is often used to provide a local rather than systemic effect.

Transdermal drug delivery can be used to circumvent first-pass metabolism and provide sustained drug release for a prolonged period of time. Topical drug delivery allows a drug to be applied directly to the surface area to be treated, which can be useful in localising the treatment and minimising side effects. Skin, however, which has evolved to impede the influx of toxins into the body, offers a very low permeability to the movement of foreign molecules across it. The stratum corneum is responsible for this barrier. It possesses a unique hierarchical structure of a lipid-rich matrix with embedded keratinocytes in the upper strata (15 μm) of the skin (Bouwstra 1997). Overcoming this barrier safely and reversibly is a fundamental problem that persists even today in the field of transdermal or topical delivery.

One well-known approach is to use substances or chemicals, that can enhance or accelerate the passage of the active substance or the active ingredient through the stratum corneum by altering the state of the lipids in the stratum corneum or by increasing the solubility of the active substance or active ingredient in the stratum corneum.

The ability of a substance to accelerate stratum corneum permeation through the skin barrier is dependent on the physicochemical properties (such as melting point, molecular weight, molecular geometry, charge, lipophilicity, etc.) of the active substance. For new active substances, studies may need to be conducted to determine the optimum choice of penetration enhancer and/or complete composition or preparation in order to achieve the desired dermal delivery.

Although numerous substances have been studied for penetration enhancing activity, including sulphoxides (such as dimethyl sulphoxide, DMSO), azones (such as laurocapram), pyrrolidones (for example 2-pyrrolidone, 2P), alcohols and alkanols (ethanol, or decanol), surfactants and terpenes, only a handful are actually used in practice. Many potential sites and modes of action have been identified for skin penetration enhancers.

Even though whole libraries of chemical penetration enhancers exist, the complexity of skin penetration and permeation mechanisms on the one hand and the chemical and physicochemical properties of active substances on the other does not allow an effective prediction of the effects of skin penetration enhancers on cutaneous or transdermal delivery, and the effect of one penetration enhancer compound in combination with one active substance cannot simply be generalised and applied to another, different active substance.

Surprisingly, it turns out that combining with a penetration enhancer selected from the group consisting of diols, polyols, alcohols, dimethyl isosorbide (INCI), triethyl citrate, butylene carbonate, glycerine carbonate, dipropylene glycol or any mixtures of these is beneficial in significantly improving the penetration and permeation of avenanthramide(s) into the skin. This effect is demonstrated by the following examples. Thus, by increasing the penetration and permeation into the skin, the bioavailability of the active substance(s), i. e. avenanthramide(s), in the intended target can be increased.

A diol is a chemical compound containing two hydroxyl groups. In a preferred variant, the diol is selected from the group of straight-chain 1,2-alkanediols having 3 to 12 C atoms, preferably 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, 1,1′-oxydi-2-propanol (dipropylene glycol) and its isomers, and any mixtures of these.

A polyol is an organic compound containing multiple hydroxyl groups. In a preferred variant, the polyol is selected from the group consisting of 1,2,3-propanetriol (glycerine), sorbitol, fructose, maltose, and trehalose.

In a preferred variant of the composition according to the present invention, the penetration enhancer is selected from the group consisting of diols, preferably straight-chain 1,2-alkanediols having 3 to 12 C atoms, in particular 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecane diol, 1,3-butanediol (butylene glycol), 1,3-propanediol, 1,4-butanediol, 1,1′-oxydi-2-propanol (dipropylene glycol) and its isomers; polyols, preferably sorbitol, fructose, maltose, trehalose; alcohols; dimethyl isosorbide (INCI); triethyl citrate; butylene carbonate; glycerine carbonate; dipropylene glycol or any mixtures of these.

Of the aforementioned penetration enhancers, straight-chain 1,2-alkanediols having 3 to 12 C atoms and glycerine are preferred. Of the 1,2-alkanediols, 1,2-pentanediol, 1,2-hexanediol and 1,2-octanediol are particularly preferred, as demonstrated by the following examples, and 1,2-hexanediol is the single most preferred of these.

A mixture of 1,2-hexanediol and 1,2-octanediol was even more efficient, even if the blend of penetration enhancers is used in a lower concentration, as it is demonstrated by the following examples.

Additionally, as it is demonstrated in the following examples, 1,2-hexanediol and a blend of 1,2-hexanediol and 1,2-octanediol performed better than 1,2-pentanediol, even in lower concentrations.

According to the present invention, the penetration enhancer does not encompass glycerine. Thus, glycerine is excluded from the above penetration enhancer definition.

The penetration enhancer glycerine is less preferred.

In a preferred variant, the composition according to the present invention does not contain glycerin as penetration enhancer.

Since the 1,2-alkanediols are known to possess skin-moisturising properties, the use of such a 1,2-alkanediol penetration enhancer provides additional benefits to skin care and scalp care preparations aimed at relieving itching, since itchy skin is often accompanied by dry, sensitive or damaged skin.

A combination of the penetration enhancers 1,2-hexanediol and 1,2-octanediol also enhances the performance of preservatives in the end preparations, allowing compositions to have reduced preservative levels which is in particular beneficial for itchy skin associated with dry, sensitive or damaged skin.

The amount of the penetration enhancer present in the composition according to the present invention can be between 0.01 and 10.0 wt %, based on the total weight of the composition. In a preferred variant, the concentration of penetration enhancer in the composition is 0.5 to 7.0 wt %, based on the total weight of the composition, and can even more preferably be between 0.1 and 5.0 wt %, based on the total weight of the composition.

In a preferred variant in the composition according to the present invention, the penetration enhancers are used in the following concentrations ranges, depending on their different penetration activity: C5 diols as defined above in a concentration of 0.5 to 6 wt %, or C6 diols as defined above in a concentration of 0.2 to 3 wt %, or C7 diols as defined above in a concentration of 0.1 to 2 wt %, or C8 diols as defined above in a concentration of 0.1 to 1 wt %, or C10 diols as defined above in a concentration of 0.01 to 0.5 wt %, each based on the total weight of the composition.

As demonstrated by the following examples, adding one of the above penetration enhancers considerably enhances the penetration of avenanthramides A, B, C, G, H, K, L, or R. Preferably, adding one of the above penetration enhancers significantly improves the penetration of one of the avenanthramides A, B, C or L. More preferably, adding one of the above penetration enhancers significantly improves the penetration of one of the avenanthramides A, B or L. The modulation of permeation for avenanthramide A is in a range of 15% to 170%, preferably 15% to 65%, for avenanthramide B in a range of preferably 35% to 95%, and for avenanthramide L in a range of preferably 115% to 170%, depending on the concentration of the penetration enhancer.

Due to the differing retention of various cosmetically or pharmaceutically active substances in the composition according to the present invention, i. e. various avenanthramides, in the stratum corneum of the skin exposed to the composition, each composition will require a different composition of penetration enhancers.

A preferred variant of the composition according to the present invention comprises a combination of two or even more penetration enhancers. Combining two or even more penetration enhancers allows the penetration of different cosmetically or pharmaceutically active substances in the composition to be selectively improved. If the composition comprises two different avenanthramides having different structures, two different penetration enhancers can be combined in the composition, one of which has an improved effect for the first avenanthramide and the other of which has an improved effect for the second avenanthramide, wherein the concentration of each enhancer required to achieve the desired enhancement may be lower than the concentration required when either of the enhancers is used individually.

In a preferred variant, the composition according to the first aspect of the present invention includes any one of the following combinations:
Avn A in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, 1,1′-oxydi-2-propanol (dipropylene glycol), or glycerine;
Avn B in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, 1,1′-oxydi-2-propanol (dipropylene glycol), or glycerine;
Avn C in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, 1,1′-oxydi-2-propanol (dipropylene glycol), or glycerine;
Avn G in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, 1,1′-oxydi-2-propanol (dipropylene glycol), or glycerine;
Avn H in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, 1,1′-oxydi-2-propanol (dipropylene glycol), or glycerine;
Avn K in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, 1,1′-oxydi-2-propanol (dipropylene glycol), or glycerine;
Avn L in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, 1,1′-oxydi-2-propanol (dipropylene glycol), or glycerine; or
Avn R in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, 1,1′-oxydi-2-propanol (dipropylene glycol), or glycerine.

In a more preferred variant, the composition according to the first aspect of the present invention includes any one of the following combinations:
Avn A in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, or 1,1′-oxydi-2-propanol (dipropylene glycol);
Avn B in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, or 1,1′-oxydi-2-propanol (dipropylene glycol);
Avn C in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, or 1,1′-oxydi-2-propanol (dipropylene glycol);
Avn G in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, or 1,1′-oxydi-2-propanol (dipropylene glycol);
Avn H in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, or 1,1′-oxydi-2-propanol (dipropylene glycol);
Avn K in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, or 1,1′-oxydi-2-propanol (dipropylene glycol);
Avn L in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, or 1,1′-oxydi-2-propanol (dipropylene glycol); or
Avn R in combination with one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-butanediol (butylene glycol), 1,3-propanediol, 1,4-butanediol, or 1,1′-oxydi-2-propanol (dipropylene glycol).

Particularly favourable is a combination including
Avn A in combination with one or more of 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-propanediol, or 1,1′-oxydi-2-propanol (dipropylene glycol);
Avn B in combination with one or more of 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-propanediol, or 1,1′-oxydi-2-propanol (dipropylene glycol);
Avn C in combination with one or more of 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-propanediol, or 1,1′-oxydi-2-propanol (dipropylene glycol);
Avn G in combination with one or more of 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-propanediol, or 1,1′-oxydi-2-propanol (dipropylene glycol);
Avn H in combination with one or more of 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-propanediol, or 1,1′-oxydi-2-propanol (dipropylene glycol);
Avn K in combination with one or more of 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-propanediol, or 1,1′-oxydi-2-propanol (dipropylene glycol);
Avn L in combination with one or more of 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-propanediol, or 1,1′-oxydi-2-propanol (dipropylene glycol); or
Avn R in combination with one or more of 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecanediol; 1,3-propanediol, or 1,1′-oxydi-2-propanol (dipropylene glycol).

In a particularly preferred variant, the composition according to the first aspect of the present invention includes any one of the following combinations:
Avn A in combination with one or more of 1,2-pentanediol (Hydrolite-5), 1,2-hexanediol (Hydrolite-6), 1,2-heptanediol, 1,2-octanediol, or 1,2-nonanediol;
Avn B in combination with one or more of 1,2-pentanediol (Hydrolite-5), 1,2-hexanediol (Hydrolite-6), 1,2-heptanediol, 1,2-octanediol, or 1,2-nonanediol;
Avn C in combination with one or more of 1,2-pentanediol (Hydrolite-5), 1,2-hexanediol (Hydrolite-6), 1,2-heptanediol, 1,2-octanediol, or 1,2-nonanediol;
Avn G in combination with one or more of 1,2-pentanediol (Hydrolite-5), 1,2-hexanediol (Hydrolite-6), 1,2-heptanediol, 1,2-octanediol, or 1,2-nonanediol;
Avn H in combination with one or more of 1,2-pentanediol (Hydrolite-5), 1,2-hexanediol (Hydrolite-6), 1,2-heptanediol, 1,2-octanediol, or 1,2-nonanediol;
Avn K in combination with one or more of 1,2-pentanediol (Hydrolite-5), 1,2-hexanediol (Hydrolite-6), 1,2-heptanediol, 1,2-octanediol, or 1,2-nonanediol;
Avn L in combination with one or more of 1,2-pentanediol (Hydrolite-5), 1,2-hexanediol (Hydrolite-6), 1,2-heptanediol, 1,2-octanediol, or 1,2-nonanediol; or
Avn R in combination with one or more of 1,2-pentanediol (Hydrolite-5), 1,2-hexanediol (Hydrolite-6), 1,2-heptanediol, 1,2-octanediol, or 1,2-nonanediol.

In a particularly preferred variant, the composition according to the first aspect of the present invention includes any one of the following combinations:
Avn A in combination with 1,2-heptanediol;
Avn B in combination with 1,2-heptanediol;
Avn C in combination with 1,2-heptanediol;
Avn G in combination with 1,2-heptanediol;
Avn H in combination with 1,2-heptanediol;
Avn K in combination with 1,2-heptanediol;
Avn L in combination with 1,2-heptanediol; or
Avn R in combination with 1,2-heptanediol.

Due to a synergistic effect, in a most preferred variant, the composition according to the first aspect of the present invention includes any one of the following combinations:
Avn A in combination with a blend of 1,2-hexanediol plus 1,2-octanediol;
Avn B in combination with a blend of 1,2-hexanediol plus 1,2-octanediol;
Avn C in combination with a blend of 1,2-hexanediol plus 1,2-octanediol;
Avn G in combination with a blend of 1,2-hexanediol plus 1,2-octanediol;
Avn H in combination with a blend of 1,2-hexanediol plus 1,2-octanediol;
Avn K in combination with a blend of 1,2-hexanediol plus 1,2-octanediol;
Avn L in combination with a blend of 1,2-hexanediol plus 1,2-octanediol; or
Avn R in combination with a blend of 1,2-hexanediol plus 1,2-octanediol.

In a further most preferred variant, the composition according to the first aspect of the present invention includes any one of the following combinations:
Avn A in combination with a blend of 1,2-pentanediol plus 1,2-heptanediol;
Avn B in combination with a blend of 1,2-pentanediol plus 1,2-heptanediol;
Avn C in combination with a blend of 1,2-pentanediol plus 1,2-heptanediol;
Avn G in combination with a blend of 1,2-pentanediol plus 1,2-heptanediol;
Avn H in combination with a blend of 1,2-pentanediol plus 1,2-heptanediol;
Avn K in combination with a blend of 1,2-pentanediol plus 1,2-heptanediol;
Avn L in combination with a blend of 1,2-pentanediol plus 1,2-heptanediol; or
Avn R in combination with a blend of 1,2-pentanediol plus 1,2-heptanediol.

In a further most preferred variant, the composition according to the first aspect of the present invention includes any one of the following combinations:
Avn A in combination with a blend of 1,2-hexanediol plus 1,2-heptanediol;
Avn B in combination with a blend of 1,2-hexanediol plus 1,2-heptanediol;
Avn C in combination with a blend of 1,2-hexanediol plus 1,2-heptanediol;
Avn G in combination with a blend of 1,2-hexanediol plus 1,2-heptanediol;
Avn H in combination with a blend of 1,2-hexanediol plus 1,2-heptanediol;
Avn K in combination with a blend of 1,2-hexanediol plus 1,2-heptanediol;
Avn L in combination with a blend of 1,2-hexanediol plus 1,2-heptanediol; or
Avn R in combination with a blend of 1,2-hexanediol plus 1,2-heptanediol.

In a further most preferred variant, the composition according to the first aspect of the present invention includes any one of the following combinations:
Avn A in combination with a blend of 1,2-octanediol plus 1,2-heptanediol;
Avn B in combination with a blend of 1,2-octanediol plus 1,2-heptanediol;
Avn C in combination with a blend of 1,2-octanediol plus 1,2-heptanediol;
Avn G in combination with a blend of 1,2-octanediol plus 1,2-heptanediol;
Avn H in combination with a blend of 1,2-octanediol plus 1,2-heptanediol;
Avn K in combination with a blend of 1,2-octanediol plus 1,2-heptanediol;
Avn L in combination with a blend of 1,2-octanediol plus 1,2-heptanediol; or
Avn R in combination with a blend of 1,2-octanediol plus 1,2-heptanediol.

Particularly advantageous is a combination of avenanthramide B plus a blend of 1,2-hexanediol and 1,2-octancediol, or a combination of avenanthramide L plus 1,2-pentanediol (Hydrolite-5) or 1,2-hexanediol (Hydrolite-6).

For example, a mixture of 1,2-hexanediol and 1,2-octanediol was more effective when used at 1% than was 1,2-hexanediol when used alone at the same dosage, as it enhanced the permeation of avenanthramide B by 87% as compared to 62%, as it is demonstrated by the following example.

Particularly preferred mixtures according to the present invention are those in which the composition comprises or consists of:
0.0001 to 5.0 wt % of the at least one avenanthramide, selected from the group consisting of the avenanthramides A, B, C, D, E, F, G, H and L and mixtures of these avenanthramides, preferably 0.0005 to 2.0 wt %, even more preferably 0.001 to 1 wt %; and
0.01 to 10.0 wt % of the at least one penetration enhancer, preferably 0.5 to 7.0 wt %, even more preferably 0.1 to 5.0 wt %,
   based on the total weight of the composition.

The best penetration effect is obtained, if in the composition according to the present invention, the concentration of the penetration enhancer is significantly higher than the concentration of the at least one avenanthramide or an analogue thereof. Particularly preferred mixtures according to the first aspect of the present invention are those in which the weight ratio of the total amount of the avenanthramide(s) to the total amount of the penetration enhancer(s) is between 1:100 and 1:1, preferably between 1:50 and 1:1 and particularly preferably between 1:20 and 1:1.

The compositions according to the invention, in particular those characterised as preferred compositions, possess a synergistically intensified skin penetration efficacy. The efficacy of the composition is surprisingly superior to that of compositions comprising one or more avenanthramide(s) only. Additionally, the penetration effect for an avenanthramide A, B, C, D, E, F, G, H, L or mixtures thereof in combination with a penetration enhancer is even better than for dihydroavenanthramide D in combination with a penetration enhancer as it is demonstrated by the following examples.

The cosmetically or pharmaceutically active substances, i. e. avenanthramide(s) as specified above, which exhibit biological benefits of great interest, such as anti-inflammatory, antioxidant, anti-itching, anti-irritant and anti-atherogenic activities, can better penetrate the stratum corneum and thus better reach their intended target. The composition according to the present invention is thus beneficial for skin and scalp care or skin and scalp protection and in the prevention and/or treatment of dermatoses.

The compositions according to the present invention are also particularly effective and free of any toxicologically or dermatologically critical secondary components; they can therefore be used without further concerns in cosmetic or pharmaceutical preparations.

It should generally be borne in mind that the substances to be used in the composition and in the end preparation should be
toxicologically acceptable,
well tolerated by the skin,
stable (in particular in the customary formulations),
preferably odourless and
able to be produced inexpensively (i.e. using standard processes and/or starting from standard precursors)
   in the concentration range relevant to activity and administration.

Due to their aforementioned superior anti-inflammatory, anti-oxidant, anti-itch, anti-irritant and anti-antherogenic activities of the avenanthramides in combination with the above described properties of the penetration enhancers, the composition according to the present invention is thus beneficial for skin and scalp protection and skin and scalp care and in the prevention and/or treatment of dermatoses.

Another aspect of the present invention therefore relates to the use of the composition according to the first aspect of the present invention as a cosmetic, in particular for skin protection and skin care, scalp protection and scalp care, hair care, nail careor for use in the prevention and/or treatment of skin conditions, intolerant or sensitive skin, skin irritation, skin reddening, wheals, pruritus (itching), skin aging, wrinkle formation, loss of skin volume, loss of skin elasticity, pigment spots, pigment abnormalities, dry skin, i.e. for moisturising the skin.

Another aspect of the present invention relates to the composition according to the first aspect of the present invention for use as a medicament.

Due to its aforementioned superior properties, the composition according to the first aspect of the present invention is particularly useful in the prevention and/or treatment of dermatological or keratological diseases, in particular dermatological or keratological diseases having a barrier related, inflammatory, immunoallergic, atherogenic, xerotic or hyperproliferative component.

Examples of such dermatological or keratological disorders include atopic dermatitis (neurodermitis), psoriasis, acneiform exanthema, sebostasis, xerosis, eczema, hyper seborrhea and hypo seborrhea, dermatitis, rosacea, erythema, pruritus, inflammation, irritation, fibrosis, Lichen planus, Pityriasis rosea, Pityriasis versicolor, autoimmune bullous diseases, urticaria, angioedema, allergic skin reactions, wound healing, and tissue regeneration.

In a particularly preferred variant, the composition comprising at least one avenanthramide or an analogue thereof according to the present invention, is beneficial useful in the prevention and/or treatment of pruritis (itching).

Chronic pruritis is a common symptom associated with various dermatological conditions and systemic diseases, with no known underlying condition in some cases. Chronic pruritis is classified by clinical presentation (for example, association with diseased/inflamed or normal/non-inflamed skin and/or presence of secondary scratch lesions) and underlying causes (of for example dermatological, systemic, neurological, psychosomatic, mixed or undetermined origin).

Due to the particular antioxidative effect of the avenanthramide(s), the present invention also relates to the composition according to the first aspect of the present invention for use in the prevention and/or treatment of dermatological diseases associated with increased ROS production and/or wherein the skin diseases associated with increased ROS production are selected from the group consisting of atopic dermatitis, neurodermitis, psoriasis, rosacea, acneiform eruptions, sebostasis and xerosis.

The use of an avenanthramide or an analogue thereof for these respective purposes corresponds to a method for imparting the respective therapeutic activity of the substance by adding a therapeutically effective amount of the substance or preparation.

Within the context of the present invention, an effective amount of a composition is the amount of each active component, i. e. an avenanthramide, that is sufficient to show a benefit, such as a reduction in a symptom associated with the disorder, disease or condition to be treated. When applied to a combination or a preparation, as in the present case, the term refers to the amount of the combined active substances or active ingredients resulting in the benefit.

Accordingly, the present invention relates to a method for treating dermatological or keratological diseases in a subject in need thereof, wherein the method comprises administering the subject with a therapeutically effective amount of a composition comprising or consisting of: at least one avenanthramide or an analogue thereof and at least one penetration enhancer in an amount which is sufficient for the prevention and/or treatment of dermatological or keratological diseases.

Another aspect of the present invention relates to the use of the composition according to the first aspect of the present invention for preparing cosmetic or pharmaceutical preparations for skin care, scalp care, nail care and hair care and/or in the prevention and/or treatment of said skin conditions and/or in the prevention and/or treatment of said dermatological or keratological disorders.

In a preferred variant, the cosmetic or pharmaceutical preparations according to the present invention comprise the composition according to the present invention in an amount of 0,0001 to 10.0 wt %, more preferred 0.0005 to 5 wt %, most preferred 0.001 to 1 wt %, based on the total weight of the preparation.

The composition according to the first aspect of the present invention can be easily incorporated into conventional cosmetics or pharmaceuticals.

Within this context, the cosmetic and/or pharmaceutical preparation containing the composition according to the present invention can be conventional in composition and serve to treat the skin, scalp, hair and/or nails within the context of a dermatological or keratological treatment or cosmetic care.

The composition according to the present invention can be combined and used with a large number of other components, optionally even synergistically intensifying or supplementary substances, in order to produce preferred cosmetic and/or pharmaceutical preparations or products such as active substances or cosmetically or pharmaceutically acceptable excipients.

An “active substance” means a substance or compound that imparts a primary utility to a composition or formulation. Examples of such active substances include antioxidants, preservatives, (metal) chelating agents, penetration enhancers, anti-inflammatories, antibacterial or antimycotic substances, substances having a reddening-alleviating or itch-alleviating action, lenitive substances or moisturisers.

An “excipient” refers to an inactive substance used to formulate cosmetics or pharmaceuticals as a result of processing or manufacture.

Since dermatological conditions or diseases are often associated with dry skin, scratched skin, skin lesions or even inflammation, the composition or cosmetic and/or pharmaceutical preparation, comprising the compositon according to the present invention, particularly advantageously contains a skin-moisturising and/or moisture-retaining substance, a cooling agent, an osmolyte, a keratolytic substance, a nurturing substance, an anti-inflammatory, antibacterial or antimycotic substance and/or a substance having a reddening-alleviating or itch-alleviating action and/or a lenitive substance and/or a moisturiser.

Itching occurs with particular intensity when the skin is dry. The use of skin-moisturising and/or moisture-retaining substances in cosmetic and/or pharmaceutical preparations can significantly alleviate itching. The composition or cosmetic and/or pharmaceutical preparation according to the present invention can therefore also be particularly advantageously combined with one or more skin-moisturising and/or moisture-retaining substances. The composition or cosmetic and/or pharmaceutical preparation according to the present invention can therefore advantageously also contain the following moisturising and/or moisture-retaining substances: sodium lactate, urea, urea derivatives, alcohols, glycerol, diols such as propylene glycol, hexylene glycol, 1,2-pentanediol, 1,2-hexanediol, 1,2-heptanediol, 1,2-octanediol, 1,2-nonanediol, 1,2-decanediol or mixtures of said diols, in particular mixtures of 1,2-hexanediol and 1,2-octanediol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulphate, lanolin, lanolin esters, amino acids, alpha-hydroxy acids (such as citric acid, lactic acid, malic acid) and their derivatives, mono-, di- and oligosaccharides such as glucose, galactose, fructose, mannose, fructose and lactose, polysugars such as R-glucans, in particular 1,3-1,4-β-glucan from oats, alpha-hydroxy fatty acids, triterpene acids such as betulinic acid or ursolic acid, and algae extracts.

Depending on the substance, the concentration of the moisture retention regulators used is between 0.1 and 10% (m/m) and preferably between 0.5 and 5% (m/m), based on the total weight of a ready-to-use cosmetic or pharmaceutical end product. These data apply in particular to such diols as are advantageously to be used, such as hexylene glycol, 1,2-pentanediol, 1,2-hexanediol, 1,2-octanediol and 1,2-decanediol, as well as mixtures of 1,2-hexanediol and 1,2-octanediol.

The use of cooling agents in cosmetic and pharmaceutical preparations can alleviate itching. The composition or cosmetic and/or pharmaceutical preparation according to the present invention can therefore also be particularly advantageously combined with one or more cooling agent(s). Preferred individual cooling agents for use within the framework of the present invention are listed below. The person skilled in the art can add many other cooling agents to this list; the cooling agents listed can also be used in combination with one another: I-menthol, d-menthol, racemic menthol, menthone glycerol acetal (trade name: Frescolat® MGA), menthyl lactate (trade name: Frescolat® ML; menthyl lactate is preferably l-menthyl lactate, in particular l-menthyl l-lactate), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate, N-acetylglycine menthyl ester, isopulegol, hydroxycarboxylic acid menthyl esters (such as menthyl 3-hydroxybutyrate), monomenthyl succinate, 2-mercaptocyclodecanone, menthyl 2-pyrrolidin-5-one carboxylate, 2,3-dihydroxy-p-menthane, 3,3,5-trimethyl cyclohexanone glycerol ketal, 3-menthyl-3,6-di- and trioxaalkanoates, 3-menthyl methoxyacetate and icilin.

Cooling agents which are preferred due to their particular synergistic effect are l-menthol, d-menthol, racemic menthol, menthone glycerol acetal (trade name: Frescolat® MGA), menthyl lactate (preferably l-menthyl lactate, in particular l-menthyl l-lactate (trade name: Frescolat® ML)), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate and isopulegol.

Particularly preferred cooling agents are l-menthol, racemic menthol, menthone glycerol acetal (trade name: Frescolat® MGA), menthyl lactate (preferably l-menthyl lactate, in particular l-menthyl l-lactate (trade name: Frescolat® ML)), 3-menthoxypropane-1,2-diol, 2-hydroxyethyl menthyl carbonate and 2-hydroxypropyl menthyl carbonate.

Very particularly preferred cooling agents are l-menthol, menthone glycerol acetal (trade name: Frescolat® MGA) and menthyl lactate (preferably l-menthyl lactate, in particular l-menthyl l-lactate (trade name: Frescolat® ML)).

Depending on the substance, the concentration of the cooling agents used is preferably between 0.01 and 20 wt % and particularly preferably between 0.1 and 5 wt %, based on the total weight of a ready-to-use cosmetic or pharmaceutical end product.

The composition or cosmetic and/or pharmaceutical preparation according to the present invention can also be used together with one or more osmolyte(s). Examples of osmolytes which may be mentioned here include substances from the group comprising sugar alcohols (myoinositol, mannitol, sorbitol), quaternary amines such as taurine, choline, betaine, betaine glycine, ectoin, diglycerol phosphate, phosphorylcholine or glycerophosphorylcholines, amino acids such as glutamine, glycine, alanine, glutamate, aspartate or proline, phosphatidylcholine, phosphatidylinositol, inorganic phosphates, and polymers of said compounds, such as proteins, peptides, polyamino acids and polyols. All osmolytes simultaneously have a skin-moisturising action.

Preferably, keratolytic substances can also be combined with the formulation according to the present invention. Keratolytic compounds include the large group of alpha-hydroxy acids. Salicylic acid is for example preferably used.

In cosmetic and/or pharmaceutical preparations containing the composition according to the present invention for the topical cosmetic or pharmaceutical treatment of for example dry and/or itchy skin, a high proportion of in particular nurturing substances is also particularly advantageous because of the reduced trans-epidermal water loss due to lipophilic components. In one preferred embodiment, the cosmetic and/or pharmaceutical preparation contains one or more nurturing animal and/or vegetable fats and oils such as olive oil, sunflower oil, refined soybean oil, palm oil, sesame oil, rapeseed oil, almond oil, borage oil, evening primrose oil, coconut oil, shea butter, jojoba oil, sperm oil, tallow, neatsfoot oil and lard, and optionally other nurturing components such as fatty alcohols having 8 to 30 C atoms. The fatty alcohols used here can be either saturated or unsaturated and either linear or branched. Nurturing substances which can be particularly preferably combined with the mixtures according to the present invention also include in particular ceramides, understood here to mean N-acylsphingosines (fatty acid amides of sphingosine) or synthetic analogues of such lipids (so-called pseudo-ceramides) which markedly improve the water retention capacity of the stratum corneum; phospholipids, such as soy lecithin, egg lecithin and cephalins; and petrolatum, paraffin oils and silicone oils, the latter including inter alia dialkyl- and alkylarylsiloxanes such as dimethylpolysiloxane and methylphenylpolysiloxane and their alkoxylated and quaternised derivatives.

The composition or cosmetic and/or pharmaceutical preparation according to the present invention can also contain one or more anti-inflammatory substance(s) and/or substances that alleviate reddening and/or other substances that alleviate itching, which in this context includes all anti-inflammatory active substances and active substances that alleviate reddening and itching and are suitable and/or conventionally used for cosmetic and/or dermatological applications. Steroidal anti-inflammatory substances of the corticosteroid type, such as hydrocortisone, hydrocortisone derivatives such as hydrocortisone 17-butyrate, dexamethasone, dexamethasone phosphate, methylprednisolone or cortisone, are advantageously used as anti-inflammatory compounds or compounds that alleviate reddening and/or itching; other steroidal anti-inflammatories can also be added to this list. It is also possible to use non-steroidal anti-inflammatories; examples which may be mentioned here include oxicams such as piroxicam or tenoxicam; salicylates such as aspirin, Disalcid®, Solprin® or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen or benoxaprofen; or pyrazoles such as phenylbutazone, oxyphenylbutazone, febrazone or azapropazone. A possible alternative is to use natural anti-inflammatory substances or substances that alleviate reddening and/or itching. Plant extracts, special high-activity plant extract fractions and high-purity active substances isolated from plant extracts can be used. Particular preference is afforded to extracts, fractions and active substances from camomile, Aloe vera, Commiphora species, Rubia species, willow, willow-herb, oats, calendula, arnica, St John's wort, honeysuckle, rosemary, Passiflora incarnata, witch hazel, ginger or Echinacea, and pure substances such as inter alia (alpha-)bisabolol, apigenin, apigenin-7-glucoside, boswellic acid, phytosterols, glycyrrhizin, glabridin, gingerols such as [6]-gingerol, paradols such as [6]-paradol and licochalcone A. These formulations can also contain mixtures of two or more anti-inflammatory active compounds.

Depending on the substance, the concentration of the anti-inflammatory compounds which can be used is between 0.005 and 2% (m/m) and preferably between 0.05 and 0.5% (m/m), based on the total weight of a ready-to-use cosmetic or pharmaceutical end product. These data apply in particular to bisabolol.

Other antibacterial or antimycotic active substances can also particularly advantageously be used in the composition or cosmetic and/or pharmaceutical preparation according to the present invention, wherein any antibacterial or antimycotic active substances can be used which are suitable or customary in cosmetic and/or pharmaceutical applications. In addition to the large group of conventional antibiotics, other products which are advantageous here include for example in particular triclosan, climbazole, octoxyglycerin, Octopirox® (1-hydroxy-4-methyl-6-(2,4,4-trimethylpentyl)-2(1H)-pyridone 2-aminoethanol salt), chitosan, farnesol, glycerol monolaurate or combinations of said substances, which are used inter alia against underarm odour, foot odour or dandruff.

The composition or cosmetic and/or pharmaceutical preparation according to the present invention can also contain one or more lenitive substance(s), wherein any lenitive substances can be used which are suitable or customary in cosmetic and/or pharmaceutical applications, such as alpha-bisabolol, azulene, guaiazulene, 18-beta-glycyrrhetinic acid, Laureth-9, Trideceth-9, 4-t-Butylcyclohexanol.

The composition or cosmetic and/or pharmaceutical preparation according to the pressure invention can also be combined with one or more cosmetically or pharmaceutically acceptable excipients such as those conventionally used in such preparations, for example antioxidants, preservatives, (metal) chelating agents, surface-active substances, emulsifiers, perfume oils, anti-foaming agents, colorants, pigments having a colouring action, thickeners, plasticisers, fats, oils, waxes or other conventional components of a cosmetic formulation, such as alcohols, polyols, polymers, foam stabilisers, electrolytes, organic solvents or silicone derivatives. Any conceivable antioxidants, preservatives, (metal) chelating agents, surface-active substances, emulsifiers, perfume oils, anti-foaming agents, colorants, pigments having a colouring action, thickeners, plasticisers, fats, oils, waxes or other conventional components of a cosmetic or pharmaceutical formulation, such as alcohols, polyols, polymers, foam stabilisers, electrolytes, organic solvents or silicone derivatives that are suitable or conventionally used for cosmetic and/or pharmaceutical applications can be used here in accordance with the invention.

The composition or cosmetic and/or pharmaceutical preparation according to the invention can also particularly advantageously contain one or more antioxidant(s), wherein any antioxidants can be used which are suitable or conventionally used for cosmetic and/or pharmaceutical applications. Advantageously, the antioxidants are selected from the group consisting of amino acids (for example glycine, histidine, tyrosine, tryptophan) and their derivatives, imidazoles (for example urocanic acid) and their derivatives, peptides such as D,L-carnosine, D-carnosine, L-carnosine and their derivatives (for example anserine), carotenoids, carotenes (for example α-carotene, β-carotene, lycopene) and their derivatives, lipoic acid and its derivatives (for example dihydrolipoic acid), aurothioglucose, propylthiouracil and other thiols (for example thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and their derivatives (esters, ethers, peptides, lipids, nucleotides, nucleosides and salts) as well as sulphoximine compounds (for example buthionine sulphoximines, homocysteine sulphoximines, buthionine sulphones, penta-, hexa-, hepta-thionine sulphoximine) in very low tolerated doses, and also (metal) chelating agents, for example α-hydroxy fatty acids, palmitic acid, phytic acid, lactoferrin, α-hydroxy acids (for example citric acid, lactic acid, malic acid), humic acid, bile acid, bile extracts, bilirubin, biliverdin, EDTA, EGTA and their derivatives, unsaturated fatty acids and their derivatives (for example γ-linolenic acid, linoleic acid, oleic acid), folic acid and its derivatives, ubiquinone and ubiquinol and their derivatives, Vitamin C and its derivatives (for example ascorbyl palmitate, magnesium ascorbyl phosphate, ascorbyl acetate), tocopherols and their derivatives (for example Vitamin E acetate), Vitamin A and its derivatives (for example Vitamin A palmitate) and also coniferyl benzoate of benzoin resin, rutinic acid and its derivatives, ferrulic acid and its derivatives, butylhydroxytoluene, butylhydroxyanisole, nordihydroguaiacic acid, nordihydroguaiaretic acid, trihydroxybutyrophenone, uric acid and its derivatives, mannose and its derivatives, zinc and its derivatives (for example ZnO, ZnSO₄), selenium and its derivatives (such as selenium methionine), stilbenes and their derivatives (such as stilbene oxide, trans-stilbene oxide), as well as the derivatives (such as salts, esters, ethers, sugars, nucleotides, nucleosides, peptides and lipids) of said active compounds such as are suitable in accordance with the invention.

The composition or cosmetic and/or pharmaceutical preparation according to the present invention can also particularly advantageously contain one or more substance(s) for preservative purposes, wherein any preservatives may be used which are suitable or customary in cosmetic and/or pharmaceutical applications and which are advantageously selected from the group consisting of preservatives such as inter alia benzoic acid, its esters and salts; propionic acid and its salts; salicylic acid and its salts; 2,4-hexanoic acid (sorbic acid) and its salts; formaldehyde and paraformaldehyde; 2-hydroxybiphenyl ether and its salts; 2-zincsulphidopyridine N-oxide; inorganic sulphites and bisulphites; sodium iodate; chlorobutanol; 4-hydroxybenzoic acid and its salts and esters; dehydroacetic acid; formic acid; 1,6-bis(4-amidino-2-bromophenoxy)-n-hexane and its salts; the sodium salt of ethylmercury-(II)-thiosalicylic acid; phenylmercury and its salts; 10-undecylenic acid and its salts; 5-amino-1,3-bis(2-ethylhexyl)-5-methylhexahydropyrimidine; 5-bromo-5-nitro-1,3-dioxane; 2-bromo-2-nitro-1,3-propanediol; 2,4-dichlorobenzyl alcohol; N-(4-chlorophenyl)-N′-(3,4-dichlorophenyl)urea; 4-chloro-m-cresol; 2,4,4′-trichloro-2′-hydroxy-diphenyl ether; 4-chloro-3,5-dimethylphenol; 1,1′-methylene-bis(3-(1-hydroxymethyl-2,4-dioximidazolidin-5-yl)urea); poly(hexamethylene biguanide) hydrochloride; 2-phenoxyethanol; hexamethylenetetramine; 1-(3-chloroallyl)-3,5,7-triaza-1-azoniaadamantane chloride; 1-(4-chloro-phenoxy)-1(1H-imidazol-1-yl)-3,3-dimethyl-2-butanone; 1,3-bis(hydroxymethyl)-5,5-dimethyl-2,4-imidazolidinedione; benzyl alcohol; Octopirox®; 1,2-dibromo-2,4-dicyanobutane; 2,2′-methylene-bis(6-bromo-4-chloro-phenol); bromochlorophene; mixture of 5-chloro-2-methyl-3(2H)-isothiazolinone and 2-methyl-3(2H)isothiazolinone with magnesium chloride and magnesium nitrate; 2-benzyl-4-chlorophenol; 2-chloroacetamide; chlorhexidine; chlorhexidine acetate; chlorhexidine gluconate; chlorhexidine hydrochloride; 1-phenoxy-propan-2-ol; N-alkyl(C12-C22)trimethylammonium bromide and chloride; 4,4-dimethyl-1,3-oxazolidine; N-hydroxymethyl-N-(1,3-di(hydroxymethyl)-2,5-dioxoimidazolidin-4-yl)-N′-hydroxymethylurea; 1,6-bis(4-amidinophenoxy)-n-hexane and its salts; glutaraldehyde 5-ethyl-1-aza-3,7-dioxabicyclo(3.3.0)octane; 3-(4-chlorophenoxy)-1,2-propanediol; hyamine; alkyl(C8-C18)dimethylbenzylammonium chloride; alkyl(C8-C18)dimethylbenzylammonium bromide; alkyl(C8-C18)dimethylbenzylammonium saccharinate; benzylhemiformal; 3-iodo-2-propynyl butylcarbamate; or sodium ((hydroxymethyl)amino)acetate.

The composition or cosmetic and/or pharmaceutical preparation according to the present invention can also particularly advantageously contain one or more (metal) chelating agent(s), wherein any metal chelating agents can be used which are suitable or customary in cosmetic and/or pharmaceutical applications. Preferred (metal) chelating agents include α-hydroxy fatty acids, phytic acid, lactoferrin, α-hydroxy acids, such as inter alia citric acid, lactic acid and malic acid, as well as humic acids, bile acids, bile extracts, bilirubin, biliverdin or EDTA, EGTA and their derivatives.

The composition or cosmetic and/or pharmaceutical preparation can also particularly advantageously contain one or more anionic, cationic, non-ionic and/or amphoteric surfactant(s), in particular if crystalline or microcrystalline solids, for example inorganic micropigments, are to be incorporated into the preparations. Surfactants are amphiphilic substances capable of solubilising organic, non-polar substances in water. The hydrophilic parts of a surfactant molecule are usually polar functional groups, such as —COO⁻, —OSO₃⁻ or —SO₃⁻, while the hydrophobic parts are normally non-polar hydrocarbon radicals. Surfactants are generally classified according to the type and charge of the hydrophilic part of the molecule. They can be divided into four groups: anionic surfactants, cationic surfactants; amphoteric surfactants; and non-ionic surfactants.

Anionic surfactants normally contain carboxylate, sulphate or sulphonate groups as functional groups. In aqueous solution, they form negatively charged organic ions in an acidic or neutral medium. Cationic surfactants are characterised virtually exclusively by the presence of a quaternary ammonium group. In aqueous solution they form positively charged organic ions in an acidic or neutral medium. Amphoteric surfactants contain both anionic and cationic groups and accordingly behave like anionic or cationic surfactants in aqueous solution, depending on the pH value. They have a positive charge in a strongly acidic medium and a negative charge in an alkaline medium. In the neutral pH range, by contrast, they are zwitterionic. Polyether chains are typical of non-ionic surfactants. Non-ionic surfactants do not form ions in an aqueous medium.

Anionic surfactants that can advantageously be used include: acyl amino acids (and their salts), such as acyl glutamates, for example sodium acyl glutamate, di-TEA-palmitoyl aspartate and sodium caprylic/capric glutamate; acyl peptides, for example palmitoyl-hydrolysed lactoprotein, sodium cocoyl-hydrolysed soy protein and sodium/potassium cocoyl-hydrolysed collagen; sarcosinates, for example myristoyl sarcosinate, TEA-lauroyl sarcosinate, sodium lauroyl sarcosinate and sodium cocoyl sarcosinate; taurates, for example sodium lauroyl taurate and sodium methyl cocoyl taurate; acyl lactylates, for example lauroyl lactylate and caproyl lactylate; alaninates; carboxylic acids and derivatives, such as for example lauric acid, aluminium stearate, magnesium alkanolate and zinc undecylenate; ester carboxylic acids, for example calcium stearoyl lactylate, laureth-6 citrate and sodium PEG-4 lauramide carboxylate; ether carboxylic acids, for example sodium laureth-13 carboxylate and sodium PEG-6 cocamide carboxylate; phosphoric acid esters and salts, such as for example DEA-oleth-10 phosphate and dilaureth-4 phosphate; sulphonic acids and salts, such as acyl isethionates, for example sodium/ammonium cocoyl isethionate; alkyl aryl sulphonates; alkyl sulphonates, for example sodium cocomonoglyceride sulphonate, sodium C12-14 olefin sulphonate, sodium lauryl sulphoacetate and magnesium PEG-3 cocamide sulphate; sulphosuccinates, for example dioctyl sodium sulphosuccinate, disodium laureth sulphosuccinate, disodium lauryl sulphosuccinate and disodium undecylenamido MEA-sulphosuccinate; and sulphuric acid esters, such as alkyl ether sulphate, for example sodium, ammonium, magnesium, MIPA, TIPA laureth sulphate, sodium myreth sulphate and sodium C12-13 pareth sulphate, and alkyl sulphates, for example sodium, ammonium and TEA lauryl sulphate.

Cationic surfactants that can advantageously be used include alkyl amines, alkyl imidazoles, ethoxylated amines and quaternary surfactants:

RNH₂CH₂CH₂COO⁻ (at pH 7);
RNHCH₂CH₂COO⁻B⁺ (at pH 12), where B⁺ is an arbitrary cation such as Na⁺;
esterquats.

Quaternary surfactants contain at least one N atom that is covalently bonded to four alkyl or aryl groups. This leads to a positive charge, irrespective of the pH value. Alkyl betaine, alkyl amidopropyl betaine and alkyl amidopropyl hydroxysulphaine are advantageous. The cationic surfactants used can also preferably be chosen from the group of quaternary ammonium compounds, in particular benzyl trialkyl ammonium chlorides or bromides, such as for example benzyl dimethylstearyl ammonium chloride, as well as alkyl trialkyl ammonium salts, for example cetyl trimethyl ammonium chloride or bromide, alkyl dimethyl hydroxyethyl ammonium chlorides or bromides, dialkyl dimethyl ammonium chlorides or bromides, alkyl amide ethyl trimethyl ammonium ether sulphates, alkyl pyridinium salts, for example lauryl or cetyl pyridinium chloride, imidazoline derivatives and compounds of a cationic nature, such as amine oxides, for example alkyl dimethyl amine oxides or alkyl aminoethyl dimethyl amine oxides. Cetyl trimethyl ammonium salts can particularly advantageously be used.

Amphoteric surfactants that can advantageously be used include: acyl/dialkyl ethylene diamine, for example sodium acyl amphoacetate, disodium acyl amphodipropionate, disodium alkyl amphodiacetate, sodium acyl amphohydroxypropyl sulphonate, disodium acyl amphodiacetate and sodium acyl amphopropionate; N-alkyl amino acids, for example aminopropyl alkyl glutamide, alkyl aminopropionic acid, sodium alkyl imidodipropionate and lauroamphocarboxyglycinate.

Non-ionic surfactants that can advantageously be used include: alcohols; alkanolamides, such as cocamides MEA/DEA/MIPA, amine oxides, such as cocoamidopropylamine oxide; esters formed by esterification of carboxylic acids with ethylene oxide, glycerol, sorbitan or other alcohols; ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated polyoxyethylene (POE) ethers and alkyl polyglycosides, such as lauryl glucoside, decyl glycoside and cocoglycoside; sucrose esters and ethers; polyglycerol esters, diglycerol esters, monoglycerol esters; methyl glucose esters, esters of hydroxy acids.

The use of a combination of anionic and/or amphoteric surfactants with one or more non-ionic surfactants is also advantageous.

The surface-active substance can be present at a concentration of between 1 and 98% (m/m) in the composition or cosmetic and/or pharmaceutical preparation according to the present invention, based on the total weight of the formulations.

The composition or cosmetic and/or pharmaceutical preparation can also particularly advantageously contain one or more emulsifier(s) commonly used in the art for preparing cosmetic or pharmaceutical formulations. Oil-in-water (O/W) emulsifiers can for example be advantageously selected from the group comprising polyethoxylated or polypropoxylated or polyethoxylated and polypropoxylated products, such as fatty alcohol ethoxylates, ethoxylated wool wax alcohols, polyethylene glycol ethers of the general formula R—O—(—CH₂—CH₂—O—)ₙ—R′, fatty acid ethoxylates of the general formula R—COO—(—CH₂—CH₂—O—)ₙ—H, etherified fatty acid ethoxylates of the general formula R—COO—(—CH₂—CH₂—O—)ₙ—R′, esterified fatty acid ethoxylates of the general formula R—COO—(—CH₂—CH₂—O—)ₙ—C(O)—R′, polyethylene glycol glycerol fatty acid esters, ethoxylated sorbitan esters, cholesterol ethoxylates, ethoxylated triglycerides, alkyl ether carboxylic acids of the general formula R—COO—(—CH₂—CH₂—O—)ₙ—OOH, where n is a number from 5 to 30, polyoxyethylene sorbitol fatty acid esters, alkyl ether sulphates of the general formula R—O—(—CH₂—CH₂—O—)ₙ—SO₃—H, fatty alcohol propoxylates of the general formula R—O—(—CH₂—CH(CH₃)—O—)ₙ—H, polypropylene glycol ethers of the general formula R—O—(—CH₂—CH(CH₃)—O—)ₙ—R, propoxylated wool wax alcohols, etherified fatty acid propoxylates R—COO—(—CH₂—CH(CH₃)—O—)ₙ—R, esterified fatty acid propoxylates of the general formula R—COO—(—CH₂—CH(CH₃)—O—)ₙ—C(O)—R′, fatty acid propoxylates of the general formula R—COO—(—CH₂—CH(CH₃)—O—)ₙ—H, polypropylene glycol glycerol fatty acid esters, propoxylated sorbitan esters, cholesterol propoxylates, propoxylated triglycerides, alkyl ether carboxylic acids of the general formula R—O— (—CH₂—CH(CH₃)—O—)ₙ—CH₂—COOH, alkyl ether sulphates (and the acids on which these sulphates are based) of the general formula R—O—(—CH₂—CH(CH₃)—O—)ₙ—SO₃—H, fatty alcohol ethoxylates/propoxylates of the general formula R—O—Xₙ—Yₘ—H, polypropylene glycol ethers of the general formula R—O—Xₙ—Yₙ—R′, etherified fatty acid propoxylates of the general formula R—COO—Xₙ—Yₙ—R′, and fatty acid ethoxylates/propoxylates of the general formula R—COO—Xₙ—Yₘ—H.

In accordance with the invention, the polyethoxylated or polypropoxylated or polyethoxylated and polypropoxylated O/W emulsifiers used are particularly advantageously selected from the group comprising substances having HLB values of 11 to 18, more particularly advantageously 14.5 to 15.5, if the O/W emulsifiers contain saturated radicals R and R′. If the O/W emulsifiers contain unsaturated radicals R and/or R′, or if isoalkyl derivatives are present, then the preferred HLB value of such emulsifiers can also be lower or higher. The fatty alcohol ethoxylates are advantageously selected from the group comprising ethoxylated stearyl alcohols, cetyl alcohols and cetylstearyl alcohols (cetearyl alcohols).

The following emulsifiers are particularly preferred: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19) and polyethylene glycol (20) cetylstearyl ether (ceteareth-20).

The fatty acid ethoxylates are also advantageously selected from the following group: polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate and polyethylene glycol (20) oleate.

Sodium laureth-11 carboxylate can advantageously be used as an ethoxylated alkyl ether carboxylic acid or its salt. Sodium laureth-14 sulphate can advantageously be used as an alkyl ether sulphate. Polyethylene glycol (30) cholesteryl ether can advantageously be used as an ethoxylated cholesterol derivative. Polyethylene glycol (25) soy sterol has also proven useful.

Polyethylene glycol (60) evening primrose glycerides can advantageously be used as ethoxylated triglycerides.

The polyethylene glycol glycerol fatty acid esters are also advantageously selected from the group comprising polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprylate/caprate, polyethylene glycol (20) glyceryl oleate, polyethylene glycol (20) glyceryl isostearate and polyethylene glycol (18) glyceryl oleate/cocoate.

The sorbitan esters are likewise favourably selected from the group comprising polyethylene glycol (20) sorbitan monolaurate, polyethylene glycol (20) sorbitan monostearate, polyethylene glycol (20) sorbitan monoisostearate, polyethylene glycol (20) sorbitan monopalmitate and polyethylene glycol (20) sorbitan monooleate.

The following can be used as advantageous W/O emulsifiers: fatty alcohols having 8 to 30 carbon atoms; monoglycerol esters of saturated and/or unsaturated, branched and/or unbranched alkane carboxylic acids having a chain length of 8 to 24, in particular 12 to 18 C atoms; diglycerol esters of saturated and/or unsaturated, branched and/or unbranched alkane carboxylic acids having a chain length of 8 to 24, in particular 12 to 18 C atoms; monoglycerol ethers of saturated and/or unsaturated, branched and/or unbranched alcohols having a chain length of 8 to 24, in particular 12 to 18 C atoms; diglycerol ethers of saturated and/or unsaturated, branched and/or unbranched alcohols having a chain length of 8 to 24, in particular 12 to 18 C atoms; propylene glycol esters of saturated and/or unsaturated, branched and/or unbranched alkane carboxylic acids having a chain length of 8 to 24, in particular 12 to 18 C atoms; and sorbitan esters of saturated and/or unsaturated, branched and/or unbranched alkane carboxylic acids having a chain length of 8 to 24, in particular 12 to 18 C atoms.

Particularly advantageous W/O emulsifiers include: glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprate and glyceryl monocaprylate.

The composition according to the present invention can also be used as a component of perfume compositions for haircare and scalpcare products and, in particular because of their specific efficacy, can impart an additional itch-alleviating or antiallergic property to for example a perfumed finished product. Particularly preferred perfume compositions comprise (a) a sensorially effective amount of a perfume, (b) an itch-regulating, antiallergic and/or hyposensitising amount of a synergistically effective mixture of anthranilic acid amides and antidandruff agents, and (c) optionally, one or more excipients and/or additives. It has proven particularly advantageous that the composition comprising at least one avenanthramide oran analogue thereof, or a preparation comprising at least one avenanthramide or an analogue thereof have only a weak inherent odour or are even completely odourless, since this property lends them to use in a perfume composition in particular.

With regard to other cosmetically and pharmaceutically acceptable excipients, bases and auxiliaries which can particularly preferably be combined with the formulation according to the invention, reference may be made to the detailed descriptions in WO 2007/062957 and WO 2003/069994, the relevant disclosure of which is hereby incorporated by reference.

The composition according to the present invention for delivering the active substance(s), i. e. avenanthramide(s), can be incorporated without difficulty into conventional cosmetic or dermatological or keratological preparations such as inter alia pump sprays, aerosol sprays, creams, shampoos, ointments, tinctures, lotions, nailcare products (such as nail varnishes, nail varnish removers, nail balsams) and the like. Within this context, the cosmetic and/or dermatological or keratological compositions comprising at least one avenanthramide or an analogue thereof, or a preparation comprising at least one avenanthramide or an analogue thereof can otherwise be conventional in composition and can be used for treating the skin, hair and/or nails within the context of cosmetic care or a dermatological or keratological treatment.

If the cosmetic or pharmaceutical preparation is a solution or lotion, then solvents which can be used include: water or aqueous solutions; fatty oils, fats, waxes and other natural and synthetic fatty bodies, preferably esters of fatty acids with alcohols having a low C number, such as isopropanol, propylene glycol or glycerol, or esters of fatty alcohols with alkanoic acids having a low C number or with fatty acids; alcohols, diols or polyols having a low C number, and their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products. Mixtures of the aforementioned solvents are in particular used. In the case of alcoholic solvents, water can be an additional constituent.

The cosmetic or pharmaceutical formulation can also be formulated in a form suitable for topical application, for example as lotions, aqueous or aqueous-alcoholic gels, vesicle dispersions or as simple or complex emulsions (O/W, W/O, O/W/O or W/O/W), liquids, semi-liquids or solids, such as milks, creams, gels, cream-gels, pastes or sticks, and can optionally be packaged as an aerosol and take the form of mousses or sprays. Such formulations are prepared according to usual methods.

For preparing emulsions, the oil phase can advantageously be chosen from the following group of substances: mineral oils, mineral waxes; fatty oils, fats, waxes and other natural and synthetic fatty bodies, preferably esters of fatty acids with alcohols having a low C number, for example with isopropanol, propylene glycol or glycerol, or esters of fatty alcohols with alkanoic acids having a low C number or with fatty acids; alkyl benzoates; silicone oils such as dimethyl polysiloxanes, diethyl polysiloxanes, diphenyl polysiloxanes and mixed forms thereof.

Advantageously, esters of saturated and/or unsaturated, branched and/or straight-chain alkane carboxylic acids having a chain length of 3 to 30 C atoms and saturated and/or unsaturated, branched and/or straight-chain alcohols having a chain length of 3 to 30 C atoms, from the group of esters of aromatic carboxylic acids and saturated and/or unsaturated, branched and/or straight-chain alcohols having a chain length of 3 to 30 C atoms can be used. Preferred ester oils include isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters, for example jojoba oil.

In addition, the oily phase can advantageously be selected from the group comprising branched and unbranched hydrocarbons and waxes, silicone oils, dialkyl ethers, the group comprising saturated or unsaturated, branched or unbranched alcohols, and also fatty acid triglycerides, specifically the triglycerol esters of saturated and/or unsaturated, branched and/or unbranched alkane carboxylic acids having a chain length of 8 to 24 and in particular 12 to 18 C atoms. The fatty acid triglycerides can advantageously be selected from the group comprising synthetic, semi-synthetic and natural oils, such as olive oil, sunflower oil, soybean oil, peanut oil, rapeseed oil, almond oil, palm oil, coconut oil, palm kernel oil and the like. Arbitrary mixtures of such oil and wax components can also advantageously be used. In some cases, it is also advantageous to use waxes, such as cetyl palmitate, as the sole lipid component of the oily phase; advantageously, the oily phase is selected from the group comprising 2-ethylhexyl isostearate, octyldodecanol, isotridecyl isononanoate, isoeicosane, 2-ethylhexyl cocoate, C12-15 alkyl benzoate, caprylic/capric triglyceride and dicaprylyl ether. Mixtures of C12-15 alkyl benzoate and 2-ethylhexyl isostearate, mixtures of C12-15 alkyl benzoate and isotridecyl isononanoate and mixtures of C12-15 alkyl benzoate, 2-ethylhexyl isostearate and isotridecyl isononanoate are particularly advantageous. The hydrocarbons paraffin oil, squalane and squalene can also advantageously be used. The oily phase can advantageously also contain cyclic or linear silicone oils or consist entirely of such oils, although other oily phase components are preferably used in addition to the silicone oil(s). Cyclomethicone (for example, decamethylcyclopentasiloxane) can advantageously be used as a silicone oil. However, other silicone oils can also advantageously be used, including for example undecamethylcyclotrisiloxane, polydimethylsiloxane and poly(methylphenylsiloxane). Mixtures of cyclomethicone and isotridecyl isononanoate and of cyclomethicone and 2-ethylhexyl isostearate are also particularly advantageous.

The aqueous phase of compositions or cosmetic of pharmaceutical preparations accoding to the present invention and taking the form of an emulsion can advantageously comprise alcohols, diols or polyols having a low C number, as well as their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols having a low C number, such as ethanol, isopropanol, 1,2-propanediol and glycerol, and in particular one or more thickeners, which can advantageously be selected from the group comprising silicon dioxide, aluminium silicates, polysaccharides and their derivatives, such as hyaluronic acid, xanthan gum, hydroxypropyl methyl cellulose, and particularly advantageously from the group comprising polyacrylates, preferably a polyacrylate from the group comprising so-called carbopols, such as type 980, 981, 1382, 2984 and 5984 carbopols, each on their own or in combinations.

A high content of treatment substances is usually advantageous in cosmetic or pharmaceutical preparations for the topical treatment of the skin. In accordance with a preferred variant, the preparation contains one or more animal and/or vegetable treatment fats and oils, such as olive oil, sunflower oil, purified soybean oil, palm oil, sesame oil, rapeseed oil, almond oil, borage oil, evening primrose oil, coconut oil, shea butter, jojoba oil, sperm oil, beef tallow, neatsfoot oil and lard, and optionally other treatment constituents such as for example C8-C30 fatty alcohols. The fatty alcohols used here can be saturated or unsaturated and straight-chain or branched, wherein examples include decanol, decenol, octanol, octenol, dodecanol, dodecenol, octadienol, decadienol, dodecadienol, oleyl alcohol, ricinoleyl alcohol, erucic alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well their guerbet alcohols; this list may be extended as desired to include other alcohols which structurally are chemically related. The fatty alcohols preferably originate from natural fatty acids and are usually prepared from the corresponding esters of the fatty acids by reduction. Fatty alcohol fractions formed by reduction from naturally occurring fats and fat oils can also be used, such as for example beef tallow, peanut oil, colza oil, cottonseed oil, soybean oil, sunflower oil, palm kernel oil, linseed oil, maize oil, castor oil, rapeseed oil, sesame oil, cocoa butter and cocoa fat.

The treatment substances that can preferably be combined with the cosmetic or pharmaceutical preparation according to the present invention can also include: ceramides, being understood to be N-acylsphingosines (fatty acid amides of sphingosine) or synthetic analogues of such lipids (so-called pseudo-ceramides) which clearly improve the water retention capacity of the stratum corneum; phospholipids, for example soy lecithin, egg lecithin and cephalins; Vaseline, paraffin and silicone oils, the latter including inter alia dialkyl- and alkylaryl-siloxanes such as dimethylpolysiloxane and methylphenylpolysiloxane, as well as their alkoxylated and quaternised derivatives.

Hydrolysed animal and/or vegetable proteins can also advantageously be added to the cosmetic or pharmaceutical preparation containing the composition according to the present invention. Advantageous examples in this regard include in particular elastin, collagen, keratin, lactoprotein, soy protein, oat protein, pea protein, almond protein and wheat protein fractions or corresponding hydrolysed proteins, as well as their condensation products with fatty acids, and also quaternised hydrolysed proteins, wherein the use of hydrolysed vegetable proteins is preferred.

The cosmetic or pharmaceutical preparation may also include a cosmetically or pharmaceutically acceptable carrier, such as (without being limited to) one of the following which are commonly used in the art: lactose, dextrose, sucrose, sorbitol, mannitol, starch, acacia rubber, calcium phosphate, alginate, gelatine, calcium silicate, microcrystalline cellulose, polyvinyl pyrrolidone, cellulose, water, syrup, methyl cellulose, methyl hydroxybenzoate, propyl hydroxybenzoate, talc, magnesium stearate, mineral oil and the like. The cosmetic or pharmaceutical formulations may also include lubricants, wetting agents, sweeteners, flavouring agents, emulsifiers, suspensions, preserving agents and the like, in addition to the above components. Suitable pharmaceutically acceptable carriers and formulations are described in detail in Remington's Pharmaceutical Sciences (19^{th} edition, 1995).

In order to be used, the composition or cosmetic or pharmaceutical, i. e. dermatological or keratological preparations are applied to the skin, scalp, hair and/or nails in an adequate amount and in such manner as is customary with cosmetics or pharmaceutical products.

Finally, the present invention relates to a method for preparing the composition according to the present invention, comprising the steps of:
providing at least one avenanthramide or an analogue thereof; and
adding and mixing at least one penetration enhancer to said avenanthramide or an analogue thereof.

The thus obtained composition can be added to the end, i.e. customary, preparation or formulation.

Alternatively, the individual constituents of the composition according to the present invention can be added separately to the end formulation.

While the invention has been specifically shown and described with reference to preferred variants, it will be understood by those skilled in the art that various changes in form and detail may be made to it without departing from the spirit and scope of the invention. Moreover, the invention encompasses any combination of the elements described above, in all possible variations, unless specifically indicated otherwise.

In addition, where features or aspects of the invention are described in terms of Markush groups, those skilled in the art will recognise that the invention is also thereby described in terms of any individual member or sub-group of members of the Markush group.

The present invention shall now be described in detail with reference to the following examples, which are merely illustrative of the present invention, such that the content of the present invention is not limited by or to the following examples.

### EXAMPLES

The avenanthramides (Avns) used in the examples were synthesised according to known processes, such as those detailed in WO 2004/047833.

For more accurate quantitative analysis only, the concentration of avenanthramide(s) used in the following examples is higher than the concentration of avenathramide(s) in the composition according to the present invention.

### Example 1: Investigation of the Permeation Behaviour of Avn A

The permeation of Avn A from a cosmetic formulation across a stratum corneum-mimicking artificial membrane was evaluated using the Skin PAMPA™ (Parallel Artificial Membrane Permeability Assay) model.

The primary penetration barrier of the skin, the stratum corneum (SC), consists of corneocytes embedded in extracellular lipids. The lipid mixture of the Skin PAMPA™ membrane consists of similar quantities of certramides (synthetic ceramide analogues), stearic acid and cholesterol as the SC. The Skin PAMPA™ sandwich consists of two 96-well plates, wherein one plate is formed so as to sit precisely under the plate that contains a porous lipid-impregnated filter. The wells of the bottom plate were filled with formulation, and the wells of the top plate were filled with acceptor solution. The plates were then stacked and incubated. The Skin PAMPA™ model has been used to evaluate semi-solid formulations and found to correlate well with ex vivo permeation studies. It has also been demonstrated that the Skin PAMPA™ system is favourably compatible with emulsions containing non-ionic surfactants.

TABLE 2
Formulations (amounts in % by weight)

| Raw material | INCI | A1 | A2 | A3 | A5 |
|---|---|---|---|---|---|
| Phase A | | | | | |
| Demineralised | Water (aqua) | ad 100 | | | |
| water | | | | | |
| EDTA BD | Disodium EDTA | 0.10 | 0.10 | 0.10 | 0.10 |
| Glycerine 99.5% | Glycerine | 0.50 | 0.50 | 0.50 | 0.50 |
| Phase B | | | | | |
| PCL Liquid 100 | Cetearyl ethyl hexanoate | 3.00 | 3.00 | 3.00 | 3.00 |
| Lanette O | Cetearyl alcohol | 2.00 | 2.00 | 2.00 | 2.00 |
| Neutral oil | Caprylic acid/capric | 3.00 | 3.00 | 3.00 | 3.00 |
| | triglycerides | | | | |
| Eutanol G | Octyl dodecanol | 4.00 | 4.00 | 4.00 | 4.00 |
| Phase C | | | | | |
| Pemulen ™ TR-1 | Acrylates/C10 - 30 alkyl | 0.20 | 0.20 | 0.20 | 0.20 |
| | acrylate cross-polymer | | | | |
| Phase D | | | | | |
| Sodium hydroxide | Sodium hydroxide | 0.40 | 0.40 | 0.40 | 0.40 |
| solution 10% | | | | | |
| Phase E | | | | | |
| Avn A | | 0.05 | 0.05 | 0.05 | 0.05 |
| Glycerine 99.5% | Glycerine | 1.00 | 1.00 | 1.00 | 1.00 |
| Hydrolite-5 | Pentylene glycol | — | 3.00 | 5.00 | — |
| Hydrolite-6 | 1,2-hexanediol | — | — | — | 2.00 |
| Demineralised water | Water (Aqua) | 2.00 | 2.00 | 2.00 | 2.00 |

| | |
|---|---|
| Sum | 100.00 |
| pH value | 5.5 |

Production Method:

Heat Phase A and B separately to 70° C.; disperse Phase C in B; add Phase BC to Phase A and emulsify using an ULTRA TURRAX® stirrer (2 minutes); allow to cool by using a vane stirrer; add Phase D for neutralising. The formulation was prepared in bulk. The active substances were pre-dissolved in the corresponding additives and were added to the formulation (15 minutes stirring with a vane stirrer).

Alternatively, it is also possible to presolve the corresponding avenanthramide in glycerine or ethanol or mixtures thereof and add the corresponding diol into the water phase of the emulsion. Both ways of incorporation deliver the same results.

Acceptor Solution:

180 ml of buffer was mixed with 20 ml ethanol (buffer: 20 mmol/l citric acid, pH 5.5);
0.84 g of anhydrous citric acid was dissolved in approximately 180 g of water;
the pH was adjusted to 5.5 using a 30% sodium hydroxide solution;
following pH adjustment, the solution was filled up with water to 200 g.

Sample Preparation:

After 240 minutes, approximately 180 μl of each Skin PAMPA™ sample was transferred into an HPLC micro-vial.

HPLC Analytical Conditions:

Equipment: an Agilent HP 1100 connected to a Waters Empower 3 SR3

### Column: a Waters XBridge® C18, 3.5 μm, 3.0×50 mm

Column temperature: 30° C.
Mobile phase A: water+0.1% formic acid
Mobile phase B: methanol
Flow: 1.02 ml/min
Injection volume: 25 μl

### Wavelength: 304 nm

| | Time [min] | % A | % B |
|---|---|---|---|
| Gradient Table: | Initial | 65.0 | 35.0 |
| | 6.00 | 50.0 | 50.0 |
| | 6.05 | 65.0 | 35.0 |
| | 8.00 | 65.0 | 35.0 |

The results are summarised in the following table.

TABLE 3
Formulations (amounts in % by weight)

| Content of | A1 | A2 | A3 | A5 |
|---|---|---|---|---|
| avenanthramide in | Placebo | 3% | 5% | 2% |
| acceptor solution | (no | Hydro- | Hydro- | Hydro- |
| (μg/ml) | additive) | lite-5 | lite-5 | lite-6 |
| 0 min | 0.000 | 0.000 | 0.000 | 0.000 |
| 240 min | 0.319 | 0.378 | 0.506 | 0.465 |
| % Modulation of | 0 | +19 | +59 | +46 |
| permeation vs A1 | | | | |

The results clearly show that adding 1,2-diols potently increases the permeation of Avn A. Modulation by Hydrolite-5 (1,2-pentanediol) was concentration-dependent, giving a 19% increase at a 3% dosage and a 59% increase at a 5% dosage. Hydrolite-6 (1,2-hexanediol) was even more efficient than Hydrolite-5, as it enhanced the permeation of Avn A by 46% at a 2% dosage, as compared to the 19% improvement from Hydrolite-5 at a 3% dosage.

1,2-Alkanediols possess skin-moisturising properties, thus providing additional benefits to skincare and scalpcare formations aimed at relieving itching, since itchy skin is often accompanied by dry, sensitive or damaged skin.

They also give the formulations a light and elegant feel on the skin, making them far more acceptable to users.

### Example 2: Investigation of the Permeation Behaviour of Avn L

The permeation of Avn L from a cosmetic formulation across a stratum corneum-mimicking artificial membrane was evaluated as described above using the Skin PAMPA™ (Parallel Artificial Membrane Permeability Assay) model.

TABLE 4
Formulations (% parts by weight)

| Raw material | INCI | L1 | L2 | L4 |
|---|---|---|---|---|
| Phase A | | | | |
| Demineralised water | Water (aqua) | ad 100 | | |
| EDTA BD | Disodium EDTA | 0.10 | 0.10 | 0.10 |
| Glycerine 99.5% | Glycerine | 0.50 | 0.50 | 0.50 |
| Phase B | | | | |
| PCL Liquid 100 | Cetearyl ethyl hexanoate | 3.00 | 3.00 | 3.00 |
| Lanette O | Cetearyl alcohol | 2.00 | 2.00 | 2.00 |
| Neutral oil | Caprylic acid/capric | 3.00 | 3.00 | 3.00 |
| | triglycerides | | | |
| Eutanol G | Octyl dodecanol | 4.00 | 4.00 | 4.00 |
| Phase C | | | | |
| Pemulen ™ TR-1 | Acrylates/C10 - 30 alkyl | 0.20 | 0.20 | 0.20 |
| | acrylate cross-polymer | | | |
| Phase D | | | | |
| Sodium hydroxide | Sodium hydroxide | 0.40 | 0.40 | 0.40 |
| solution 10% | | | | |
| Phase E | | | | |
| Avn L | | 0.05 | 0.05 | 0.05 |
| Glycerine 99.5% | Glycerine | 1.00 | 1.00 | 1.00 |
| Hydrolite-5 | Pentylene glycol | — | 3.00 | — |
| Hydrolite-6 | 1,2-hexanediol | — | — | 1.00 |
| Demineralised water | Water (Aqua) | 2.00 | 2.00 | 2.00 |

| | |
|---|---|
| Sum | 100.00 |
| pH value | 5.5 |

The results are summarised in the following table.

TABLE 5
Formulations (amounts in % by weight)

| Content of | L1 | | |
|---|---|---|---|
| avenanthramide in | Placebo | | |
| acceptor solution | (no | L2 | L4 |
| (μg/ml) | additive) | 3% Hydrolite-5 | 1% Hydrolite-6 |
| 0 min | 0.000 | 0.000 | 0.000 |
| 240 min | 0.030 | 0.068 | 0.078 |
| % Modulation of | 0 | +125 | +160 |
| permeation vs L1 | | | |

The results clearly show that adding 1,2-diols potently increases the permeation of Avn L. Modulation by Hydrolite-5 (1,2-pentanediol) at a 3% dosage gave a 125% increase. Hydrolite-6 (1,2-hexanediol) at a 1% dosage was even more efficient than Hydrolite-5 at a 3% dosage, as it enhanced the permeation of Avn L by 160%.

### Example 3: Investigation of the Permeation Behaviour of Avn B

The permeation of Avn B from a cosmetic formulation across a stratum corneum-mimicking artificial membrane was evaluated as described above using the Skin PAMPA™ (Parallel Artificial Membrane Permeability Assay) model.

Unlike Examples 1 and 2, acceptor solution samples for HPLC analysis of each Skin PAMPA™ sample were this time taken after just 120 minutes.

TABLE 6
Formulations (amounts in % by weight)

| Raw material | INCI | B1 | B3 | B4 | B5 | B6 |
|---|---|---|---|---|---|---|
| Phase A | | | | | | |
| Demineralised water | Water (aqua) | ad 100 | | | | |
| EDTA BD | Disodium EDTA | 0.10 | 0.10 | 0.10 | 0.10 | 0.10 |
| Glycerine 99.5% | Glycerine | 0.50 | 0.50 | 0.50 | 0.50 | 0.50 |
| Phase B | | | | | | |
| PCL Liquid 100 | Cetearyl ethyl hexanoate | 3.00 | 3.00 | 3.00 | 3.00 | 3.00 |
| Lanette O | Cetearyl alcohol | 2.00 | 2.00 | 2.00 | 2.00 | 2.00 |
| Neutral oil | Caprylic acid/capric | 3.00 | 3.00 | 3.00 | 3.00 | 3.00 |
| | triglycerides | | | | | |
| Eutanol G | Octyl dodecanol | 4.00 | 4.00 | 4.00 | 4.00 | 4.00 |
| Phase C | | | | | | |
| Pemulen ™ TR-1 | Acrylates/C10 - 30 alkyl | 0.20 | 0.20 | 0.20 | 0.20 | 0.20 |
| | acrylate cross-polymer | | | | | |
| Phase D | | | | | | |
| Sodium hydroxide | Sodium hydroxide | 0.40 | 0.40 | 0.40 | 0.40 | 0.40 |
| solution 10% | | | | | | |
| Phase E | | | | | | |
| Avn B | | 0.05 | 0.05 | 0.05 | 0.05 | 0.05 |
| Glycerine 99.5% | Glycerine | 1.00 | 1.00 | 1.00 | 1.00 | 1.00 |
| Hydrolite-5 | Pentylene glycol | — | 5.00 | | — | — |
| Hydrolite-6 | 1,2-hexanediol | — | — | 1.00 | 2.00 | — |
| SymDiol 68 | 1,2-hexanediol | — | — | — | — | 1.00 |
| | 1,2-octanediol | | | | | |
| Demineralised water | Water (Aqua) | 2.00 | 2.00 | 2.00 | 2.00 | 2.00 |

| | |
|---|---|
| Sum | 100.00 |
| pH value | 5.5 |

The results are summarised in the following table.

TABLE 7
Formulations (amounts in % by weight)

| Content of | B1 | | | | |
|---|---|---|---|---|---|
| avenanthramide in | Placebo | B3 | B4 | B5 | B6 |
| acceptor solution | (no | 5% | 1% | 2% | 1% |
| (μg/ml) | additive) | Hydrolite-5 | Hydrolite-6 | Hydrolite-6 | SymDiol 68 |
| 0 min | 0.000 | 0.000 | 0.000 | 0.000 | 0.000 |
| 240 min | 0.063 | 0.090 | 0.102 | 0.111 | 0.117 |
| % Modulation of | 0 | +43 | +62 | +76 | +87 |
| permeation vs | | | | | |
| B1 | | | | | |

The results clearly show that adding 1,2-diols potently increases the permeation of Avn B. Modulation by Hydrolite-6 (1,2-hexanediol) was concentration-dependent, giving a 62% increase at a 1% dosage and a 76% increase at a 2% dosage.

SymDiol 68, a 1:1 mixture of 1,2-hexanediol (Hydrolite-6) and 1,2-octanediol (Hydrolite-8), was even more efficient at a 1% dosage than Hydrolite-6 alone at the same dosage, as it enhanced the permeation of Avn B by 87% as compared to 62%.

SymDiol 68 also enhances preservatives in formulations, allowing them to have reduced levels of preservatives, which is particularly beneficial for itchy skin which is to be soothed by applying Avns, since itching is often associated with dry, sensitive or damaged skin.

### Example 4: Comparison of the Permeation Behaviour of Avn A, Avn B, Avn C, and Avn L in Comparison to Dihydroavenantramide D (DhAvn D) in the Absence of a Penetration Enhancer (Amount (μg) of Avns or DhAvnD Per ml Acceptor Liquid Over the Time)

The skin permeation of Avn A, Avn B, Avn C and Avn L in comparison to dihydroavenanthramide D (DhAvn D) from a cosmetic formulation across a stratum corneum-mimicking artificial membrane was evaluated as described above using the Skin PAMPA™ (Parallel Artificial Membrane Permeability Assay) model.

TABLE 8
Formulations (amounts in % by weight)

| | | Avn | Avn | Avn | Avn | DhAvn |
|---|---|---|---|---|---|---|
| Raw material | INCI | A | B | C | L | D |
| Phase A | | | | | | |
| Demineralised | Water (aqua) | ad 100 | | | | |
| water | | | | | | |
| EDTA BD | Disodium EDTA | 0.10 | 0.10 | 0.10 | 0.10 | 0.10 |
| Glycerine 99.5% | Glycerine | 0.50 | 0.50 | 0.50 | 0.50 | 0.50 |
| Phase B | | | | | | |
| PCL Liquid 100 | Cetearyl ethyl hexanoate | 3.00 | 3.00 | 3.00 | 3.00 | 3.00 |
| Lanette O | Cetearyl alcohol | 2.00 | 2.00 | 2.00 | 2.00 | 2.00 |
| Neutral oil | Caprylic acid/capric | 3.00 | 3.00 | 3.00 | 3.00 | 3.00 |
| | triglycerides | | | | | |
| Eutanol G | Octyl dodecanol | 4.00 | 4.00 | 4.00 | 4.00 | 4.00 |
| Phase C | | | | | | |
| Pemulen ™ TR-1 | Acrylates/C10 - 30 alkyl | 0.20 | 0.20 | 0.20 | 0.20 | 0.20 |
| | acrylate cross-polymer | | | | | |
| Phase D | | | | | | |
| Sodium hydroxide | Sodium hydroxide | 0.40 | 0.40 | 0.40 | 0.40 | 0.40 |
| solution 10% | | | | | | |
| Phase E | | | | | | |
| Avn A | | 0.05 | | | | |
| Avn B | | | 0.05 | | | |
| Avn C | | | | 0.05 | | |
| Avn L | | | | | 0.05 | |
| DhAvn D | | | | | | 0.05 |
| Glycerine 99.5% | Glycerine | 1.00 | 1.00 | 1.00 | 1.00 | 1.00 |
| Demineralised | Water (Aqua) | 2.00 | 2.00 | 2.00 | 2.00 | 2.00 |
| water | | | | | | |

| | |
|---|---|
| Sum | 100.00 |
| pH value | 5.5 |

Production Method:

Heat Phase A and B separately to 70° C.; disperse Phase C in B; add Phase BC to Phase A and emulsify using an ULTRA TURRAX® stirrer (2 minutes); allow to cool by using a vane stirrer; add Phase D for neutralising. The formulation was prepared in bulk. The active substances were pre-dissolved in glycerine or water or mixtures thereof and were added to the formulation (15 minutes stirring with a vane stirrer).

Sample Preparation:

After 60, 120 and 240 minutes, approximately 180 μl of each Skin PAMPA™ sample was transferred into an HPLC micro-vial and analysed by HPLC.

The results are summarised in the following table 9.

TABLE 9

| | Dihydroaven- | | | | |
|---|---|---|---|---|---|
| Mean | anthramide D | Avn A | Avn B | Avn L | Avn C |
| Time | (μg per | (μg per | (μg per | (μg per | (μg per |
| [min] | ml) | ml) | ml) | ml) | ml) |
| 0 | 0.000 | 0 | 0 | 0 | 0 |
| 60 | 0.225 | 0.069 | 0.038 | 0 | 0.321 |
| 120 | 0.288 | 0.188 | 0.063 | 0.000 | 0.532 |
| 240 | 0.750 | 0.319 | 0.154 | 0.030 | 1.098 |

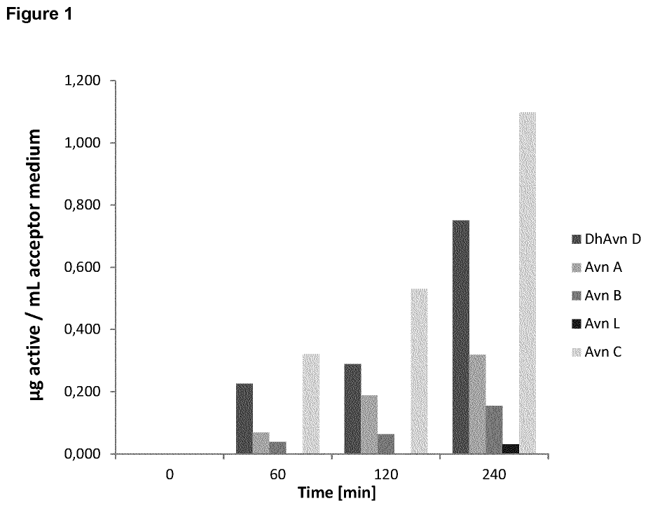

The results are summarized in FIG. 1. As it is obvious from FIG. 1, Avn A<Avn B<Avn L have a weaker penetration behaviour than DhAvn D. However, Avn C penetrates better than DhAvn D.

### Example 5: Modulation of the Penetration Behaviour of Avn L in Comparison to Dihydroavenantramide D (DhAvn D) in the Presence of 1,2-Pentanediol (Hydrolite 5) (3% wt %)

The permeation of Avn L in comparison to dihydroavenanthramide D (DhAvn D) from a cosmetic formulation in the presence of 3% 1,2-pentanediol across a stratum corneum-mimicking artificial membrane was evaluated as described above using the Skin PAMPA™ (Parallel Artificial Membrane Permeability Assay) model.

TABLE 10
Formulations (% parts by weight)

| Raw material | INCI | Avn L | DhAvn D |
|---|---|---|---|
| Phase A | | | |
| Demineralised water | Water (aqua) | Ad 100 | |
| EDTA BD | Disodium EDTA | 0.10 | 0.10 |
| Glycerine 99.5% | Glycerine | 0.50 | 0.50 |
| Phase B | | | |
| PCL Liquid 100 | Cetearyl ethyl hexanoate | 3.00 | 3.00 |
| Lanette O | Cetearyl alcohol | 2.00 | 2.00 |
| Neutral oil | Caprylic acid/capric | 3.00 | 3.00 |
| | triglycerides | | |
| Eutanol G | Octyl dodecanol | 4.00 | 4.00 |
| Phase C | | | |
| Pemulen ™ TR-1 | Acrylates/C10 - 30 alkyl | 0.20 | 0.20 |
| | acrylate cross-polymer | | |
| Phase D | | | |
| Sodium hydroxide | Sodium hydroxide | 0.40 | 0.40 |
| solution 10% | | | |
| Phase E | | | |
| Avn L | | 0.05 | |
| DhAvn D | | | 0.05 |
| Glycerine 99.5% | Glycerine | 1.00 | 1.00 |
| Hydrolite-5 | Pentylene glycol | 3.00 | 3.00 |
| Demineralised water | Water (Aqua) | 2.00 | 2.00 |

| | |
|---|---|
| Sum | 100.00 |
| pH value | 5.5 |

The results are summarised in the following table 11.

TABLE 11

| | |
|---|---|
| Mean | |
| Time | Modulation (%) |

| [min] | Dihydroavenantrahmide D | Avn L |
|---|---|---|
| 0 | — | — |
| 60 | −2 | — |
| 120 | 58 | — |
| 240 | 57 | 125 |

The addition of 1,2-pentanediol (Hydrolite 5) to dihydroavenanthramide D results in an enhancement of the penetration by 57% after 240 min. The addition of 1,2-pentanediol (Hydrolite 5) to avenanthramide L results in an enhancement of the penetration by even 125%.

### Example 6: Formulation Examples

TABLE 12
Perfume oil 1 (PO1; amounts in % by weight)

| Ingredients | Amount |
|---|---|
| ALDEHYDE C14 SO-CALLED | 2 |
| ALLYL AMYL GLYCOLATE 10% DPG | 5 |
| ANISIC ALDEHYDE PURE | 5 |
| APPLE OLIFFAC TYPE | 10 |
| Benzylacetat | 50 |
| BERGAMOT IDENTOIL ® COLOURLESS | 15 |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL BUTYRATE | 2 |
| DIPROPYLENE GLYCOL | 178 |
| EBANOL | 2 |
| ETHYL DECADIENOATE TRANS CIS-2,4 | 2 |
| 10% IPM | |
| FLOROSA | 5 |
| FRAMBINON ® 10% DPG | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 10% DPG | 1 |
| HEXENYL SALICYLATE CIS-3 | 5 |
| HEXYL CINNAMIC ALDEHYDE ALPHA | 70 |
| HEXYL SALICYLATE | 50 |
| HYDROXY CITRONELLAL | 10 |
| ISO E SUPER | 15 |
| ISORALDEINE 70 | 20 |
| LEAFOVERT ® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL ® | 7 |
| STYRALYL ACETATE | 3 |
| TAGETES RCO 10% TEC | 2 |
| TERPINEOL PURE | 20 |
| TETRAHYDROGERANIOL 10% DPG | 5 |
| TONALIDE | 7 |
| VERTOCITRAL 10% DPG | 5 |
| VERTOFIX | 15 |
| Total | 1000 |

TABLE 13
Perfume oil 2 (PO2; amounts in ‰ by weight)

| Ingredients | Amount |
|---|---|
| Acetophenone, 10% in DPG | 10 |
| n-Undecanal | 5 |
| Aldehyde C14, so-called (peach aldehyde) | 15 |
| Allylamyl glycolate, 10% in DPG | 20 |
| Amyl salicylate | 25 |
| Benzyl acetate | 60 |
| Citronellol | 80 |
| d-Limonene | 50 |
| Decenol trans-9 | 15 |
| Dihydromyrcenol | 50 |
| Dimethylbenzylcarbinyl acetate | 30 |
| Diphenyloxide | 5 |
| Eucalyptol | 10 |
| Geraniol | 40 |
| Nerol | 20 |
| Geranium oil | 15 |
| Hexenol cis-3, 10% in DPG | 5 |
| Hexenyl salicylate cis-3 | 20 |
| Indole, 10% in DPG | 10 |
| Alpha-ionone | 15 |
| Beta-ionone | 5 |
| Lilial ® (2-methyl-3-(4-tert-butyl- | 60 |
| phenyl)propanal) | |
| Linalool | 40 |
| Methylphenyl acetate | 10 |
| Phenylethyl alcohol | 275 |
| Styrolyl acetate | 20 |
| Terpineol | 30 |
| Tetrahydrolinalool | 50 |
| Cinnamyl alcohol | 10 |
| Total: | 1000 |

TABLE 14
Perfume oil 3 (PO3; amounts in ‰ by weight)

| Ingredients | Amount |
|---|---|
| Benzyl acetate | 60 |
| Citronellyl acetate | 60 |
| Cyclamenaldehyde (2-methyl-3-(4- | 20 |
| isopropylphenyl)propanal | |
| Dipropylene glycol (DPG) | 60 |
| Ethyllinalool | 40 |
| Florol (2-isobutyl-4-methyltetrahydro-2H-pyran-4-ol) | 30 |
| Globanone ® [(E/Z)-8-cyclohexadecen-1-one] | 180 |
| Hedione ® (methyldihydrojasmonate) | 140 |
| Hexenyl salicylate, cis-3 | 10 |
| Vertocitral (2,4-dimethyl-3-cyclohexenecarboxaldehyde) | 5 |
| Hydratropaldehyde, 10% in DPG | 5 |
| Isodamascone (1-(2,4,4-trimethyl-2-cyclohexen-1-yl)- | 5 |
| 2-buten-1-one, 10% in DPG | |
| Isomuscone (cyclohexadecanone) | 40 |
| Jacinthaflor (2-methyl-4-phenyl-1,3-dioxolane) | 10 |
| Cis-jasmone, 10% in DPG | 20 |
| Linalool | 50 |
| Linalyl acetate | 30 |
| Methyl benzoate, 10% in DPG | 25 |
| para-Methyl cresol, 10% in DPG | 10 |
| Nerol | 20 |
| Phenylpropylaldehyde | 5 |
| 2-Phenylethyl alcohol | 82 |
| Tetrahydrogeraniol | 13 |
| 2,2-Dimethyl-3-cyclohexyl-1-propanol | 80 |
| Total: | 1000 |

TABLE 15
Perfume oil 4 (PO4; amounts in ‰ by weight)

| Ingredients | Amount |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL. bergapten-free | 60 |
| CALONE ® 1951 10% in DPG | 15 |
| COUMARIN | 5 |
| CYCLOGALBANATE ® 10% in DPG | 10 |
| ALPHA -DAMASCONE 1% in DPG | 20 |
| DIHYDROMYRCENOL | 10 |
| ETHYL LINALOOL | 75 |
| ETHYL LINALYLACETATE | 50 |
| ETHYL MALTOL 1% in DEP | 10 |
| ETHYLENE BRASSYLATE (MACRO) | 80 |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDE ® (ALICYC) | 30 |
| HEXENYLSALICYLATE CIS-3 | 20 |
| ISO E SUPER ® | 40 |
| LEAFOVERT ® 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN 10% in DEP | 20 |
| DIPROPYLENE GLYCOL (DPG) | 50 |
| TOTAL | 1000 |

TABLE 16
Perfume oil 5 (PO5; amounts in ‰ by weight)

| Ingredients | Amount |
|---|---|
| AMAROCITE ® | 10 |
| AMBROCENIDE ® 10% in DPG | 5 |
| AMBROXIDE | 15 |
| AURELIONE ® (7/8-Cyclohexadecenone) (MACRO) | 70 |
| BERGAMOT OIL. bergapten-free | 90 |
| CALONE ® 1951 10% in DPG | 20 |
| CARAWAY OIL | 10 |
| CITRAL | 20 |
| COUMARIN | 10 |
| ALPHA-DAMASCONE 1% in DPG | 15 |
| DIHYDROMYRCENOL | 70 |
| ESTRAGON OIL | 10 |
| ETHYL LINALOOL | 100 |
| ETHYL LINALYLACETATE | 90 |
| EUGENOL | 10 |
| EVERNYL ® | 5 |
| FRUCTATE ® | 5 |
| GERANIUM OIL | 5 |
| HEDIONE ® HC/30 | 100 |
| HELIONAL ® | 10 |
| INDOLE 10% in DPG | 5 |
| ISO E SUPER ® | 100 |
| KEPHALIS ® | 5 |
| LAVENDER OIL | 40 |
| CITRUS OIL | 80 |
| LILIAL ® | 30 |
| MANDARIN OIL | 20 |
| MUSCENONE (MACRO) | 5 |
| SANDRANOL ® | 10 |
| VANILLIN 10% in DPG | 5 |
| DIPROPYLENE GLYCOL | 30 |
| TOTAL | 1000 |

The above perfume oils PO1, PO2, PO3, PO4, or PO5 were worked separately in each case into the preparations presented below.

Cosmetic preparations/formulations (amounts in % by weight for all preparations/formulations).

TABLE 17
Cream, o/w

| Ingredients | INCI | Amount |
|---|---|---|
| Dracorin ® CE | Glyceryl Stearate Citrate | 1.0 |
| Lanette ® O | Cetearyl Alcohol | 2.0 |
| Cutina ® GMS-V | Glyceryl Stearate | 1.0 |
| Tegosoft ® MM | Myristyl Myristate | 1.0 |
| Xiameter ® PMX-0246 | Cyclohexasiloxane, | 0.5 |
| | Cyclopentasiloxane | |
| Dragoxat ® 89 | Ethylhexyl Isononanoate | 2.0 |
| PCL-Liquid 100 | Cetearyl Ethylhexanoate | 4.0 |
| Neutral Oil | Caprylic/Capric Triglyceride | 4.0 |
| Carbopol ® Ultrez 21 | Acrylates/C10-30 Alkyl Acrylate | 0.2 |
| | Crosspolymer | |
| Keltrol ® CG-T | Xanthan Gum | 0.1 |
| Water | Water (Aqua) | ad 100 |
| Glycerol 99.5 P. | Glycerol | 3.0 |
| Hydrolite CG | Caprylyl Glycol | 0.2 |
| Hydrolite-5 Greeen | Pentylene Glycol | 2.0 |
| 1,2-Propylene Glycol 99 | Propylene Glycol | 2.0 |
| P GC | | |
| Sodium Benzoate | Sodium Benzoate | 0.1 |
| Sodium Hydroxide 10% | Sodium Hydroxide | 0.5 |
| solution | | |
| Perfume oil PO1, PO2, | Perfume | 0.3 |
| PO3, PO4, or PO5 | | |
| Euxyl ® K702 | Dehydroacetic Acid, Benzoic Acid, | 0.3 |
| | Phenoxyethanol, Polyaminopropyl | |
| | Biguanide, Ethylhexylglycerin | |
| Avenanthramide A, B, C | Aqua, Glycerine, Avena Sativa | 1.0 |
| and L ≥ 100 ppm (oat | (Oat) Kernel Extract | |
| extract fraction) in sum in | | |
| glycerine/water | | |

TABLE 18
Hand and body cream

| Ingredients | INCI | Amount |
|---|---|---|
| Dracorin ® GOC | Glyceryl Oleate Citrate, | 2.0 |
| | Caprylic/Capric Triglyceride | |
| PCL-Solid | Stearyl Heptanoate, Stearyl | 2.5 |
| | Caprylate | |
| Lanette ® O | Cetearyl Alcohol | 1.5 |
| Cutina ® GMS-V | Glyceryl Stearate | 1.0 |
| Dragoxat ® 89 | Ethylhexyl Isononanoate | 3.0 |
| PCL-Liquid 100 | Cetearyl Ethylhexanoate | 7.0 |
| Isodragol ® | Triisononanoin | 4.0 |
| Xiameter ® PMX-0345 | Cyclopentasiloxane (and) | 0.5 |
| Cyclosiloxane | Cyclohexasiloxane | |
| Water | Water (Aqua) | ad 100 |
| Carbopol ® Ultrez 21 | Acrylates/C10-30 Alkyl Acrylate | 0.2 |
| | Crosspolymer | |
| Keltrol ® CG-RD | Xanthan Gum | 0.1 |
| Glycerol 85 P, | Glycerol | 3.0 |
| DragoBetaGlucan | Water (Aqua), Butylene Glycol, | 1.5 |
| | Glycerol, Avena Sativa (Oat) | |
| | Kernel Extract | |
| Potassium Sorbate | Potassium Sorbate | 0.1 |
| Hydrolite-6 | 1,2 Hexanediol | 1.0 |
| Sodium Hydroxide 10% | Sodium Hydroxide | 0.5 |
| solution | | |
| Perfume oil PO1, PO2, | Fragrance | 0.2 |
| PO3, PO4, or PO5 | | |
| Avenanthramide L | Avenanthramide L | 0.1 |
| SymSave H | Hydroxyacetophenone | 0.5 |

TABLE 19
Daily face cream, (SPF 20)

| Ingredients | Amount |
|---|---|
| SymOcide PH | 1 |
| Phenoxyethanol, Hydroxyacetophenone, Caprylyl | |
| Glycol, Water (Aqua) | |
| Ascorbyl Palmitate | 0.1 |
| Ascorbyl Palmitate | |
| Biotive L-Arginine | 0.2 |
| Arginine | |
| Buriti oil | 1 |
| Mauritia Flexuosa Fruit Oil | |
| Cocoa butter | 2 |
| Theobroma Cacao (Cocoa) Seed Butter | |
| Dimethicone | 0.5 |
| Dimethicone | |
| Disodium EDTA | 0.1 |
| Disodium EDTA | |
| Dragosantol 100 | 0.1 |
| Bisabolol | |
| Dragoxat 89 | 5 |
| Ethylhexyl Isononanoate | |
| Emulsiphos | 2 |
| Potassium Cetyl Phosphate, Hydrogenated Palm | |
| Glycerides | |
| Extrapone Corail | 1 |
| Glycerin, Aqua, Hydrolyzed Corallina Officinalis | |
| Glycerin | 3 |
| Glycerin | |
| Isoadipate | 5 |
| Diisopropyl Adipate | |
| Jojoba Wax Flakes | 1 |
| Hydrogenated Jojoba Oil | |
| Keltrol CG-T | 0.1 |
| Xanthan Gum | |
| Lanette O | 5 |
| Cetearyl Alcohol | |
| Lanette 16 | 1 |
| Cetyl Alcohol | |
| Lanette 22 | 1 |
| Behenyl Alcohol | |
| Neo Heliopan 357 | 3 |
| Butyl Methoxydibenzoylmethane | |
| Neo Heliopan HMS | 10 |
| Homosalate | |
| Neo Heliopan Hydro used as a 25% aq, Solution | 8 |
| neutralized by arginine | |
| Phenylbenzimidazole Sulfonic Acid | |
| Neo Heliopan OS | 5 |
| Ethylhexyl Salicylate | |
| Orgasol Caresse | 1 |
| Polyamide-5 | |
| Perfume oil PO1, PO2, PO3, PO4, or PO5 | 0.1 |
| Shea butter | 3 |
| Butyrospermum Parkii (Shea) Butter | |
| Simugel EG | 1 |
| Sodium Acrylate/Sodium Acryloyldimethyl Taurate | |
| Copolymer. Isohexadecane. Polysorbate 80 | |
| SymFinity 1298 | 0.1 |
| Echinacea Purpurea Extract | |
| SymDiol 68 | 0.5 |
| 1.2 Hexanediol. Caprylyl Glycol | |
| SymMatrix | 0.1 |
| Maltodextrin, Rubus Fructicosus (Blackberry) Leaf | |
| Extract | |
| SymSitive 1609 | 1 |
| Pentylene Glycol, 4-t-Butylcyclohexanol | |
| Tegosoft TN | 4 |
| C12-15 Alkyl Benzoate | |
| Avenanthramide L | 0.001 |
| SymSave H (Hydroxyacetophenone) | 0.1 |
| Water | ad 100 |
| Aqua | |

TABLE 20
Night cream, w/o

| Ingredients | INCI | Amount |
|---|---|---|
| Avenanthramide A | Avenanthramide L | 0.005 |
| SymSave H | Hydroxyacetophenone | 0.3 |
| Avenanthramide B | Avenanthramide B | 0.005 |
| Aloe Vera Gel | Water (Aqua), Aloe | 3.0 |
| Concentrate 10/1 * | Barbadensis Leaf Juice | |
| Alugel 34 TH | Aluminium Stearate | 1.0 |
| Dragosan W/O P* | Sorbitan Isostearate, | 6.0 |
| | Hydrogenated Castor Oil, | |
| | Ceresin, Beeswax (Cera Alba) | |
| Dragosantol ® 100* | Bisabolol | 0.2 |
| Extrapone ® Witch | Propylene Glycol, Hamamelis | 1.0 |
| Hazel Distillate | Virginiana (Witch Hazel) Water, | |
| colourless | Water (Aqua), Hamamelis | |
| | Virginiana (Witch Hazel) Extract | |
| Perfume oil PO1, PO2, | Fragrance | 0.4 |
| PO3, PO4, or PO5 | | |
| Glycerol 85% | Glycerin | 2.0 |
| Hydrolite-5 | Pentylene Glycol | 5.0 |
| Karion F | Sorbitol | 2.0 |
| Magnesium Chloride | Magnesium Chloride | 0.7 |
| PCL Liquid 100 | Cetearyl Ethylhexoate | 12.0 |
| Retinyl Palmitate in Oil | Retinyl Palmitate | 0.2 |
| Sun Flower Oil | Helianthus Annuus (Sunflower) | 5.0 |
| | Seed Oil | |
| Sweet Almond Oil | Prunus dulcis | 5.0 |
| Sym Matrix ® | Maltodextrin, Rubus Fruticosus | 1.0 |
| | (Blackberry) Leaf Extract | |
| SymOcide PS | Phenoxyethanol, Decylene | 0.5 |
| | glycol, 1,2-Hexanediol | |
| SymVital ® AgeRepair | Zingiber Officinale (Ginger) | 0.1 |
| | Root Extract | |
| Tocopherol Acetate | Tocopheryl Acetate | 3.0 |
| Water (demineralized) | Water (Aqua) | ad 100 |

TABLE 21
Body lotion

| Ingredients | Amount |
|---|---|
| Cetearyl Alcohol | 2.0 |
| Ethylhexyl Isononanoate | 5.0 |
| Cetearyl Ethylhexanoate, Isopropyl Myristate | 3.0 |
| Glyceryl Oleate Citrate, Caprylic/Capric Triglyceride | 4.0 |
| (Dracorin GOC) | |
| Water (Aqua) | ad 100 |
| Pentylene Glycol (Hyddrolite-5 Green) | 3.0 |
| Carbomer | 0.3 |
| Sodium Benzoate | 0.1 |
| Propylene Glycol | 5.0 |
| Sodium Hydroxide 30% solution | 0.3 |
| Perfume oil PO1, PO2, PO3, PO4, or PO5 | 0.3 |
| 4-Hydroxyacetophenone (SymSave H) | 0.3 |
| Avenanthramide L | 0.2 |
| Avenanthramide B | 0.2 |

TABLE 22
Antibacterial body lotion, sprayable

| Ingredients | INCI | Amount |
|---|---|---|
| Avenanthramide | Aqua, Glycerine, Avena Sativa | 1.5 |
| A, B, C and L ≥ | (Oat) Kernel Extract | |
| 100 ppm (oat extract | | |
| fraction) in sum in | | |
| glycerine/water | | |
| SYmSave H | Hydroxyacetophenone | 0.5 |
| Triethyl Citrate | Triethyl Citrate | 0.2 |
| 2,4-Hexadienoic acid, | Sorbic acid, potassium salt | 0.2 |
| potassium salt | | |
| Dow Corning 345 Fluid | Cyclomethicone | 0.5 |
| Dracorin ® GOC | Glyceryl Oleate Citrate, | 2.0 |
| | Caprylic/Capric Triglyceride | |
| Drago-Calm | Water, Glycerin, Avena Sativa | 1.0 |
| | (Oat) Kernel Extract | |
| Dragosantol ® 100* | Bisabolol | 0.1 |
| Perfume oil PO1, PO2, | Fragrance | 0.3 |
| PO3, PO4, or PO5 | | |
| Hydrolite ®-5 | Pentylene Glycol | 5.0 |
| Neutral Oil | Caprylic/Capric Triglyceride | 4.0 |
| Paraffin Oil | Mineral Oil | 4.0 |
| PCL Liquid 100 | Cetearyl Ethylhexoate | 7.0 |
| Pemulen TR-2 | Acrylates/C10-30 Alkyl Acrylate | 0.2 |
| | Crosspolymer | |
| Sodium Hydroxide | Sodium Hydroxide | 0.4 |
| (10% solution) | | |
| SymDeo ® MPP | Dimethyl Phenylbutanol | 0.5 |
| SymRelief ® 100 | Bisabolol, Zingiber Officinale | 0.1 |
| | (Ginger) Root Extract | |
| Water (demineralized) | Water (Aqua) | ad 100 |

TABLE 23
Aseptic wound cream

| Ingredients | Amount |
|---|---|
| Sorbitan Isostearate, Hydrogenated Castor Oil, Ceresin, | 6.0 |
| Beeswax (Cera Alba) | |
| Petrolatum | 21.0 |
| Cera Alba | 5.0 |
| Cetearyl Alcohol | 7.0 |
| Prunus Dulcis | 7.0 |
| Lanolin | 5.0 |
| Paraffinum Liquidum | 12.0 |
| Perfume oil PO1, PO2, PO3, PO4, or PO5 | 0.3 |
| Water (Aqua) | ad 100 |
| Panthenol | 7.0 |
| Magnesium Sulfate | 0.7 |
| 1,2-Hexanediol (Hydrolite-6 ) | 1.0 |
| Tocopheryl Acetate | 1.0 |
| Octenidine dihydrochloride | 0.1 |
| Phenoxyethanol | 0.5 |
| Avenanthramide L | 0.001 |
| Hydroxyacetophenone (SymSave H) | 0.5 |

TABLE 24
Anti acne balm

| Ingredients | INCI | Amount |
|---|---|---|
| Avenanthramide L | Avenanthramide L | 0.0005 |
| Avenanthramide A | Avenanthramide A | 0.005 |
| Hydrolite-5 Green | Pentylene Glycol | 2.0 |
| SymSave H | Hydroxyacetophenone | 0.5 |
| Abil 350 | Dimethicone | 1.0 |
| Allantoin | Allantoin | 0.1 |
| Aloe Vera Gel | Water (Aqua), Aloe | 3.0 |
| Concentrate 10/1 * | Barbadensis Leaf Juice | |
| Azelaic Acid | Azelaic Acid | 5.0 |
| Cetiol OE | Dicaprylyl Ether | 4.0 |
| Cetiol SB 45 | Butyrospermum Parkii (Shea | 1.0 |
| | Butter) | |
| D-Panthenol | Panthenol | 1.0 |
| SymClariol | Decylene Glycol | 0.1 |
| Emulsiphos ® | Potassium Cetyl Phosphate, | 2.0 |
| | Hydrogenated Palm Glycerides | |
| Perfume oil PO1, PO2, | Fragrance | 0.2 |
| PO3, PO4, or PO5 | | |
| Frescolat ®ML cryst, | Menthyl Lactate | 0.8 |
| Glycerol 85% | Glycerin | 4.0 |
| Hydroviton ® PLUS | Water, Pentylene Glycol, | 1.0 |
| | Glycerin, Fructose, Urea, Citric | |
| | Acid, Sodium Hydroxide, | |
| | Maltose, Sodium PCA, Sodium | |
| | Chloride, Sodium Lactate, | |
| | Trehalose, Allantoin, Sodium | |
| | hyaluronate, Glucose | |
| Lara Care A-200 | Galactoarabinan | 0.3 |
| Pemulen TR-2 | Acrylates/C10-30 Alkyl Acrylate | 0.2 |
| | Crosspolymer | |
| Sodium Hydroxide | Sodium Hydroxide | 0.4 |
| (10% solution) | | |
| SymTriol | Caprylyl glycol, 1,2-Hexanediol, | 1.0 |
| | Methylbenzyl alcohol | |
| Tegosoft TN | C12-15 Alkyl Benzoate | 5.0 |
| Tocopherol Acetate | Tocopheryl Acetate | 0.5 |
| Water (demineralized) | Water (Aqua) | ad 100 |

TABLE 25
Barrier repair cream

| Ingredients | INCI | Amount |
|---|---|---|
| Avenanthramide L | Avenanthramide L | 0.001 |
| Avenanthramide B | Avenanthramide B | 0.003 |
| SymSave H | Hydroxyacetophenone | 0.6 |
| Abil 350 | Dimethicone | 0.5 |
| Allantoin | Allantoin | 0.25 |
| Ceramide BIO* | Cetylhydroxyproline | 0.5 |
| | Palmitamide | |
| Dracorin ® CE | Glyceryl Stearate Citrate | 1.5 |
| Dragoxat ® 89 | Ethylhexyl Ethylisononan-oate | 2.0 |
| Emulsiphos ® | Potassium Cetyl Phosphate, | 2.0 |
| | Hydrogenated Palm Glycerides | |
| Extrapone ® Rosemary | Glycerin, Water (Aqua), | 0.5 |
| GW | Rosmarinus officinalis | |
| | (Rosemary) Leaf Extract | |
| Perfume oil PO1, PO2, | Fragrance | 0.1 |
| PO3, PO4, or PO5 | | |
| Glycerol 85% | Glycerin | 3.0 |
| Glyceryl Stearate | Glyceryl Stearate | 2.0 |
| Hydroviton ® 24 | Water, Glycerin, Sodium | 1.0 |
| | Lactate, TEA Lactate, Serine, | |
| | Lactic Acid, Urea, Sorbitol, | |
| | Sodium Chloride, Lauryl | |
| | Diethylenedi-aminoglycine, | |
| | Lauryl Aminopropyl-glycine, | |
| | Allantoin | |
| Hydrolite- 8 | Caprylyl Glycol | 0.5 |
| Isodragol ® | Triisononanoin | 3.0 |
| Lanette O | Cetearyl Alcohol | 2.0 |
| NaOH | Sodium Hydroxide | 0.3 |
| 10% solution) | | |
| Neutral Oil | Caprylic/Capric Triglyceride | 10.0 |
| SymCalmin ® | Pentylene Glycol, Butylene | 1.0 |
| | Glycol, Hydroxyphenyl | |
| | Propamidobenzoic Acid | |
| SymRepair ® 100 | Hexyldecanol, Bisabolol, | 2.0 |
| | Cetylhydroxyproline | |
| | Palmitamide, Stearic Acid, | |
| | Brassica Campestris | |
| | (Rapeseed) Sterols | |
| SymTriol | Caprylyl glycol, 1,2-Hexanediol, | 1.0 |
| | Methylbenzyl alcohol | |
| Tegosoft PC 31 | Polyglyceryl 3- Caprate | 0.3 |
| Tocopherol Acetate | Tocopheryl Acetate | 0.3 |
| Water (demineralized) | Water (Aqua) | ad 100 |

TABLE 26
Skin soothing lotion

| Ingredients | INCI | Amount |
|---|---|---|
| Avenanthramide L | Avenanthramide L | 0.001 |
| SymSave H | Hydroxyacetophenone | 0.3 |
| Hydrolite-5 Green | Pentylene Glycol | 1.5 |
| Avenanthramide A | Avenanthramide A | 0.01 |
| Abil 350 | Dimethicone | 2.0 |
| Allantoin | Allantoin | 0.2 |
| Carbopol Ultrez-10 | Carbomer | 0.1 |
| Ceramide BIO* | Cetylhydroxyproline | 0.1 |
| | Palmitamide | |
| Citric Acid | Citric Acid | 0.4 |
| (10% solution) | | |
| Emulsiphos ® | Potassium Cetyl Phosphate, | 2.0 |
| | Hydrogenated Palm Glycerides | |
| Extrapone ® Green Tea | Glycerin, Water (Aqua), | 0.2 |
| GW | Camellia Sinensis Leaf Extract | |
| Extrapone ® Rosemary | Glycerin, Water (Aqua), | 0.3 |
| GW | Rosmarinus officinalis | |
| | (Rosemary) Leaf Extract | |
| Perfume oil PO1, PO2, | Fragrance | 0.3 |
| PO3, PO4, or PO5 | | |
| Glycerol 85% | Glycerin | 2.0 |
| Glyceryl Stearate | Glyceryl Stearate | 2.0 |
| Isodragol ® | Triisononanoin | 2.0 |
| Keltrol RD | Xanthan Gum | 0.1 |
| Lanette O | Cetearyl Alcohol | 3.0 |
| Neo PCL wssl, N | Trideceth-9, PEG-5 | 1.0 |
| | Ethylhexanoate, Water | |
| PCL Liquid 100 | Cetearyl Ethylhexanoate | 5.0 |
| PCL Solid | Stearyl Heptanoate, Stearyl | 2.0 |
| | Caprylate | |
| Propylene Glycol | Propylene Glycol | 5.0 |
| Sodium Hydroxide | Sodium Hydroxide | 0.3 |
| (10% solution) | | |
| SymCalmin ® | Pentylene Glycol, Butylene | 2.0 |
| | Glycol, Hydroxyphenyl | |
| | Propamidobenzoic Acid | |
| SymMatrix ® | Maltodextrin, Rubus Fruticosus | 0.1 |
| | (Blackberry) Leaf Extract | |
| 2-Phenoxyethyl Alcohol | Phenoxyethanol | 0.4 |
| SymSitive ®1609 | Pentylene Glycol, 4-t- | 1.5 |
| | Butylcyclohexanol | |
| Water (demineralized) | Water (Aqua) | ad 100 |

TABLE 27
Baby nappy ash cream, w/o

| Ingredients | Amount |
|---|---|
| SymOcide PH | 1 |
| Phenoxyethanol, Hydroxyacetophenone, Caprylyl | |
| Glycol, Water (Aqua) | |
| Cupuaçu butter | 1 |
| Theobroma Grandiflorum Seed Butter | |
| Cutina HR Powder | 1.5 |
| Hydrogenated Castor Oil | |
| Dehymuls PGPH | 5 |
| Polyglyceryl-2 Dipolyhydroxystearate | |
| Glycerin | 5 |
| Glycerin | |
| Jojoba oil | 5 |
| Simmondsia Chinensis (Jojoba) Seed Oil | |
| Magnesium Sulfate Hepta Hydrate | 0.5 |
| Magnesium Sulfate | |
| Monomuls 90-O18 | 1 |
| Glyceryl Oleate | |
| Neutral oil | 8 |
| Caprylic/capric triglyceride | |
| PCL Liquid 100 | 5 |
| Cetearyl Ethylhexanoate | |
| SymCalmin | 1 |
| Butylene Glycol, Pentylene Glycol, Hydroxyphenyl | |
| Propamidobenzoic Acid | |
| Tamanu oil | 0.2 |
| Calophyllum Inophyllum Seed Oil | |
| Tetrasodium EDTA | 0.1 |
| Tetrasodium EDTA | |
| Titan dioxide | 4 |
| Titan dioxide | |
| Water | ad 100 |
| Aqua | |
| Wheat germ oil | 2 |
| Triticum Vulgare (Wheat) Germ Oil | |
| Zinc oxide | 10 |
| Zinc oxide | |
| Hydrolite-8 | 0.3 |
| Caprylyl Glycol | |
| Symsave H | 0.5 |
| Hydroxyacetophenone | |
| Avenanthramide L | 0.0003 |
| Avenanthramide A | 0.002 |

TABLE 28
Skin lightening day cream, o/w

| Ingredients | INCI | Amount |
|---|---|---|
| Avenanthramide L | Avenanthramide L | 0.001 |
| Avenanthramide B | Avenanthramide B | 0.002 |
| Avenanthramide A | Avenanthramide A | 0.002 |
| Hydrolite-5 Green | Pentylene Glycol | 1.5 |
| SymSave H | Hydroxyacetophenone | 0.5 |
| Abil 350 | Dimethicone | 0.5 |
| Dracorin ® CE | Glyceryl Stearate Citrate | 2.5 |
| Dracorin ® GOC | Glyceryl Oleate Citrate, | 0.5 |
| | Caprylic/Capric Triglyceride | |
| Drago-Beta-Glucan | Water (Aqua), Butylene Glycol, | 0.3 |
| | Glycerin, Avena Sativa (Oat), | |
| | Kernel Extract | |
| Dragosantol ® 100* | Bisabolol | 0.2 |
| Perfume oil PO1, | Fragrance | 0.1 |
| PO2, PO3, PO4, or | | |
| PO5 | | |
| Frescolat ®MGA | Menthone Glycerol Acetal | 0.5 |
| Glycerol 85% | Glycerin | 3.0 |
| Isopropyl Palmitate | Isopropyl Palmitate | 4.0 |
| Keltrol RD | Xanthan Gum | 0.2 |
| Lanette 16 | Cetyl Alcohol | 1.0 |
| Neo Heliopan ® AV | Ethylhexyl Methoxy-cinnamate | 5.0 |
| Neutral Oil | Caprylic/Capric Triglyceride | 6.0 |
| PCL Liquid 100 | Cetearyl Ethylhexoate | 3.0 |
| Sodium Benzoate | Sodium Benzoate | 0.1 |
| Symdiol ®68T | 1,2-Hexanediol, Caprylylglycol, | 0.5 |
| | Tropolone | |
| SymVital ® | Zingiber Officinale (Ginger) Root | 0.1 |
| AgeRepair | Extract | |
| SymWhite ®377 | Phenylethyl Resorcinol | 0.5 |
| Water | Water (Aqua) | ad 100 |
| (demineralized) | | |

TABLE 29
Shampoo

| Ingredients | Amount |
|---|---|
| Antil 127 | 0.5 |
| PEG-120 Methyl Glucose Dioleate | |
| Brazilian nut oil | 0.5 |
| Bertholletia Excelsa Seed Oil | |
| Cocamidopropyl Betaine 38% | 5 |
| Cocamidopropyl Betaine | |
| Octopirox | 0.3 |
| Piroctone olamine | |
| Dragoderm | 0.5 |
| Glycerin. Triticum Vulgare Gluten. Aqua | |
| Fragrance | 0.5 |
| Perfum | |
| Glycerin | 0.5 |
| Glycerin | |
| Jojoba oil | 0.5 |
| Simmondsia Chinensis (Jojoba) Seed Oil | |
| Marlinat 242/90 M | 15 |
| MIPA Laureth Sulfate. Propylene Glycol | |
| Marlowet CG | 2 |
| PEG-18 Castor Oil Dioleate | |
| Plantacare 1200 UP | 0.5 |
| Lauryl Glucoside | |
| Polyquaternium-10 | 0.3 |
| Polyquaternium-10 | |
| Sodium Chloride | 1.5 |
| Sodium Chloride | |
| SymCalmin | 1 |
| Butylene Glycol, Pentylene Glycol, Hydroxyphenyl | |
| Propamidobenzoic Acid | |
| SymOcide PS | 0.8 |
| Phenoxyethanol, Decylene Glycol, 1,2-Hexanediol | |
| Avenanthramide L | 0.0001 |
| Avenanthramide A | 0.002 |
| SymSave H | 0.5 |
| Hydroxyacetophenone | |
| Water | ad 100 |
| Aqua | |

TABLE 30
Anti dandruff shampoo

| Ingredients | INCI | Amount |
|---|---|---|
| Avenanthramide L | Avenanthramide L | 0.001 |
| Avenanthramide A | Avenanthramide A | 0.15 |
| Avenanthramide B | Avenanthramide B | 0.05 |
| SymSave H | Hdroxyacetophenone | 1.0 |
| Aloe Vera Gel | Water (Aqua), Aloe | 0.5 |
| Concentrate 10/1 * | Barbadensis Leaf Juice | |
| Abrasive/Exfoliant | Perlite | 0.3 |
| Cellulose fibre | Microcrystalline Cellulose | 0.1 |
| Avocado oil | Persea Gratissima (Avocado) | 0.5 |
| | Oil | |
| Citric Acid 10% sol. | Citric Acid | 0.3 |
| Comperlan 100 | Cocamide MEA | 0.5 |
| Crinipan AD | Climbazole | 0.2 |
| Dragoderm ® | Glycerin, Triticum Vulgare | 2.0 |
| | (Wheat) Gluten, Water (Aqua) | |
| Perfume oil PO1, PO2, | Fragrance | 0.5 |
| PO3, PO4, or PO5 | | |
| Genapol LRO liquid | Sodium Laureth Sulfate | 37.0 |
| Merquat 550 | Polyquaternium-7 | 0.5 |
| Xylityl Caprylate | Xylityl Caprylate | 0.5 |
| Sodium Chloride | Sodium Chloride | 1.0 |
| Hydrolite-5 Green | Pentylene Glycol | 0.5 |
| Tego Betain L7 | Cocamidopropyl Betaine | 6.0 |
| Water (demineralized) | Water (Aqua) | ad 100 |

TABLE 31
2-in-1 Shampoo

| Ingredients | INCI Name | Amount |
|---|---|---|
| Deionized water | Water | ad 100 |
| Shea butter | Butyrospermum Parkii (Shea) | 0.1 |
| | Butter | |
| SymSave H | Hydroxyacetophenone | 0.5 |
| SymDiol 68 | 1.2 Hexanediol, Caprylyl | 0.5 |
| | Glycol | |
| Plantacare PS 10 | Sodium Laureth Sulfate, Lauryl | 20.0 |
| | Glucoside | |
| Euperlan PK 771 | Glycol Distearate, Sodium | 6.0 |
| | Lauryl Sulfate, Cocamide | |
| | MEA, Laureth-10 | |
| Sodium chloride | Sodium Chloride | 1.4 |
| Citric acid | Citric acid | 0.1 |
| monohydrate | | |
| crystalline | | |
| Perfume oil PO1, PO2, | Fragrance | 0.5 |
| PO3, PO4, or PO5 | | |
| Zinc Omadine | Zinc pyrithione | 0.10 |
| Avenanthramide L | Avenanthramide L | 0.001 |
| Avenanthramide B | Avenanthramide B | 0.002 |

TABLE 32
Body wash

| Ingredients | INCI | Amount |
|---|---|---|
| Lumerol K 28 | Disodium Laureth | 33.0 |
| | Sulfosuccinate, | |
| | Cocamidopropyl Betaine, | |
| | Magnesium Lauryl Sulfate | |
| Amphotensid B 4 | Cocamidopropyl Betaine | 10.0 |
| Pearly Gloss | MIPA-Pareth-25 Sulfate, | 4.0 |
| | Glycol Stearate | |
| Sodium Chloride | Sodium Chloride | 2.0 |
| Avocado oil | Persea Gratissima (Avocado) | 3.0 |
| | Oil | |
| SymSave H | Hydroxyacetophenone | 0.8 |
| Hydrolite-6 | 1,2 Hexanediol | 1.0 |
| Water | Water | ad 100 |
| Perfume oil PO1, PO2, | Fragrance | 0.5 |
| PO3, PO4, or PO5 | | |
| Glyceryl | Glyceryl caprylate | 0.15 |
| monocaprylate | | |
| Avenanthramide A | Avenanthramide A | 0.1 |
| Avenanthramide L | Avenanthramide L | 0.15 |

TABLE 33
Shower gel

| Ingredients | INCI | Amount |
|---|---|---|
| Deionized water | Water | ad 100 |
| Shea butter | Butyrospermum Parkii (Shea) | 1.0 |
| | Butter | |
| Plantacare PS 10 | Sodium Laureth Sulfate, Lauryl | 20.0 |
| | Glucoside | |
| Hydrolite-6 | 1.2 Hexanediol | 0.5 |
| Dehydroacetic acid | Dehydroacetic acid | 0.2 |
| SymSave H | Hydroxyacetophenone | 0.3 |
| Sodium chloride | Sodium Chloride | 1.4 |
| Citric acid | Citric Acid | 1.3 |
| monohydrate | | |
| crystalline | | |
| Perfume oil PO1, PO2, | Fragrance | 0.6 |
| PO3, PO4, or PO5 | | |
| Avenanthramide L | Avenanthramide L | 0.1 |
| Symlite G 8 | Glyceryl caprylate | 0.3 |
| Avenanthramide A | Avenanthramide A | 0.2 |

TABLE 34
Intimate wash

| Ingredients | INCI | Amount |
|---|---|---|
| Tegobetaine HS | Cocamidopropyl Betaine, | 15.0 |
| | Glyceryl Laurate | |
| Tagat L 2 | PEG-20 Glyceryl Laurate | 2.0 |
| Arlacide G | Chlorhexidine Digluconate | 0.1 |
| Rewoquat B 50 | Benzalkonium Chloride | 0.1 |
| Lactic Acid. 80% | Lactic Acid | 0.1 |
| euxyl ® K700 | Potassium Sorbate, Benzyl | 0.3 |
| | Alcohol. Phenoxyethanol | |
| Water | Water | ad 100 |
| Perfume oil PO1, PO2, | Fragrance | 0.2 |
| PO3, PO4, or PO5 | | |
| Hydrolite-5 Green | Pentylene Glycol | 3.0 |
| SymSave H | Hydroxyacetophenone | 0.3 |
| Avenanthramide L | Avenanthramide L | 0.001 |
| Avenanthramide A | Avenanthramide A | 0.002 |

TABLE 35
Liquid soap, transparent

| Ingredients | INCI | Amount |
|---|---|---|
| Tagat O 2 | PEG-20 Glyceryl Oleate | 2.5 |
| Coconut oil | Cocamide DEA | 5.0 |
| diethanolamine | | |
| condensate | | |
| Abil B 8842 | Cyclomethicone | 0.5 |
| Sodium | Sodium Laureth Sulfate | 35.0 |
| laurylethersulfate. 28% | | |
| Tego-Betaine L7 | Cocamidopropyl Betaine | 5.0 |
| SymSave H | Hydroxyacetophenone | 0.5 |
| Soap. 25% | Coconut acid, Potassium salt, | 20.0 |
| | Potassium Oleate | |
| Perfume oil PO1, PO2, | Fragrance | 0.4 |
| PO3, PO4, or PO5 | | |
| Hydrolite-5 Green | Pentylene Glycol | 5.0 |
| Avenanthramide L | Hydroxypropyl caprylate | 0.15 |
| Water | Water | ad 100 |

TABLE 36
Syndet soap, liquid

| Ingredients | INCI | Amount |
|---|---|---|
| Elfan OS 46 | Sodium Olefin C14-C16 | 35.5 |
| | Sulfonate | |
| Armoteric LB | Lauryl Betaine | 8.0 |
| Euperlan PK 3000 OK | Glycol Distearate, Glycerin, | 10.0 |
| | Laureth-4, Cocamidopropyl | |
| | Betaine | |
| Elfacos GT 282 L | Talloweth-60 Myristyl Glycol | 3.0 |
| PCL-Liquid 100 | Cetearyl Ethylhexanoate | 4.0 |
| Perfume oil PO1, PO2, | Fragrance | 0.4 |
| PO3, PO4, or PO5 | | |
| SymDiol 68 | 1,2 Hexanediol, Caprylyl | 1.0 |
| | Glycol | |
| SymSave H | 4-Hydroxyacetophenone | 0.6 |
| Avenanthramide L | Avenanthramide L | 0.5 |
| Avenanthramide A | Avenanthramide A | 0.5 |
| Water | Water | ad 100 |

TABLE 37
Anti-acne wash

| Ingredients | Amount |
|---|---|
| Water (Aqua) | ad 100 |
| Polyquaternium-7 | 0.5 |
| Cocamidopropyl Betaine | 9.0 |
| Coco Glucoside | 2.0 |
| Polysorbate 80, Glycerol. Gossypium Herbaceum | 1.0 |
| (Cotton) Seed Oil, Water (Aqua) | |
| Trideceth-9, PEG-5 Ethylhexanoate, Water (Aqua) | 1.0 |
| Glycereth-90 Isostearate, Laureth-2 | 0.5 |
| Sodium Laureth Sulfate | 37.0 |
| Glycerol. Triticum Vulgare (Wheat) Gluten. Water (Aqua) | 1.0 |
| Sodium Chloride | 0.3 |
| Perfume oil PO1, PO2, PO3, PO4, or PO5 | 1.0 |
| SymOcide BHO (Hydroxyacetophenone, Benzyl alcohol, | 1.5 |
| Caprylyl glycol, Water) | |
| Avenanthramide L | 0.25 |
| Avenanthramide A | 0.15 |

TABLE 38
Mineral wash and cleaning gel

| Ingredients | INCI | Amount |
|---|---|---|
| Water | Water (Aqua) | ad 100 |
| Pionier ® NP 37 G | Sodium Carbomer | 1.5 |
| SymSol ® PF-3 | Water (Aqua), Pentylene Glycol, | 5.0 |
| | Sodium Lauryl Sulfoacetate, | |
| | Sodium Oleoyl Sarcosinate, | |
| | Sodium Chloride, Disodium | |
| | Sulfoacetate, Sodium Oleate, | |
| | Sodium Sulfate | |
| Hydroviton ® 24 | Water (Aqua), Pentylene Glycol. | 1.0 |
| | Glycerol, Sodium Lactate, Lactic | |
| | Acid, Serine, Urea, Sorbitol, | |
| | Sodium Chloride, Allantoin | |
| Extrapone ® Silk GW | Water (Aqua), Glycerol, | 1.0 |
| | Hydrolyzed Silk | |
| Hydrolite ® 5 | Pentylene Glycol | 4.0 |
| Actipearls Red Star # | Water (Aqua), Propylene Glycol, | 1.0 |
| DH10402/6 | Algin, Gellan Gum, Xanthan | |
| | Gum, CalciumChloride, Cl 12490 | |
| | (Pigment Red 5), Mica (Cl | |
| | 77019), Titanium Dioxide (Cl | |
| | 77891) | |
| Perfume oil PO1, PO2, | Fragrance | 0.5 |
| PO3, PO4, or PO5 | | |
| SymGuard CD | Phenylpropanol, o-cymen-5-ol, | 0.3 |
| | Decylene glycol | |
| SymSave H | Hydroxyacetophenone | 0.5 |
| Avenanthramide L | Avenanthramide L | 0.001 |
| Avenanthramide A | Avenanthramide A | 0.03 |

TABLE 39
After shave tonic

| Ingredients | INCI | Amount |
|---|---|---|
| SymSol ® PF-3 | Water (Aqua), Pentylene | 3.0 |
| | Glycol, Sodium Lauryl | |
| | Sulfoacetate, Sodium Oleoyl | |
| | Sarcosinate, Sodium Chloride, | |
| | Disodium Sulfoacetate, | |
| | SodiumOleate, Sodium | |
| | Sulfate | |
| SymSitive ® 1609 | Pentylene Glycol, 4-t- | 1.0 |
| | Butylcyclohexanol | |
| Frescolat ® ML | Menthyl Lactate | 0.3 |
| Glycerol 99.5 P. | Glycerol | 5.0 |
| Water | Water (Aqua) | ad 100 |
| Extrapone ® Glacier | Glycerol, Water (Aqua) | 1.0 |
| Water GW | | |
| SymCalmin ® | Butylene Glycol, Pentylene | 0.5 |
| | Glycol, Hydroxyphenyl | |
| | Propamidobenzoic Acid | |
| Dragosine ® | Carnosine | 0.1 |
| Hydrolite ® 5 | Pentylene Glycol | 5.0 |
| SymClariol | Decylene Glycol | 0.1 |
| Ethanol 96% | Alcohol Denat. | 5.0 |
| Colour Pigment | Colour Pigment | 0.05 |
| Perfume oil PO1, PO2, | Fragrance | 0.15 |
| PO3, PO4, or PO5 | | |
| SymSave H | Hydroxyacetophenone | 0.8 |
| Avenanthramide L | Avenanthramide L | 0.001 |
| Avenanthramide A | Avenanthramide A | 0.002 |

TABLE 40
Hair conditioner with Crinipan, rinse-off

| Ingredients | INCI | Amount |
|---|---|---|
| Lanette ® O | Cetearyl Alcohol | 4.0 |
| Dragoxat 89 | Ethylhexyl Isononanoate | 2.0 |
| Genamin ® KDM-P | Behentrimonium Chloride | 1.0 |
| SymClariol | Decylene Glycol | 0.2 |
| SF 1550 | Phenyl Trimethicone | 0.1 |
| Neo Heliopan ® BB | Benzophenone-3 | 0.1 |
| Crinipan ® AD | Climbazole | 0.4 |
| Glycerol 99.5 P. | Glycerol | 6.0 |
| Water | Water (Aqua) | ad 100 |
| Actipone ® Alpha Pulp | Water (Aqua), Butylene Glycol, | 0.5 |
| | Malic Acid, Actinidia Chinensis | |
| | (Kiwi) Fruit Juice, Citrus | |
| | Aurantium Dulcis (Orange) | |
| | Juice, Citrus Paradisi | |
| | (Grapefruit) Juice, Pyrus Malus | |
| | (Apple) Juice, Trideceth-9, | |
| | Prunus Amygdalus Dulcis | |
| | (Sweet Almond) Seed Extract | |
| Extrapone ® Bamboo P | Propylene Glycol, Water | 0.5 |
| | (Aqua), Butylene Glycol, | |
| | Bambusa Vulgaris Shoot | |
| | Extract | |
| Sodium Hydroxide 10% | Sodium Hydroxide | 0.4 |
| solution | | |
| Colour I | Colour | 0.6 |
| Colour II | Colour | 0.3 |
| Perfume oil PO1, PO2, | Fragrance | 0.4 |
| PO3, PO4, or PO5 | | |
| Hydrolite-6 | 1,2 Hexanediol | 0.5 |
| Avenanthramide L | Avenanthramide L | 0.0005 |
| SymSave H | Hydroxyacetophenone | 0.3 |
| Avenanthramide A | Avenanthramide A | 0.001 |

TABLE 41
Scalp soothing hair conditioner with
UV-B/UV-A protection, rinse off

| Ingredients | INCI | Amount |
|---|---|---|
| Avenanthramide L | Avenanthramide L | 0.0005 |
| SymSave H | Hydroxyacetophenone | 0.5 |
| Avenanthramide A | Avenanthramide A | 0.0025 |
| Hydrolite-5 Green | Pentylene Glycol | 0.1 |
| Abil 350 | Dimethicone | 0.1 |
| Dehyquart A CA | Cetrimonium Chloride | 0.5 |
| Dehyquart SP | Quaternium-52 | 4.0 |
| Dracorin ® CE | Glyceryl Stearate Citrate | 1.0 |
| EDETA BD | Disodium EDTA | 0.1 |
| Extrapone ® Green Tea | Glycerin, Water (Aqua), | 0.7 |
| GW | Camellia Sinensis Leaf Extract | |
| Perfume oil PO1, PO2, | Fragrance | 0.5 |
| PO3, PO4, or PO5 | | |
| Lara Care A-200 | Galactoarabinan | 0.5 |
| Neutral Oil | Caprylic/Capric Triglyceride | 1.0 |
| PCL Liquid 100 | Cetearyl Ethylhexoate | 0.3 |
| PCL Solid | Stearyl Heptanoate, Stearyl | 3.0 |
| | Caprylate | |
| SymOcide ®PS | Phenoxyethanol, Decylene | 0.5 |
| | Glycol, 1,2-Hexanediol | |
| Water (demineralized) | Water (Aqua) | ad 100 |

TABLE 42
Hair conditioner with UV protection

| Ingredients | INCI | Amount |
|---|---|---|
| Renex PEG 6000 | PEG-150 | 2.5 |
| Hair Conditioner Base | Cetyl alcohol, Behentrimonium | 3.0 |
| | chloride, Triticum Vulgare | |
| | (Wheat) bran extract, Linoleic | |
| | acid | |
| PCL-Solid | Stearyl heptanoate, stearyl | 0.5 |
| | caprylate | |
| Dow Corning 5200 | Laurylmethicone copolyol | 0.5 |
| Natrosol 250 HR | Hydroxyethylcellulose | 0.5 |
| Benzophenone-4 | Benzophenone-4 | 1.0 |
| Neo Heliopan AP | Disodiumphenyldibenz- | 1.0 |
| | imidazole tetrasulphonate | |
| Amino methyl propanol | Amino methyl propanol | 2.0 |
| Dow Corning 949 | Amodimethicone, Cetrimonium | 2.0 |
| cationic emulsion | chloride, Trideceth-12 | |
| Perfume oil PO1, PO2, | Fragrance | 0.8 |
| PO3, PO4, or PO5 | | |
| Hydrolite-8 | Caprylyl Glycol | 0.3 |
| SymSave H | Hydroxyacetophenone | 0.5 |
| Avenanthramide L | Avenanthramide L | 0.002 |
| Avenanthramide A | Avenanthramide A | 0.01 |
| Water | Water (Aqua) | ad 100 |

TABLE 43
Hair conditioner, leave on

| Ingredients | INCI | Amount |
|---|---|---|
| Avenanthramide L | Avenanthramide L | 0.015 |
| SymSave H | Hydroxyacetophenone | 0.3 |
| Hydrolite-5 Green | Pentylene Glycol | 2.5 |
| Dehyquart A CA | Cetrimonium Chloride | 0.2 |
| Dehyquart SP | Quaternium-52 | 2.0 |
| Dracorin ® CE | Glyceryl Stearate Citrate | 1.0 |
| Drago-Calm | Water, Glycerin, Avena Sativa | 2.0 |
| | (Oat) Kernel Extract | |
| Farnesol | Farnesol | |
| Perfume oil PO1, PO2, | Fragrance | 0.5 |
| PO3, PO4, or PO5 | | |
| Lara Care A-200 | Galactoarabinan | 0.1 |
| Polymer JR 400 | Polyquaternium-10 | 0.1 |
| Propylene Glycol | Propylene Glycol | 0.8 |
| SymMollient ® WS | Trideceth-9. PEG-5 | 1.0 |
| | Isononanoate. Water | |
| SymSol ®PF3 | Water, Pentylene Glycol, | 1.5 |
| | Sodium Lauryl Sulfoacetate, | |
| | Sodium Oleoyl Sarcosinate, | |
| | Sodium Chloride, Disodium | |
| | Sulfoacetate, Sodium Oleate, | |
| | Sodium Sulfate | |
| SymTriol ® | Caprylyl Glycol, 1,2- | 0.6 |
| | Hexanediol, Methylbenzyl | |
| | Alcohol | |
| Water (demineralized) | Water (Aqua) | ad 100 |

TABLE 44
Anti-itch hair conditioner, leave on

| Ingredients | INCI | Amount |
|---|---|---|
| Avenanthramide L | Avenanthramide L | 0.5 |
| Hydrolite-5 Green | Pentylene Glycol | 1.0 |
| SymSave H | Hydroxyacetophenone | 1.0 |
| Avenanthramide A | Avenanthramide A | 0.1 |
| (−)-alpha Bisabolol | Bisabolol | 0.1 |
| Dehyquart A CA | Cetrimonium Chloride | 0.5 |
| Dehyquart SP | Quaternium-52 | 4.0 |
| Dracorin ® CE* | Glyceryl Stearate Citrate | 1.0 |
| Drago-Oat-Active* | Water (Aqua), Butylene Glycol, | 2.0 |
| | Avena Sativa (Oat) Kernel | |
| | Extract | |
| Perfume oil PO1, PO2, | Fragrance | 0.1 |
| PO3, PO4, or PO5 | | |
| Lara Care A-200 | Galactoarabinan | 1.5 |
| Neutral Oil | Caprylic/Capric Triglyceride | 1.0 |
| PCL Liquid 100* | Cetearyl Ethylhexoate | 0.3 |
| Polymer JR 400 | Polyquaternium-10 | 0.1 |
| Propylene Glycol | Propylene Glycol | 0.8 |
| SymGlucan ® | Aqua, Glycerin, 1,2-Hexandiol, | 5 |
| | Caprylyl Glycol, Beta-Glucan | |
| SymMollient ® W/S | Trideceth-9, PEG-5 | 2.0 |
| | Isononanoate, Water (Aqua) | |
| SymSol ®PF3 * | Water, Pentylene Glycol, | 1.5 |
| | Sodium Lauryl Sulfoacetate, | |
| | Sodium Oleoyl Sarcosinate, | |
| | Sodium Chloride, Disodium | |
| | Sulfoacetate, Sodium Oleate, | |
| | Sodium Sulfate | |
| Water, demineralized | Water (Aqua) | ad 100 |

TABLE 45
Sprayable hair conditioner with zinc pyrithrione, leave-on

| Ingredients | INCI | Amount |
|---|---|---|
| Monomuls 60-35 C | Hydrogenated Palm Glycerides | 1.7 |
| Cetiol OE | Dicaprylyl Ether | 7.2 |
| Abil 100 | Dimethicone | 3.6 |
| Dehyquart F 75 | Distearoylethyl | 4.0 |
| | Hydroxyethylmonium | |
| | Methosulfate, Cetearyl Alcohol | |
| Eumulgin B1 | Ceteareth-12 | 3.5 |
| Cetiol S | Diethylhexylcyclohe xane | 7.2 |
| D-Panthenol | Panthenol | 0.1 |
| Glycerol 99.5 P. | Glycerol | 1.5 |
| Water | Water (Aqua) | ad 100 |
| Actipone ® Rosemary | Water (Aqua), PropyleneGlycol, | 0.1 |
| | Rosmarinus Officinalis | |
| | (Rosemary) Leaf Extract | |
| Frescolat ® ML Cryst. | Menthyl Lactate | 0.5 |
| Dragosantol100 | Bisabolol | 0.1 |
| Zinc Omadine | Zinc pyrithione | 0.1 |
| Perfume oil PO1, PO2, | Fragrance | 0.4 |
| PO3, PO4, or PO5 | | |
| 2-Phenoxyethyl alcohol | Phenoxyethanol | 0.4 |
| Hydrolite-5 Green | Pentylene Glycol | 3.0 |
| SymDiol 68 | 1,2-Hexanediol, Caprylyl glycol | 0.3 |
| Avenanthramide L | Avenanthramide L | 0.001 |
| Avenanthramide A | Avenanthramide A | 0.001 |

TABLE 46
Hair styling gel

| Ingredients | Amount |
|---|---|
| Water | ad 100 |
| PVM/MA Decadiene Crosspolymer | 0.6 |
| PVP | 3.0 |
| Isocetyl Stearate | 4.0 |
| Ethylhexyl Methoxycinnamate | 0.5 |
| Hydrolite-6 (1,2 Hexanediol) | 0.5 |
| Aminomethyl Propanol | 0.4 |
| Perfume oil PO1, PO2, PO3, PO4, or PO5 | 0.6 |
| SymDiol ® 68T (1,2-Hexanediol, 1,2-Octanediol, | 0.4 |
| Tropolone) | |
| Phenoxyethanol | 0.3 |
| Avenanthramide L | 0.0005 |
| Avenanthramide A | 0.00025 |

TABLE 47
Deodorant stick

| Ingredients | Amount |
|---|---|
| Sodium stearate | 8.0 |
| PPG-3 Myristyl ether | 70.0 |
| 1,2-propylene glycol | 10.0 |
| 1,1-dimethyl-3-phenylpropanol | 0.2 |
| 2-butyloctanoic acid | 0.2 |
| Perfume oil PO1, PO2, PO3, PO4, or PO5 | 0.6 |
| Water | ad 100 |
| SymDeo Plus | 0.5 |
| (Jasmol (2-benzlheptanol), 1-Dodecanol (Lauryl Alcohol), | |
| 1,2-Decanediol (Decylene Glycol), 2-Phenoxyethyl | |
| Alcohol (Phenoxyethanol)) | |
| Avenanthramide L | 0.001 |
| Hydrolite-8 (Caprylyl Glycol) | 0.4 |
| Avenanthramide A | 0.001 |

TABLE 48
Zirconium suspensoid antiperspirant stick

| Ingredients | INCI | Amount |
|---|---|---|
| PCL Liquid 100 | Cetearyl ethylhexanonate | ad 100 |
| Silicone Fluid 345 | Cyclomethicone | 10.0 |
| CRODACOL C90 | Cetyl Alcohol | 8.0 |
| SYNCROWAX HGLC | C18-36 Triglyceride | 8.0 |
| CRODAMOL PTC | Pentaerythritol | 5.0 |
| | Tetracaprylate/Caprate | |
| SymDeo MPP | Dimethyl Phenylbutanol | 0.3 |
| SYNCROWAX HRC | Tribehenin | 4.0 |
| VOLPO N5 | Oleth-5 | 1.0 |
| Titanium Dioxide | | 1.0 |
| Rezal 36GP | Aluminium Tetrachlorohydrex | 20.0 |
| | GLY | |
| Dry Flo C | Aluminium Starch Octenyl | 22.5 |
| | Succinate | |
| Symlite G8 | Glyceryl caprylate | 0.3 |
| Perfume oil PO1, PO2, | Fragrance | 0.6 |
| PO3, PO4, or PO5 | | |
| Avenanthramide L | Avenanthramide L | 0.0005 |
| Hydrolite-5 Green | Pentylene Glycol | 5.0 |
| Avenanthramide A | Avenanthramide A | 0.002 |

TABLE 49
Antiperspirant/deodorant roll-on

| Ingredients | INCI | Amount |
|---|---|---|
| Avenanthramide L | Avenanthramide L | 0.001 |
| Hydrolite-6 | 1,2 Hexanediol | 0.8 |
| Avenanthramide B | Avenanthramide A | 0.004 |
| Dragosantol ® 100* | Bisabolol | 0.1 |
| Ethanol 96% | Ethanol | 30.0 |
| Farnesol | Farnesol | 0.5 |
| Perfume oil PO1, PO2, | Fragrance | 1.5 |
| PO3, PO4, or PO5 | | |
| Frescolat ®ML cryst, | Menthyl Lactate | 0.2 |
| Irgasan DP 300 | Triclosan | 0.3 |
| Natrosol 250 HHR | Hydroxyethyl-cellulose | 0.3 |
| Solubilizer 611674 | PEG-40 Hydrogenated Castor | 2.0 |
| | Oil, Trideceth-9, Water (Aqua) | |
| SymDeo ® B125 | 2-Methyl 5-Cyclohexylpentanol | 0.5 |
| Water (demineralized) | Water (Aqua) | ad 100 |
| Zirkonal L 450 | Aluminium Zirconium | 37.0 |
| | Pentachloro-hydrate | |
| | (40% aqueous solution) | |

TABLE 50
Deodorant formulation in the form of a roll-on gel

| Ingredients | Amount |
|---|---|
| 1,3-butylene glycol | 2.0 |
| 2-Methyl 5-cyclohexylpentanol | 0.1 |
| PEG-40-hydrogenated castor oil | 2.0 |
| Hydroxyethylcellulose | 0.5 |
| Pentylene Glycol (Hydrolite-5 Green) | 1.0 |
| Perfume oil PO1, PO2, PO3, PO4, or PO5 | 0.3 |
| 1,3-propanediol | 0.5 |
| SymGuard CD (3-Phenylpropanol, o-cymen-3-ol, | 0.4 |
| Decylene glycol) | |
| Ethylhexyl glycerin | 0.1 |
| SymSave H (Hydroxyacetophenone) | 0.5 |
| Avenanthramide L | 0.001 |
| Avenanthramide B | 0.004 |
| Water | ad 100 |

TABLE 51
Clear deo anti-perspirant roll-on

| Ingredients | INCI | Amount |
|---|---|---|
| Methocel E4M Premium | Hydroxypropyl Methylcellulose | 0.5 |
| Water | Water (Aqua) | ad 100 |
| Neo-PCL Water Soluble | Trideceth-9, PEG-5 | 1.0 |
| N | Ethylhexanoate, Water (Aqua) | |
| Solubilizer | PEG-40 Hydrogenated Castor | 3.0 |
| | Oil, Trideceth-9, Propylene | |
| | Glycol, Water (Aqua) | |
| Deolite | Dimethyl Phenylpropanol, | 0.5 |
| | Pentylene Glycol | |
| Locron LW | Aluminium Chlorohydrate | 25.0 |
| Aloe Vera Gel | Aloe Barbadensis Leaf Juice | 1.0 |
| Concentrate 10/1 | | |
| 1,2-Propylene Glycol 99 | Propylene Glycol | 4.0 |
| P GC | | |
| Ethanol 96% | Alcohol Denat. | 30.0 |
| Perfume oil PO1, PO2, | Fragrance | 1.0 |
| PO3, PO4, or PO5 | | |
| SymDiol 68 | 1,2 Hexabediol, Caprylyl Glycol | 1.0 |
| Avenanthramide A | Avenanthramide A | 0.005 |
| Avenanthramide L | Avenanthramide L | 0.001 |
| Avenanthramide A | Avenanthramide A | 0.005 |

TABLE 52
Deodorant pump spray with SymClariol

| Ingredients | INCI | Amount |
|---|---|---|
| SymClariol ® | Decylene Glycol | 0.2 |
| Solubilizer | PEG-40 Hydrogenated Castor | 4.0 |
| | Oil, Trideceth-9, Propylene | |
| | Glycol, Water (Aqua) | |
| Neo-PCL Water Soluble | Trideceth-9, PEG-5 | 1.5 |
| N | Ethylhexanoate, Aqua | |
| SymRelief ® | Bisabolol, Zingiber Officinale | 0.1 |
| | (Ginger) Root Extract | |
| Water | Water (Aqua) | ad 100 |
| 1,2-Propylene Glycol | Propylene Glycol | 6.0 |
| Perfume oil PO1, PO2, | Perfume | 0.4 |
| PO3, PO4, or PO5 | | |
| SymDiol ® 68 | 1,2-Hexanediol, Caprylyl Glycol | 0.5 |
| Avenanthramide A | Avenanthramide A | 0.15 |
| Avenanthramide L | Avenanthramide L | 0.2 |
| Avenanthramide A | Avenanthramide A | 0.1 |

TABLE 53
Whitening deodorant spray

| Ingredients | Amount |
|---|---|
| PEG-40-hydrogenated castor oil (Eumulgin HRE 40) | 3.0 |
| Ethylhexylglycerol (Octoxyglycerol) | 0.2 |
| Symbright 2036 (Sclareolide) | 0.1 |
| Ethanol | 40.0 |
| Citrate buffer | 0.5 |
| 1,2-Hexanediol, 1,2-Octanediol (SymDiol 68) | 0.8 |
| SymOcide C (o-cymen-5-ol) | 0.05 |
| 2-Benzylheptan-1-ol (Jasmol) | 0.1 |
| Perfume oil PO1, PO2, PO3, PO4, or PO5 | 0.75 |
| Phenoxyethanol | 0.4 |
| Avenanthramide L | 0.001 |
| SymSave H (Hydroxyacetophenone) | 0.5 |
| Water | ad 100 |

TABLE 54
Deodorant Aoerosol Spray

| Ingredients | Amount |
|---|---|
| Perfume oil PO1, PO2, PO3, PO4, or PO5 | 0.75 |
| Decylene Glycol (SymClariol) | 0.2 |
| Avenanthramide A | 0.002 |
| SymSave H (Hydroxyacetophenone) | 0.2 |
| Disiloxane | ad 100 |
| Isoadipate | 5.0 |
| C12-C15 Alkyl Benzoate | 10.0 |
| Tocopheryl Acetate | 0.5 |
| Farnesol | 0.3 |

40% bulk, charged with 60% Propane/Butane

TABLE 55
Sunscreen lotion (o/w, broadband protection)

| Ingredients | INCI | Amount |
|---|---|---|
| Avenanthramide A | Avenanthramide A | 0.005 |
| Hydrolitre-5 Green | Pentylene Glycol | 3.0 |
| Avenanthramide B | Avenanthramide B | 0.005 |
| Carbopol Ultrez-10 | Carbomer | 0.2 |
| Dow Corning 246 Fluid | Cyclohexasiloxane and | 2.0 |
| | Cyclopentasiloxane | |
| Dragosantol ® 100* | Bisabolol | 0.3 |
| EDETA BD | Disodium EDTA | 0.1 |
| Emulsiphos ® | Potassium Cetyl Phosphate, | 1.5 |
| | Hydrogenated Palm Glycerides | |
| Perfume oil PO1, PO2, | Fragrance | 0.4 |
| PO3, PO4, or PO5 | | |
| Frescolat ®MGA | Menthone Glycerol Acetal | 0.3 |
| Glycerol 85% | Glycerin | 4.7 |
| Keltrol RD | Xanthan Gum | 0.2 |
| Lanette O | Cetearyl Alcohol | 1.0 |
| Neo Heliopan ® 357 | Butyl Methoxy-dibenzoyl- | 1.0 |
| | methane | |
| Neo Heliopan ® AP | Disodium Phenyl | 10.0 |
| (10% as sodium salt) | Dibenzimidazole Tetrasulfonate | |
| Neo Heliopan ® AV | Ethylhexyl Methoxy-cinnamate | 3.0 |
| Neo Heliopan ® Hydro | Phenylbenz-imidazole Sulfonic | 6.7 |
| (15% as sodium salt) | Acid | |
| Neo Heliopan ® MBC | 4-Methylbenzyl-idene Camphor | 1.5 |
| Neo Heliopan ® OS | Ethylhexyl Salicylate | 5.0 |
| Neutral Oil | Caprylic/Capric Triglyceride | 2.0 |
| SymMatrix ® | Maltodextrin, Rubus Fruticosus | 0.3 |
| | (Blackberry) Leaf Extract | |
| SymOcide ® BHO | Hydroxyacetophenone, Benzyl | 1.5 |
| | alcohol, Caprylyl glycol, Aqua | |
| Tegosoft TN | C12-15 Alkyl Benzoate | 5.0 |
| Tocopherol Acetate | Tocopheryl Acetate | 0.5 |
| Triethanolamine, 99% | Triethanolamine | 0.5 |
| Water (demineralized) | Water (Aqua) | ad 100 |

TABLE 56
Emulsion with UV-A/B-broadband protection

| Ingredients | INCI | Amount |
|---|---|---|
| Avenanthramide A | Avenanthramide A | 0.005 |
| SymSave H | Hydroxyacetophenone | 0.5 |
| Avenanthramide B | Avenanthramide B | 0.005 |
| Abil 350 | Dimethicone | 0.3 |
| Butylene Glycol | Butylene Glycol | 3.0 |
| Carbopol Ultrez-10 | Carbomer | 0.2 |
| Citric Acid 10% sol. | Citric Acid | 0.3 |
| Dragosantol ® 100* | Bisabolol | 0.1 |
| EDETA BD | Disodium EDTA | 0.1 |
| Emulsiphos ® | Potassium Cetyl Phosphate, | 1.5 |
| | Hydrogenated Palm Glycerides | |
| Perfume oil PO1, PO2, | Fragrance | 0.1 |
| PO3, PO4, or PO5 | | |
| Frescolat ®X-COOL | Menthyl Ethylamido Oxalate | 1.0 |
| Glyceryl Stearate | Glyceryl Stearate | 2.0 |
| Keltrol RD | Xanthan Gum | 0.2 |
| Lanette 16 | Cetyl Alcohol | 1.2 |
| Lanette E | Sodium Cetearyl Sulfate | 0.7 |
| Neo Heliopan ® AP (10% | Disodium Phenyl | 22.0 |
| as sodium salt) | Dibenzimidazole Tetrasulfonate | |
| Neo Heliopan ® HMS | Homosalate | 5.0 |
| Neutral Oil | Caprylic/Capric Triglyceride | 2.0 |
| PCL Liquid 100 | Cetearyl Ethylhexoate | 3.0 |
| Sodium Hydroxide | Sodium Hydroxide | 2.8 |
| (10% sol.) | | |
| Symdiol ®68 | 1,2-Hexanediol, Caprylylglycol | 0.5 |
| SymMollient ®S | Cetearyl Nonanoate | 1.5 |
| SymSitive ® 1609 | Pentylene Glycol, 4-t- | 0.5 |
| | Butylcyclohexanol | |
| SymWhite ®377 | Phenylethyl Resorcinol | 0.5 |
| Tocopherol Acetate | Tocopheryl Acetate | 0.5 |
| Water (demineralized) | Water (Aqua) | ad 100 |

TABLE 57
Sun protection soft cream (w/o; SPF 40)

| Ingredients | INCI | Amount |
|---|---|---|
| Dehymuls PGPH | Polyglyceryl-2 | 5.0 |
| | dipolyhydroxystearate | |
| Copherol 1250 | Tocopheryl acetate | 0.5 |
| Permulgin 3220 | Ozocerite | 0.5 |
| Zinc stearate | Zinc stearate | 0.5 |
| Tegosoft TN | C12-15 Alkyl benzoate | 10.0 |
| Neo Heliopan ® E1000 | Isoamyl-p-methoxycinnamate | 2.0 |
| Neo Heliopan ® 303 | Octocrylene | 5.0 |
| Neo Heliopan ® MBC | 4-Methylbenzylidene camphor | 3.0 |
| Zinc oxide, neutral | Zinc oxide | 5.0 |
| Water, distilled | Water (aqua) | ad 100 |
| EDETA BD | Disodium EDTA | 0.1 |
| Glycerol | Glycerol | 4.0 |
| Magnesium sulfate | Magnesium sulfate | 0.5 |
| Perfume oil PO1, PO2, | Fragrance | 0.3 |
| PO3, PO4, or PO5 | | |
| Symdiol ® 68 | 1,2-Hexanediol, Caprylylglycol | 0.7 |
| Avenanthramide L | Avenanthramide L | 0.001 |
| Avenanthramide A | Avenanthramide A | 0.005 |
| SymSave H | Hydroxyacetophenone | 0.5 |

TABLE 58
Sun protection milk (w/o)

| Ingredients | INCI | Amount |
|---|---|---|
| Dehymuls PGPH | Polyglyceryl-2 | 3.0 |
| | dipolyhydroxystearate | |
| Beeswax 8100 | Beeswax | 1.0 |
| Monomuls 90-0-18 | Glyceryl oleate | 1.0 |
| Zinc stearate | Zinc stearate | 1.0 |
| Hydrolite-8 | Caprylyl Glycol | 0.3 |
| Cetiol SN | Cetearyl isononanoate | 5.0 |
| Cetiol OE | Dicaprylyl ether | 5.0 |
| Tegosoft TN | C12-15 alkyl benzoate | 4.0 |
| Vitamin E | Tocopherol | 0.5 |
| Neo Heliopan ® OS | Ethylhexyl salicylate | 5.0 |
| Neo Heliopan ® AV | Ethylhexyl methoxycinnamate | 7.5 |
| Uvinul ® T150 | Ethylhexyl triazone | 1.5 |
| Water. distilled | Water (Aqua) | ad 100 |
| Trilon BD | Disodium EDTA | 0.1 |
| Glycerol | Glycerol | 5.0 |
| Neo Heliopan ® AP | Disodium phenyl | 15.0 |
| 10% solution. | dibenzimidazole tetrasulfonate | |
| neutralized with NaOH | | |
| Perfume oil PO1, | Fragrance | 0.25 |
| PO2, PO3, PO4, or | | |
| PO5 | | |
| Alpha bisabolol | Bisabolol | 0.1 |
| SymOcide ® PT | Phenoxyethanol. Tropolone | 0.25 |
| SymSave H | Hydroxyacetophenone | 0.8 |
| Avenanthramide A | Avenanthramide A | 0.002 |
| Avenanthramide B | Avenanthramide B | 0.002 |

TABLE 59
Sun spray with UV-A/B-broadband protection with low oil content

| Ingredients | INCI | Amount |
|---|---|---|
| Avenanthramide L | Avenanthramide L | 0.001 |
| Hydrolite-6 | 1,2 Hexanediol | 0.5 |
| SymSave H | Hydroxyacetophenone | 0.5 |
| Avenanthramide A | Avenanthramide A | 0.002 |
| | | 0.05 |
| Ethanol 96% | Ethanol | 13.0 |
| Perfume oil PO1, PO2, | Fragrance | 0.5 |
| PO3, PO4, or PO5 | | |
| Glyceryl Stearate | Glyceryl Stearate | 4.0 |
| Hydroviton ® PLUS | Water, Pentylene Glycol, | 1.0 |
| | Glycerin, Fructose, Urea, Citric | |
| | Acid, Sodium Hydroxide, | |
| | Maltose, Sodium PCA, Sodium | |
| | Chloride, Sodium Lactate, | |
| | Trehalose, Allantoin, Sodium | |
| | hyaluronate, Glucose | |
| Isoadipate ® | Diisopropyl Adipate | 1.0 |
| Neo Heliopan ® AV | Ethylhexyl Methoxy-cinnamate | 25.0 |
| Neo Heliopan ® MBC | 4-Methylbenzyl-idene Camphor | 33.3 |
| Propylene Glycol | Propylene Glycol | 0.8 |
| Tego Betain L7 | Cocamidopropyl Betaine | 1.0 |
| Water (demineralized) | Water (Aqua) | ad 100 |

TABLE 60
Sunscreen spray (o/w; SPF 15-20)

| Ingredients | INCI | Amount |
|---|---|---|
| Dracorin ® GOC | Glyceryl Oleate Citrate, | 2.0 |
| | Caprylic/Capric Triglyceride | |
| Corapan ® TQ | Diethylhexyl 2,6-Naphthalate | 3.0 |
| Neo Heliopan ® HMS | Homosalate | 7.0 |
| Neo Heliopan ® OS | Ethylhexyl Salicylate | 5.0 |
| Neo Heliopan ® 357 | Butyl Methoxydibenzoylmethane | 3.0 |
| Isoadipate | Diisopropyl Adipate | 6.0 |
| Baysilone ® Oil M10 | Dimethicone | 1.0 |
| Edeta ® BD | Disodium EDTA | 0.1 |
| Vitamin E Acetate | Tocopheryl Acetate | 0.5 |
| Dragosantol ® 100 | Bisabolol | 0.1 |
| Pemulen ® TR-2 | Acrylates/C10-30 Alkyl Acrylate | 0.25 |
| | Crosspolymer | |
| Water | Water (Aqua) | ad 100 |
| Glycerol 99.5 P | Glycerol | 4.0 |
| Butylene Glycol | Butylene Glycol | 5.0 |
| Neo Heliopan ® Hydro | Phenylbenzimidazole Sulfonic | 8.0 |
| (103089), used as 25% | Acid | |
| aq, solution neutralized | | |
| with Biotive ® L-Arginine | | |
| Biotive ® L-Arginine | Arginine | 0.55 |
| Perfume oil PO1, PO2, | Fragrance | 0.4 |
| PO3, PO4, or PO5 | | |
| SymOcide PS | Phenoxyethanol, 1,2-Hexanediol, | 1.5 |
| | Decylene glycol | |
| Avenanthramide B | Avenanthramide B | 0.005 |
| Avenanthramide L | Avenanthramide L | 0.05 |
| Avenanthramide A | Avenanthramide A | 0.05 |

TABLE 61
After sun gel

| Ingredients | INCI | Amount |
|---|---|---|
| SymSol ® PF-3 | Water (Aqua), Pentylene Glycol, | 3.0 |
| | Sodium Lauryl Sulfoacetate, | |
| | Sodium Oleoyl Sarcosinate, | |
| | Sodium Chloride, Disodium | |
| | Sulfoacetate, Sodium Oleate, | |
| | Sodium Sulfate | |
| Glycerol 99.5 P | Glycerol | 5.0 |
| SymHelios ® 1031 | Benzylidene | 0.1 |
| | Dimethoxydimethylin danone | |
| Water | Water (Aqua) | ad 100 |
| Pemulen ® TR-2 | Acrylates/C10-30 Alkyl Acrylate | 1.0 |
| | Crosspolymer | |
| D-Panthenol 75 W | Panthenol | 0.5 |
| SymFinity ® 1298 | Echinacea Purpurea Extract | 0.1 |
| Extrapone ® Pearl GW | Water (Aqua), Glycerol, | 1.0 |
| | Hydrolyzed Pearl, Xanthan Gum | |
| Sodium Hydroxide | Sodium Hydroxide | 2.5 |
| 10% solution | | |
| Ethanol 96% | Alcohol Denat. | 15.0 |
| Perfume oil PO1, PO2, | Fragrance | 0.2 |
| PO3, PO4, or PO5 | | |
| SymOcide ® PS | Phenoxyethanol, 1,2- | 0.8 |
| | Hexanediol, Decyleneglycol | |
| Hydrolite-5 Green | Pentylene Glycol | 0.5 |
| Avenanthramide A | Avenanthramide A | 0.05 |

TABLE 62
After sun lotion

| Ingredients | Amount |
|---|---|
| Acrylate/C10-30 alkylacrylate crosspolymer | 0.4 |
| Cetearylethyl hexanoate | 15.0 |
| Bisabolol | 0.2 |
| Tocopheryl acetate | 1.0 |
| Panthenol | 1.0 |
| Alcohol | 15.0 |
| Glycerol | 3.0 |
| Perfume oil PO1, PO2, PO3, PO4, or PO5 | 0.30 |
| 1,2-Hexanediol (Hydrolite-6) | 1.0 |
| Triethanolamine | 0.2 |
| Pentylene glycol (Hydrolite-5 Green) | 4.0 |
| Aqua dem. | ad 100 |
| 4-Hydroxyacetophenone (SymSave H) | 0.3 |
| Avenanthramide A | 0.005 |

TABLE 63
Syndet antimicrobial soap bar

| Ingredients | INCI | Amount |
|---|---|---|
| Zetesap 813 A | Disodium Lauryl | ad 100 |
| | Sulfosuccinate, Sodium Lauryl | |
| | Sulfate, Corn Starch, Cetearyl | |
| | Alcohol, Paraffin, Titanium | |
| | Dioxide | |
| Amphotensid GB 2009 | Disodium | 6.0 |
| | Cocoamphodiacetate | |
| Allantoin | Allantoin | 1.0 |
| Perfume oil PO1, PO2, | Fragrance | 1.0 |
| PO3, PO4, or PO5 | | |
| SymOcide C | o-cymen-5-ol | 0.1 |
| Symdiol 68 | 1,2 Hexanediol, Caprylyl | 0.5 |
| | Glycol | |
| Avenanthramide L | Avenanthramide L | 0.001 |
| Avenanthramide B | Avenanthramide B | 0.005 |
| Avenanthramide A | Avenanthramide A | 0.005 |

TABLE 64
Syndet soap bar

| Ingredients | INCI | Amount |
|---|---|---|
| Fenopon AC-78 | Sodium Cocoyl Isethionate | 20.0 |
| Natriumlaurylsulfoacetate | Sodium Lauryl Sulfoacetate | 16.0 |
| Paraffin | Paraffin | 19.0 |
| Wax. microcrystalline | Microcrystalline Wax | 1.0 |
| Corn Starch | Corn Starch | 8.0 |
| Coconut acid | Coconut acid | 2.0 |
| Lauric acid diethanol | Lauramide DEA | 2.0 |
| amide | | |
| Dextrin | Dextrin | 21.0 |
| Lactic acid, 88% | Lactic Acid | 1.0 |
| SymGuard CD | 3-Phenylpropanol, o-cymen- | 0.3 |
| | 5-ol, Decylene glycol | |
| Thymol | Thymol | 0.05 |
| Symlite G8 | Glyceryl Caprylate | 0.2 |
| Water | Water | ad 100 |
| Perfume oil PO1, PO2, | Fragrance | 1.0 |
| PO3, PO4, or PO5 | | |
| Avenanthramide L | Avenanthramide L | 0.001 |
| Hydrolite-6 O | 1,2 Hexanediol | 0.5 |
| Avenanthramide A | Avenanthramide A | 0.003 |

TABLE 65
Shaving foam

| Ingredients | Amount |
|---|---|
| Dem. Water | ad 100 |
| Triethanolamine | 4.0 |
| Edenor L2 SM (Stearinic acid, Palmitinic acid) (Cognis) | 5.3 |
| Laureth-23 | 3.0 |
| Stearylalcohol | 0.5 |
| Avenanthramide L | 0.001 |
| Hydrolite-5 (Pentylene Glycol) | 2.5 |
| Avenanthramide B | 0.003 |
| Sodium lauryl sulfate | 3.0 |
| Extrapone Seaweed (Water, Propylene glycol, | 1.0 |
| Potassium iodide, Fucus Vesiculosus Extract) | |
| Dragosantol (Bisabolol, Farnesol) | 0.1 |
| Perfume oil PO1, PO2, PO3, PO4, or PO5 | 1.0 |
| Propane, butane 4,2 Bar | 4.0 |

TABLE 66
Sprayable disinfecting gel

| Ingredients | INCI | Amount |
|---|---|---|
| Water | Water (Aqua) | ad 100 |
| Stabileze QM | PVM/Ma Decadiene | 0.25 |
| | Crosspolymer | |
| Sodium Hydroxide 10% | Sodium Hydroxide | 0.4 |
| solution | | |
| Coffein pure | Caffeine | 0.5 |
| Extrapone ® Horse | Propylene Glycol, Water (Aqua), | 1.0 |
| Chestnut | Glucose, Aesculus | |
| | Hippocastanum (Horse | |
| | Chestnut) Seed Extract, Lactic | |
| | Acid | |
| Hydrolite ® 5 | Pentylene Glycol | 3.0 |
| 1,3 Butylene Glycol | Butylene Glycol | 5.0 |
| Biotive ® Esculin | Esculin | 0.3 |
| Sesquihydrate | | |
| Ethanol 96% | Alcohol Denat. | 10.0 |
| Solubilizer | PEG-40 Hydrogenated Castor | 0.5 |
| | Oil, Trideceth-9, Water (Aqua) | |
| Perfume oil PO1, PO2, | Fragrance | 0.2 |
| PO3, PO4, or PO5 | | |
| Octenidine | Octenidine dihydrochloride | 0.1 |
| dihydrochloride | | |
| Phenoxyethanol | Phenoxyethanol | 0.5 |
| SymSave H | Hydroxyacetophenone | 0.4 |
| Avenanthramide L | Avenanthramide L | 0.0005 |
| Avenanthramide A | Avenanthramide A | 0.003 |
| Avenanthramide B | Avenanthramide B | 0.002 |

TABLE 67
Solution for wet wipes

| Ingredients | INCI | Amount |
|---|---|---|
| SymSol ® PF-3 | Water (Aqua), Pentylene Glycol, | 2.0 |
| | Sodium Lauryl Sulfoacetate, | |
| | SodiumOleoyl Sarcosinate, | |
| | Sodium Chloride, Disodium | |
| | Sulfoacetate, SodiumOleate, | |
| | Sodium Sulfate | |
| Dragosantol ® 100 | Bisabolol | 0.1 |
| Glycerol 99.5 P | Glycerol | 5.0 |
| Water | Water (Aqua) | ad 100 |
| Hydrolite ® 5 | Pentylene Glycol | 5.0 |
| D-Panthenol 75 W | Panthenol | 0.8 |
| DragoCalm ® | Water (Aqua), Glycerol, Avena | 1.0 |
| | Sativa (Oat) Kernel Extract | |
| Witch Hazel-Distillate | Hamamelis Virginiana (Witch | 1.0 |
| | Hazel) Water, Water (Aqua), | |
| | Alcohol | |
| Allplant Essence ® | Pelargonium Graveolens | 1.0 |
| Org, Rose Geranium P | Flower/Leaf/Stem Water | |
| Perfume oil PO1, PO2, | Fragrance | 0.1 |
| PO3, PO4, or PO5 | | |
| Avenanthramide L | Avenanthramide L | 0.2 |
| SymSave H | Hydroxyacetophenone | 0.6 |
| Avenanthramide B | Avenanthramide A | 0.3 |

TABLE 68
Further preferred cleansing formulations without
sodium lauryl ether sulfate (SLES) (% (w/w)).

| INCI | 1 | 2 | 3 | 4 | 5 | 6 | 7 | 8 | 9 | 10 |
|---|---|---|---|---|---|---|---|---|---|---|
| Avenanthramide A, B, | 1.0 | — | 0.5 | — | — | 1.5 | 1.0 | — | — | 2.0 |
| C and L ≥ 100 ppm | | | | | | | | | | |
| (oat extract fraction) in | | | | | | | | | | |
| sum in glycerine/water | | | | | | | | | | |
| Hydroxyacetophenone | 0.5 | 0.6 | 0.3 | 0.8 | 0.2 | 0.5 | 0.5 | 0.5 | 0.3 | 0.1 |
| (SymSave H) | | | | | | | | | | |
| 1,2 Hexanediol, | 0.5 | 0.5 | 1.0 | 0.3 | 1.0 | 0.5 | 1.0 | 0.5 | 0.5 | 0.2 |
| Caprylyl Glycol | | | | | | | | | | |
| (Symdiol 68) | | | | | | | | | | |
| 1,2 Hexanediol, | — | — | 0.7 | — | — | — | 0.7 | — | — | — |
| Caprylyl Glycol, | | | | | | | | | | |
| Tropolone (Symdiol 68 | | | | | | | | | | |
| T) | | | | | | | | | | |
| Ammonium Lauryl | — | 5.0 | — | — | — | — | — | — | — | — |
| Sulfate | | | | | | | | | | |
| (Stepanol AM) | | | | | | | | | | |
| Aqua, Glycerin, | — | — | — | — | — | 0.3 | — | — | — | — |
| Echinacea Purpurea | | | | | | | | | | |
| Extract (Extrapone | | | | | | | | | | |
| Echinacea) | | | | | | | | | | |
| Aqua, Pentylene | — | — | — | — | — | 3.0 | — | — | — | — |
| Glycol, Sodium Lauryl | | | | | | | | | | |
| Sulfoacetate, Sodium | | | | | | | | | | |
| Oleoyl Sarcosinate, | | | | | | | | | | |
| Sodium Chloride, | | | | | | | | | | |
| Sodium Oleate | | | | | | | | | | |
| (SymSol PF3) | | | | | | | | | | |
| Bisabolol (Dragosantol | — | — | — | — | 0.1 | — | — | — | — | — |
| 100) | | | | | | | | | | |
| Butyrospermum Parkii | | | | | | | | | | 13.0 |
| Butter | | | | | | | | | | |
| (Cetiol SB 45) | | | | | | | | | | |
| Caprylic/Capric | — | — | — | — | — | — | — | — | — | 2.0 |
| Triglyceride | | | | | | | | | | |
| Hydroxymethoxyphenyl | | | | | | | | | | |
| Decanone | | | | | | | | | | |
| (Symdecanox HA) | | | | | | | | | | |
| Caprylyl Glycol, 1,2 | — | — | — | — | 0.8 | — | — | — | 0.8 | — |
| Hexanediol, | | | | | | | | | | |
| Methylbenzyl Alcohol | | | | | | | | | | |
| Citric Acid 30% | — | — | — | 3.0 | — | — | — | | — | — |
| aqueous sol. | | | | | | | | | | |
| Climbazole (Crinipan | — | — | — | — | — | — | — | 0.5 | — | — |
| AD) | | | | | | | | | | |
| Cocamide MEA | — | — | — | — | — | — | — | 3.0 | — | — |
| (Mackamide CMA) | | | | | | | | | | |
| Cocamide MIPA | 0.5 | — | — | — | — | — | — | — | — | — |
| Cocoamidopropyl | 15.0 | 5.0 | 3.0 | 6.0 | 14.0 | — | — | 15.0 | 17.0 | — |
| Betaine | | | | | | | | | | |
| (Tego Betain F50) | | | | | | | | | | |
| Coco Betaine (Dehyton | — | — | — | — | — | — | 2.0 | — | — | — |
| AB 30) | | | | | | | | | | |
| Coco-Glucoside | — | 10.0 | — | — | — | — | — | — | — | — |
| (Plantacare 818 UP) | | | | | | | | | | |
| Dedcylene Glycol | 0.1 | — | — | 0.05 | — | 0.2 | — | — | 0.1 | — |
| Decyl Glycoside | — | — | — | — | 2.0 | — | — | — | — | — |
| (Ecosense 3000) | | | | | | | | | | |
| Disodium Cocoyl | — | 3.0 | — | — | — | — | — | — | — | — |
| Glutamate (Plantapon | | | | | | | | | | |
| ACG LC) | | | | | | | | | | |
| Disodium EDTA (EDTA | — | — | — | — | — | — | — | 0.1 | — | — |
| BD) | | | | | | | | | | |
| Disodium Lauryl | — | — | 2.0 | 2.0 | — | — | — | — | — | — |
| Sulfosuccinate | | | | | | | | | | |
| (Setacin F spezial) | | | | | | | | | | |
| Fragrance PO1, PO2, | 1.0 | 0.5 | 0.1 | 0.3 | 0.1 | 0.3 | 0.5 | 0.3 | 0.05 | — |
| PO3, PO4, or PO5 | | | | | | | | | | |
| Glycerin 99% | — | — | 0.5 | — | 3.0 | — | — | — | — | — |
| Glycerin, Aqua, | — | — | — | — | 1.0 | — | — | — | — | — |
| Hamamelis Virginia | | | | | | | | | | |
| Bark/Leaf/Twig Extract | | | | | | | | | | |
| (Extrapone Witch | | | | | | | | | | |
| Hazel GW) | | | | | | | | | | |
| Glyceryl Caprylate | 0.1 | 0.5 | 0.2 | 0.3 | 0.5 | 0.1 | — | — | — | — |
| (Symlite G8) | | | | | | | | | | |
| Glycol Distearate, | — | — | — | 2.0 | — | — | — | 3.0 | — | — |
| Laureth-4, | | | | | | | | | | |
| Cocoamidopropyl | | | | | | | | | | |
| Betaine (Quickpearl | | | | | | | | | | |
| PK3) | | | | | | | | | | |
| Isostearamide MlPA, | — | — | — | 1.0 | — | — | — | | — | — |
| Glyceryl Laurate (Antil | | | | | | | | | | |
| SPA 80) | | | | | | | | | | |
| Kaolin | — | — | — | — | — | — | — | — | — | 18.8 |
| (ImerCare ® 02K-S) | | | | | | | | | | |
| Lactic Acid 90% | — | — | — | — | 0.3 | — | — | | — | — |
| aqueous sol. | | | | | | | | | | |
| Lauroyl/Myristoyl | 12.0 | — | — | — | — | — | — | | — | — |
| Methyl Glucamide | | | | | | | | | | |
| (Glucotain Clean) | | | | | | | | | | |
| Lauryl Hydroxysultaine | — | — | — | — | — | — | 11.0 | | — | — |
| (45% AS) | | | | | | | | | | |
| Lauryl Lactate | — | — | — | — | — | — | 0.3 | | — | — |
| (Schercemol LL Ester) | | | | | | | | | | |
| Maltodextrin, | — | — | — | — | — | — | — | — | — | 0.5 |
| Lactobacillus Ferment | | | | | | | | | | |
| (SymReboot L19) | | | | | | | | | | |
| Menthyl Lactate | — | — | — | — | — | — | — | 0.2 | — | — |
| (Frescolat ML) | | | | | | | | | | |
| PEG-200 | — | — | — | 4.5 | — | — | — | — | — | — |
| Hydrogenated Glyceryl | | | | | | | | | | |
| Palmate, PEG-7 | | | | | | | | | | |
| Glyceryl Cocoate | | | | | | | | | | |
| (Antil 200) | | | | | | | | | | |
| PEG-4 | — | — | — | — | — | — | 2.0 | — | 2.8 | — |
| Rapeseedamide (92% | | | | | | | | | | |
| AS) | | | | | | | | | | |
| PEG-40 Hydrogenated | — | — | — | — | 1.5 | — | — | 0.9 | — | — |
| Castor Oil, Trideceth-9, | | | | | | | | | | |
| Propylene Glycol, | | | | | | | | | | |
| Aqua (Solubilizer | | | | | | | | | | |
| Symrise) | | | | | | | | | | |
| PEG-45 M | — | — | — | — | — | — | — | 0.15 | — | — |
| (PolyoxWSR N 60K) | | | | | | | | | | |
| Pentylene Glycol | 0.5 | 1.0 | — | — | — | 2.0 | — | — | — | 1.5 |
| (Hydrolite-5 Green) | | | | | | | | | | |
| Pentylene Glycol, 4-t- | — | — | — | — | — | 1.0 | — | — | — | — |
| Butylcyclohexanol | | | | | | | | | | |
| (Symsitive 1609) | | | | | | | | | | |
| Pentylene Glycol, | — | — | — | — | 1.0 | — | — | 2.0 | — | — |
| Butylene Glycol, | | | | | | | | | | |
| Hydroxyphenyl | | | | | | | | | | |
| Propamidobenzoic | | | | | | | | | | |
| Acid (SymCalmin) | | | | | | | | | | |
| Phenyl Propanol, O- | — | — | — | — | 0.5 | — | — | — | — | — |
| Cymen-5-ol, Decylene | | | | | | | | | | |
| Glycol (Symguard CD) | | | | | | | | | | |
| Piroctone Olamine | 0.1 | — | 0.5 | — | — | — | — | — | — | — |
| (Octopirox) | | | | | | | | | | |
| Polyacrylate 33 | — | — | — | — | — | — | — | 6.5 | — | — |
| (Rheomer 33T) | | | | | | | | | | |
| Polyquaternium-10 | — | — | — | 0.3 | — | — | — | 0.2 | — | — |
| (Polymer JR 400) | | | | | | | | | | |
| Polyquaternium-7 | 0.4 | — | — | — | — | — | — | — | — | — |
| (Dehyquart CC7) | | | | | | | | | | |
| Polysilicone -19 (Abil | — | — | — | 2.0 | — | — | — | — | — | — |
| UV Quat 50) | | | | | | | | | | |
| Potassium Sorbate | — | — | 0.3 | 0.4 | — | 0.5 | — | — | — | 0.3 |
| Propylene Glycol | — | — | — | — | 3.0 | — | — | — | — | — |
| Rhamnose | — | — | — | — | — | 0.5 | — | — | — | — |
| Sodium C14-C16 | — | — | — | — | — | — | 27.0 | — | — | — |
| Olefin Sulfonate (38% | | | | | | | | | | |
| AS) | | | | | | | | | | |
| Sodium Chloride | 0.5 | — | — | | — | — | — | — | — | — |
| Sodium | — | — | — | 6.0 | — | — | — | — | — | — |
| Cocoamphoacetate | | | | | | | | | | |
| (Rewoteric AMC) | | | | | | | | | | |
| Sodium Cocoyl | — | — | — | — | — | 2.0 | — | — | — | — |
| Alaninate | | | | | | | | | | |
| (Amilite ACS 12) | | | | | | | | | | |
| Sodium Cocoyl | — | 5.0 | 3.0 | — | — | — | — | — | — | — |
| Glutamate (Hostapon | | | | | | | | | | |
| CCG) | | | | | | | | | | |
| Sodium Cocoyl | 10.0 | — | — | — | — | — | — | — | — | — |
| Glycinate (Hostapon | | | | | | | | | | |
| SG) | | | | | | | | | | |
| Sodium Cocoyl | 4.5 | — | — | — | — | — | — | — | — | 15.0 |
| Isethionate (ELFAN ® | | | | | | | | | | |
| AT 84) | | | | | | | | | | |
| Sodium Hydroxide | — | — | — | — | — | — | — | 0.5 | — | — |
| (50% aqueous sol.) | | | | | | | | | | |
| Sodium Laureth-5 | — | — | — | — | — | — | — | — | 8.0 | — |
| Carboxylate | | | | | | | | | | |
| (Akypo Foam RL 40) | | | | | | | | | | |
| Sodium Laureth-6 | — | — | — | — | — | 1.0 | — | — | — | — |
| Carboxylate | | | | | | | | | | |
| (Akypo SOFT 45 HP) | | | | | | | | | | |
| Sodium Lauroyl | 3.0 | — | — | — | — | — | — | — | — | — |
| Glutamate (Hostapon | | | | | | | | | | |
| CLG) | | | | | | | | | | |
| Sodium Lauroyl | — | — | — | — | 2.0 | — | — | — | — | — |
| Lactylate (Dermosoft | | | | | | | | | | |
| SLL) | | | | | | | | | | |
| Sodium Lauroyl Methyl | — | — | — | 22.0 | — | — | — | — | — | — |
| Isethionate (Iselux LQ- | | | | | | | | | | |
| CLR SB) | | | | | | | | | | |
| Sodium Lauroyl | — | 3.0 | — | 3.0 | — | — | — | — | — | — |
| Sarcosinate (Protelan | | | | | | | | | | |
| LS 9011) | | | | | | | | | | |
| Sodium Lauryl Glucose | | | | | | | | 6.0 | | |
| Carboxylate | | | | | | | | | | |
| Lauryl Glucoside | | | | | | | | | | |
| (Plantapon LGC Sorb) | | | | | | | | | | |
| Sodium Myristoyl | — | — | — | — | — | — | — | — | — | 30.0 |
| Glutamate | | | | | | | | | | |
| (Amisoft MS11) | | | | | | | | | | |
| Sodium Salicylate | — | — | 0.3 | — | — | — | — | — | — | — |
| (Seboclear) | | | | | | | | | | |
| Sorbitol | 1.0 | — | — | — | — | — | — | — | — | — |
| Trideceth-9, PEG-5 | — | — | — | — | — | — | — | 2.0 | — | — |
| Isononanoate, Water | | | | | | | | | | |
| (Aqua) | | | | | | | | | | |
| Water (Aqua), | — | — | 1.0 | — | | — | — | — | — | — |
| Glycerin, Tetraselmis | | | | | | | | | | |
| Suecica Extract | | | | | | | | | | |
| (SymControl Care) | | | | | | | | | | |
| Xanthan Gum (Keltrol | — | 0.5 | — | — | 0.5 | — | — | — | — | — |
| RD) | | | | | | | | | | |

| | |
|---|---|
| Water (Aqua) | Ad 100 |

1: Mild hair and body wash
2: Shampoo
3: Anti acne face wash
4: Color care shampoo
5: Feminine sash
6: MicellarwWater
7: Liquid soap
8: Antidandruff shampoo
9: Baby shampoo
10: Solid shampoo.

1. A composition comprising:
at least one avenanthramide, selected from the group of avenanthramides A, B, C, D, E, F, G, H, L and mixtures of these avenanthramides; and
at least one penetration enhancer.

2. The composition according to claim 1, wherein the at least one avenanthramide is obtained from an oat source of the oat species Avena sativa or Avena nuda.

3. The composition according to claim 1, wherein the penetration enhancer is selected from the group of diols, straight-chain 1,2-alkanediols having from 3 to 12 carbon atoms, polyols, alcohols, dimethyl isosorbide (INCI), triethyl citrate, butylene carbonate, glycerine carbonate, dipropylene glycol, and a mixture of any of the foregoing.

4. The composition according to claim 1, comprising:
0.0001 to 5.0 wt % of the at least one avenanthramide, and
0.01 to 10.0 wt % of the at least one penetration enhancer,
based on the total weight of the composition.

5. A method of using the composition according to claim 1 for skin protection, skin care, scalp protection, scalp care, hair care, nail care or the prevention and/or treatment of skin conditions, intolerant or sensitive skin irritation, skin reddening, wheals, pruritus (itching), skin aging, wrinkle formation, loss of skin volume, loss of skin elasticity, pigment spots, pigment abnormalities, or dry skin, or aging, the method comprising applying the composition of claim 1 to a subject in need thereof.

6. A method of using the composition according to claim 1 as a medicament, the method comprising administering the composition to a subject in need thereof.

7. The method according to claim 6, wherein the composition is administered for the prevention and/or treatment of dermatological or keratological diseases, and/or for the prevention and/or treatment of dermatological diseases associated with increased ROS production.

8. The method of claim 7, wherein the dermatological or keratological diseases are selected from the group of atopic dermatitis (neurodermitis), psoriasis, acneiform exanthema, sebostasis, xerosis, eczema, hyper seborrhea and hypo seborrhea, dermatitis, rosacea, erythema, pruritus, inflammation, irritation, fibrosis, Lichen planus, Pityriasis rosea, Pityriasis versicolor, autoimmune bullous diseases, urticaria, angioedema, allergic skin reactions, wound healing, and tissue regeneration.

9. A method for preparing a cosmetic or pharmaceutical preparation comprising the step of including the composition of claim 1 in a cosmetic or pharmaceutical preparation.

10. A cosmetic or pharmaceutical preparation comprising the composition according to claim 1.

11. The cosmetic or pharmaceutical preparation according to claim 10, further comprising one or more active substances selected from the group of skin-moisturising and/or moisture-retaining substances, cooling agents, osmolytes, keratological substances, nurturing substances, anti-inflammatory, antibacterial or antimycotic substances, substances having a reddening-alleviating or itch-alleviating action, lenitive substances, mixtures of the foregoing, cosmetically or pharmaceutically acceptable excipients selected from the group of antioxidants, preservatives, chelating agents, surface-active substances, emulsifiers, perfume oils, anti-foaming agents, colorants, pigments having a colouring action, thickeners, plasticisers, fats, oils, waxes, alcohols, polyols, polymers, foam stabilisers, electrolytes, organic solvents silicone derivatives, and a mixture of any of the foregoing.

12. The cosmetic or pharmaceutical preparation according to claim 10 in the form of a fluid, tincture, lotion, gel, cream, ointment, spray or shampoo.

13. A method for preparing a composition according to claim 1, comprising the steps of:
providing at least one avenanthramide; and
adding and mixing in at least one penetration enhancer to the at least one avenanthramide.

14. The composition of claim 3, wherein the penetration enhancer is selected from one or more of 1,2-propanediol (propylene glycol), 1,2-butanediol, 1,2-pentanediol (Hydrolite-5), 1,2-hexanediol (Hydrolite-6), 1,2-heptanediol, 1,2-octanediol, 1,2-nonanediol, 1,2-decanediol or 1,2-dodecane diol, 1,3-butanediol (butylene glycol), 1,3-propanediol, 1,4-butanediol, and 1,1′-oxydi-2-propanol (dipropylene glycol) and its isomers.

15. The composition of claim 4, wherein the at least one avenanthramide is present in an amount of 0.001 to 1.0 wt %, based on the total weight of the composition.

16. The composition of claim 4, wherein the at least one penetration enhancer is present in an amount of 0.1 to 5.0 wt %, based on the total weight of the composition.

17. The cosmetic or pharmaceutical preparation of claim 10, wherein the composition is present in an amount of 0.0001 to 10% by weight, based on the total weight of the cosmetic or pharmaceutical preparation.
